(19) United States
(12) Patent Application Publication
(10) Pub. No.: US 2022/0177510 A1
(43) Pub. Date: Jun. 9, 2022

(54) DERIVATIVES OF GYLCERO-MANNO-HEPTOSE ADP FOR USE IN MODULATING IMMUNE RESPONSE

(71) Applicant: Shanghai Yao Yuan Biotechnology Co., Ltd., Shanghai (CN)

(72) Inventors: Tian Xu, Shanghai (CN); Cong Xu, Shanghai (CN); Danyang Liu, Shanghai (CN); Jieqing Fan, Shanghai (CN); Yanfang Pan, Shanghai (CN)

(21) Appl. No.: 17/594,634

(22) PCT Filed: Apr. 24, 2020
(86) PCT No.: PCT/CN2020/086691

§ 371 Date: Oct. 25, 2021

(30) Foreign Application Priority Data
Apr. 26, 2019 (CN) PCT/CN2019/084585

(51) Int. Cl. C07H 19/207; A61K 39/39; A61K 35/17; A61K 38/17; A61K 39/00; A61K 38/19; A61K 38/20; A61K 39/395; A61K 31/7076; A61P 37/04; A61P 31/20

(52) U.S. Cl. CPC C07H 19/207; A61K 2039/55561; A61K 35/17; A61K 38/1774; A61K 39/00117; A61K 39/001186; A61K 39/001192; A61K 38/193; A61K 38/2046; A61K 38/208; A61K 38/2086; A61K 38/20; A61K 39/3955; A61K 31/7076; A61P 37/04; A61P 31/20; A61K 39/39

(57) ABSTRACT

The disclosure provides compounds, compositions and methods related to activating alpha-kinase 1 (ALPK1) for modulating an immune response and treating or preventing cancer, infection, inflammation and related diseases and disorders as well as potentiating an immune response to a target antigen. The disclosure also provides heterocyclic compounds of formula (I) as agonists of alpha protein kinase 1 (ALPK1) and their use in activating ALPK1, modulating an immune response and treating diseases such as cancer, wherein A¹, A², L¹, L², L³, Z¹, Z², W¹, W², R¹, R², R³, R⁴, R⁵, R⁶ and R⁷ are as defined herein.

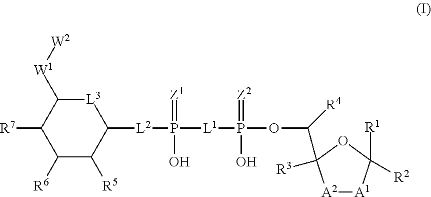

### FIELD OF THE INVENTION

The present invention relates to compounds that are derivatives of certain bacterial metabolites in the ADP-heptose biosynthetic pathway, compositions comprising same, and methods for their use in therapy.

### BACKGROUND OF THE INVENTION

The studies on mechanism of inflammatory response have identified various protein kinases that act as essential signaling components. Defects in protein kinase are frequently associated with the pathogenesis of human inflammatory diseases, cancer and diabetes.

Alpha-kinases are a unique protein kinase superfamily, displaying little sequence similarity to typical protein kinases. A total of six alpha kinase members including alpha-protein kinase 1 (ALPK1), ALPK2, ALPK3, elongated factor-2 kinase (eEF2K), and transient receptor potential cation channel M6 and M7 (TRPM6 and TRPM7) have been identified (Ryazanov A G et al., Curr Biol 1999 9(2):R43-45; Ryazanov A G et al., Proc Natl Acad Sci USA 1997 94(10):4884-4889). ALPK1 was initially identified as a new component of raft-containing sucrose-isomerase (SI) vesicles in epithelial cells (Heinet M et al., J. Biol. Chem. 2005 280(27): 25637-43). It was shown that ALPK1 phosphorylates myosin 1 and plays an essential role in the exocytic transport to the apical plasma membrane. A transposon-inserted homozygous inactivating mutation of ALPK1 in mice resulted in motor coordination deficits which could be rescued by overexpressing full-length ALPK1 (Chen M et al., BMC Neurosci. 2011 12:1).

Genetic association studies implicated ALPK1 in risk for gout, chronic kidney disease, myocardial infarction, and diabetes (Wang S J et al., J. Mol. Med. 2011 89:1241-51; Ko A M et al., J. Intl. Epidemiol. 2013 42: 466-474; Chiba T et al., Human Cell 2015 28:1-4; Yamada Y et al. J Med Genet 2013 50:410-418; Fujimaki T et al., Biomed Report 2014 2:127-131; Shimotaka S et al., Biomed Report 1 2013 940-44; Yamada Y et al., Biomed. Report 2015 DOI: 10.3892/br.2015.439).

ALPK1 activation has also been implicated in cancer, including lung, colorectal, and breast cancers (Liao et al. Scientific Reports 2016 6:27350; Strietz et al., Oncotarget 2016 1-16).

Recent studies have implicated ALPK1 as an important regulator of the innate immune response activated by certain bacteria. For example, APLK1 was suggested to be a key regulator of innate immunity against bacteria through its promotion of TIFA oligomerization and interleukin 8 (IL-8) expression in response to infection with S. flexneri, S. typhimurium, and Neisseria meningitides (Milivojevic et al., PLoS Pathog 2017 13(2): e1006224). Zimmerman et al. describe an ALPK1 and TIFA dependent innate immune response triggered by the Helicobacter pylori Type IV Secretion System. (Zimmermann et al., Cell Reports 2017 20(10): 2384-95). Both of these studies suggest that the bacterial metabolite, heptose-1,7-bisphosphate (HBP) activates TIFA-dependent innate immunity.

There are many diseases, disorders, and conditions whose clinical manifestations result from inflammation and various infections. There is a need for new methods for modulating inflammation in target tissues for treating such diseases, disorders, and conditions. The present disclosure addresses this need by providing compounds that are derivatives of certain metabolites downstream from HBP in the ADP-heptose biosynthetic pathway.

### SUMMARY OF THE INVENTION

The present invention is based, in part, on the discovery that certain derivatives of bacterial metabolites, and specifically derivatives of D-glycero-D-manno-heptose-1β-ADP (H1BADP) have surprising biological activity. H1BADP is downstream of D-glycero-β-D-manno-heptose 1,7-bisphosphate (heptose 1,7 bisphosphate or “HBP”) in the E. coli biosynthetic pathway shown in FIG. 1. The present inventors previously characterized this metabolite as well as D-glycero-β-D-manno-heptose-1-phosphate (HMP1BP) and L-glycero-D-manno-heptose-1β-ADP (H1B-ADP-6L) as able to activate ALPK1 dependent proinflammatory signal transduction. That biological activity was unexpected in view of the current knowledge of ALPK1 and its role in activation of innate immunity by bacterial metabolites. The E. coli H1b-ADP biosynthetic pathway is shown in FIG. 1.

The present disclosure provides additional derivatives of H1BADP represented by formulas I, IA, IB, IC, 1D, and 1E described herein, having improved chemical and/or biological properties compared to the parent molecules.

Accordingly, the present disclosure provides compounds, compositions comprising same, including pharmaceutical compositions, and methods related to modulating an immune response, treating cancer, potentiating an immune response to a target antigen, treating a liver disease or disorder including non-alcoholic steatohepatitis (NASH) and diseases and disorders caused by the hepatitis C virus (HCV) and the hepatitis B virus (HBV), and treating or preventing a disease or disorder caused by an infectious agent as described herein through administration of a compound represented by formula I, including formulas I, IA, IB, IC, 1D, or 1E described herein. In some embodiments, the disclosure provides methods of modulating an immune response in a subject, the methods comprising administering to the subject a composition comprising a compound represented by formula I, IA, IB, IC, 1D, or 1E described herein.

The present disclosure provides compounds represented by formula (I) compound represented by Formula I, or a stereoisomer, a stable isotope, prodrug or pharmaceutically acceptable salt thereof:

The present invention discloses novel heterocyclic compounds as agonists of ALPK1. The compounds are represented by formula (I):

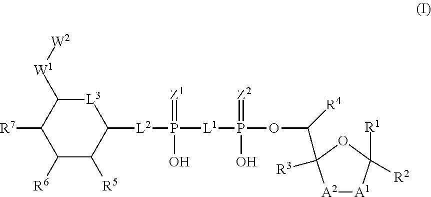

wherein A¹, A², L¹, L², L³, Z¹, Z², W¹, W², R¹, R², R³, R⁴, R⁵, R⁶ and R⁷ are as defined herein. Also included within the scope of the disclosure are stereoisomers, tautomers, stable isotopes, prodrugs, and pharmaceutically acceptable salts of the compounds of Formula I.

A¹ and A² are independently selected from O, S and —C(R⁸R⁹)—, wherein R⁸ and R⁹ are independently selected from H, D, —OH, N₃, —CN, halogen and an optional substituted group selected from C1-C4 alkyl, C1-C4 alkoxy, C1-C4 haloalkyl, C1-C4 haloalkoxy, C1-C4 alkanoyloxy, C1-C4 alkanoyloxy and aralkyloxy, wherein the optional substituents are 1-3 substituents independently selected from D, halogen, —OH, ═O, C1-C4 alkyl and C1-C4 alkoxy; at least one of A¹ or A² is —C(R⁸R⁹); wherein R⁸ or R⁹ in A¹ can cyclize with R⁸ or R⁹ in A² to form C3-C6 cycloalkyl and heterocyclyl containing 3 to 9 ring members and having 1-3 heteroatoms selected from N, O and S as ring members, each optionally substituted by 1-3 substituents independently selected from D, halogen, —OH, ═O, C1-C4 alkyl and C1-C4 alkoxy;

L¹ and L² are independently selected from O, CH₂, CHF and CF₂;

L³ is O, S, CH₂ or CH(OH);

Z¹ and Z² are independently selected from O and S;

W¹ is —C(R¹⁰R¹¹)—, wherein R¹⁰ and R¹¹ are independently selected from H, D, —OH, halogen, and optionally substituted groups selected from C1-C4 alkyl, C1-C4 alkoxy, C1-C4 haloalkyl, C1-C4-haloalkoxy, C1-C4 alkenyloxy, aralkyloxy, and 1-6 membered oligopeptidyl linked via C-terminal C(O)O— and R¹²CO₂—, wherein R¹² is selected from C1-C20 alkyl, C1-C20 alkenyl, C1-C20 alkoxy, C1-C20 alkenyloxy, C1-C20 alkylamino, C3-C6 cycloalkyl, heterocyclyl containing 4 to 6 ring members and having 1-3 heteroatoms selected from N, O and S as ring members, aryl, and heteroaryl containing 5 to 10 ring atoms and having 1-3 heteroatoms selected from N, O and S as ring members and 1-6 membered oligopeptidyl linked via N-terminal N; wherein the optional substituents for R¹⁰ and R¹¹ are 1-3 substituents independently selected from D, halogen, —OH, ═O, C1-C4 alkyl and C1-C4 alkoxy;

When W¹ is —C(R¹⁰R¹¹)—, wherein R¹⁰ is F, and the others are defined as above, W² is H or C1-C3 alkyl optionally substituted with 1-3 substituents independently selected from D, halogen, —OH, ═O, C1-C3 alkoxyl, C1-C3 haloalkyl, C1-C3 haloalkoxyl, C1-C3 alkenyloxyl and R¹²CO₂—, wherein R¹² is C1-C4 alkyl, C1-C4 alkoxy, C1-C4 alkylamino, C3-C6 cycloalkyl, cycloheteroalkyl containing 3 to 6 ring members and having 1-3 heteroatoms selected from N, O and S as ring members, C6-C10 aryl, and heteroaryl containing 5 to 10 ring atoms and having 1-3 heteroatoms selected from N, O and S as ring members;

W² is R¹³-Q¹-W³—, wherein Q¹ is selected from —O— or —NH—; W³ is selected from a bond or C1-C3 alkylene groups optionally substituted with 1-3 substituents independently selected from halogen, —OH, C1-C3 alkoxy, C1-C3 haloalkyl, C1-C3 haloalkoxyl, C1-C3 alkenyloxy; wherein R¹³ is 1-6 membered oligopeptidyl linked via C-terminal carbonyl group or R¹⁴Q²C(O)—; wherein Q² is a bond, —O— or —NH—; R¹⁴ is 1-6 membered oligopeptidyl linked via N-terminal N or an optionally substituted group selected from C5-C20 alkyl, C5-C20 alkylenyl and R¹⁵-Q³-Q⁴-Q⁵-; wherein Q³,Q⁴ and Q⁵ are independently selected from a bond, aryl, heteroaryl containing 5 to 6 ring atoms, C3-C6 cycloalkyl and heterocyclyl containing 4 to 6 ring members and having 1-3 heteroatoms selected from N, O and S as ring members, and at least one of Q³, Q⁴ and Q⁵ is not a bond; R¹⁵ is an optionally substituted group selected from C1-C18 alky and C1-C18 alkoxy, wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy; R¹ is aryl or heteroaryl containing 6 to 10 ring atoms and having 1-4 heteroatoms selected from N, O and S as ring members, wherein R¹ is optionally substituted with 1-3 substituents selected from D, halogen, —OH, ═O, CN, NH₂ and an optionally substituted group selected from C1-C4 alkyl, C1-C4 alkoxy, (R¹⁶R¹⁷)N— and (R¹⁶R¹⁷)NCO—, wherein R¹⁶ and R¹⁷ are independently selected from H and an optionally substituted group selected from C1-C4 alkyl, C3-C6 cycloalkyl, heterocyclyl containing 4 to 6 ring members and having 1-3 heteroatoms selected from N, O and S as ring members, aryl, arylalkyl, heteroaryl containing 5 to 10 ring atoms and having 1-3 heteroatoms selected from N, O and S as ring members; and heteroarylalkyl containing 5 to 10 ring atoms and having 1-3 heteroatoms selected from N, O and S as ring members; wherein the optionally substituents are 1-3 substituents independently selected from from D, halogen, —OH, ═O, CN, NH₂ and an C1-C4 alkyl, C1-C4 alkoxy, C1-C3 haloalkyl, C1-C3 haloalkoxy and C3-C6 cycloalkyl;

R², R³ and R⁴ are independently selected from H, D, halogen, C1-C4 alkyl and C1-C4 haloalkyl;

R⁵, R⁶ and R⁷ are independently selected from H, —OH, halogen, and R¹²CO₂—, and at least two of R⁵, R⁶ and R⁷ are —OH or R¹²CO₂—, wherein R¹² is selected from C1-C4 alkyl, C1-C4 alkoxy, C1-C4 alkanoyloxy, C1-C4 alkenyloxy, C1-C4 alkylamino, C3-C6 cycloalkyl, heterocyclyl containing 4 to 6 ring members and having 1-3 heteroatoms selected from N, O and S as ring members, C6-C10 aryl, and heteroaryl containing 5 to 10 ring atoms and having 1-3 heteroatoms selected from N, O and S as ring members; wherein any two of the adjacent groups of R⁵, R⁶ and R⁷ can cyclize to form heterocyclyl containing 5 to 9 ring members and having 1-3 heteroatoms selected from N, O and S as ring members, each optionally substituted by 1-3 substituents independently selected from D, halogen, —OH, ═O, C1-C4 alkyl and C1-C4 alkoxy.

In embodiments, the disclosure provides a pharmaceutical composition comprising a compound of formula 1 as described herein, and a pharmaceutically acceptable carrier.

In embodiments, the disclosure provides a method for modulating an immune response in a subject in need of such treatment, the method comprising administering to the subject a composition comprising a compound of formula I, IA, IB, IC, 1D, or 1E described herein, and prodrugs, analogs and derivatives thereof. In embodiments, the method for modulating an immune response is selected from activation of innate immunity and activation of adaptive immunity.

In embodiments, the disclosure provides a method for treating cancer in a subject in need of such treatment, the method comprising administering to the subject a composition comprising a compound of formula I, IA, IB, IC, 1D, or 1E described herein, and prodrugs, analogs and derivatives thereof. In embodiments, the cancer is selected from soft tissue sarcoma, breast cancer, head and neck cancer, melanoma, cervical cancer, bladder cancer, hematologic malignancy, glioblastoma, pancreatic cancer, prostate cancer, colon cancer, breast cancer, renal cancer, lung cancer, merkel cell carcinoma, small intestine cancer, thyroid cancer, acute myelogenous leukemia (AML), acute lymphocytic leukemia (ALL), chronic lymphocytic leukemia (CLL), chronic myelogenous leukemia (CML), gastric cancer, gastrointestinal stromal tumors, non-Hodgkins lymphoma, Hodgkins lymphoma, liver cancer, leukemia, lymphoma, T-cell lymphoma, brain cancer, and multiple myeloma. In embodiments, the cancer is selected from breast cancer, head and neck cancer, melanoma, renal cancer, lung cancer, merkel cell carcinoma, and lymphoma.

In embodiments, the disclosure provides a method for potentiating an immune response to a target antigen in a subject, the method comprising administering to the subject a composition comprising a compound of formula I, IA, IB, IC, 1D, or 1E described herein, and prodrugs, analogs and derivatives thereof, as a vaccine or immunologic adjuvant that acts to potentiate an immune response to the target antigen. In embodiments, the target antigen is an antigen of an infectious agent selected from the group consisting of adenovirus, Coxsackie B virus, cytomegalovirus, eastern equine encephalitis virus, ebola virus, enterovirus 71, Epstein-Barr virus, Haemophilus influenzae type b (Hib), hepatitis C virus (HCV), herpes virus, human immunodeficiency virus (HIV), human papillomavirus (HPV), hookworm, Marburg virus, norovirus, respiratory syncytial virus (RSV), rotavirus, Salmonella typhi, Staphylococcus aureus, Streptococcus pyogenes, varicella, West Nile virus, Yersinia pestis, and Zika virus. In embodiments, a compound of formula 1 as described herein, acts as a vaccine adjuvant for a vaccine in the treatment or prevention of anthrax, caries, Chagas disease, dengue, diphtheria, ehrlichiosis, hepatitis A or B, herpes, seasonal influenza, Japanese encephalitis, leprosy, lyme disease, malaria, measles, mumps, meningococcal disease, including meningitis and septicemia, Onchocerciasis river blindness, pertussis (whooping cough), pneumococcal disease, polio, rabies, rubella, schistosomiasis, severe acute respiratory syndrome (SARS), shingles, smallpox, syphilis, tetanus, tuberculosis, tularemia, tick-borne encephalitis virus, typhoid fever, trypanosomiasis, yellow fever, or visceral leishmaniasis.

In embodiments, the disclosure provides a method for treating a disease or disorder amendable to treatment by activation of NFkB, p38, and JNK cell signaling pathways in cells of a subject, the method comprising administering to the subject a composition comprising a compound of formula I, IA, IB, IC, 1D, or 1E described herein, and prodrugs, analogs and derivatives thereof. In embodiments, the disease or disorder is selected from tuberculosis, meningitis, pneumonia, ulcer, sepsis, rhinitis, asthma, allergy, COPD, inflammatory bowel disease, arthritis, obesity, radiation-induced inflammation, psoriasis, atopic dermatitis, non-alcoholic steatohepatitis (NASH), Alzheimer's disease, systemic lupus, erythematosus (SLE), autoimmune thyroiditis (Grave's disease), multiple sclerosis, ankylosing spondylitis bullous diseases, and diseases and disorders caused by the hepatitis C virus (HCV), the hepatitis B virus (HBV), or the human immunodeficiency virus (HIV).

In embodiments, the disclosure provides a method for treating or preventing a disease or disorder caused by an infectious agent selected from a bacteria, virus, or parasite in a subject in need thereof, the methods comprising administering to the subject a composition comprising a compound of formula I, IA, IB, IC, 1D, or 1E described herein, and prodrugs, analogs and derivatives thereof. In embodiments, the infectious agent is a bacteria. In embodiments, the infectious agent is a virus. In embodiments, the infectious agent is a parasite. In embodiments, the bacteria is a Gram-negative or a Gram-positive bacteria. In embodiments, the Gram-negative bacteria is selected from the group consisting of Acinetobacter baumanii, Aggregatobacter actinomycetemcomitans, Bartonella bacilliformis, Bartonella henselae, Bartonella quintana, Bifidobacterium, Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trachomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B typhus, Salmonella, dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus and Yersinia pestitis. In embodiments, the Gram-positive bacteria selected from the group consisting of Actinomycetes, Bacillus anthracis, Bacillus subtilis, Clostridium tetani, Clostridium perfingens, Clostridium botulinum, Clostridium tetani. Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin-resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, and Streptococcus mutants. In embodiments, the virus is selected from the group consisting of ebolavirus, hepatitis B virus, hepatitis C virus, herpes simplex virus, human immunodeficiency virus (HIV), human papillomavirus (HPV-6, HPV-11), human SARS coronavirus, influenza A virus, influenza B virus, influenza C virus, measles virus, rabies virus, poliovirus, SARS corona virus, and yellow fever virus. In embodiments, the parasite is selected from the group consisting of Acanthamoeba spp, American trypanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, Dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

In embodiments of any of the foregoing methods, the method may further comprise administering to the subject one or more additional therapeutic agents or immune modulators, and combinations thereof. In embodiments, the one or more additional therapeutic agents is selected from an anti-microbial agent, such as an anti-bacterial agent, an anti-viral agent, or an anti-parasitic agent, an anti-cancer agent, or a therapeutic agent for the treatment of tuberculosis, meningitis, pneumonia, ulcer, sepsis, rhinitis, asthma, allergy, COPD, inflammatory bowel disease, arthritis, obesity, radiation-induced inflammation, psoriasis, atopic dermatitis, non-alcoholic steatohepatitis (NASH), Alzheimer's disease, systemic lupus, erythematosus (SLE), autoimmune thyroiditis (Grave's disease), multiple sclerosis, and ankylosing spondylitis bullous diseases.

In embodiments of the methods for treating cancer, the one or more additional therapeutic agents is an immune modulator. In embodiments, the immune modulator is selected from one or more of an inhibitor or antagonist of an immune checkpoint regulator, an immune stimulatory molecule, and an agonist of an immune co-stimulatory molecule. In embodiments, the inhibitor or antagonist of an immune checkpoint regulator is a PD-1/PD-L1 inhibitor. In embodiments, the PD-1/PD-L1 inhibitor is selected from the group consisting of nivolumab, pembrolizumab, pidilizumab, BMS-936559, atezolizumab, durvalumab, and avelumab. In embodiments, the immune modulator is selected from interferon alpha (INFα), a stimulator of interferon genes (“STING”) agonist, a TLR agonist (e.g., resquimod), and an anti-OX40 (CD134) agonist antibody. In embodiments, the agonist of an immune co-stimulatory molecule is an anti-OX40 (CD134) agonist antibody. In embodiments, the cancer is selected from advanced melanoma, non-small cell lung cancer, renal cell carcinoma, bladder cancer, Hodgkin's lymphoma, liver cancer, gastric cancer, colon cancer, breast cancer, non-Hodgkin's lymphoma, prostate cancer, head and neck cancer, thyroid cancer, brain cancer, acute myeloid leukemia (AML), merkel cell carcinoma, multiple myeloma, cervical cancer, and sarcoma.

In embodiments, the one or more additional immune modulators is an inhibitor or antagonist of an immune checkpoint regulator, or a vaccine against an immune checkpoint regulator. In embodiments, the one or more additional immune modulators is an agonist of an immune an immune checkpoint regulator, such as a co-stimulatory molecule, for example an agonist of OX40 (CD134). In embodiments, the immune checkpoint regulator is selected from the programed cell death 1 (PD-1) receptor (CD279), a ligand of PD-1 (e.g., PD-L1), cytotoxic T-lymphocyte associated protein 4 (CTLA4), tumor necrosis factor receptor superfamily member 9 (alternatively TNFRSF9, 4-1BB) and 4-1BB ligands, tumor necrosis factor receptor superfamily member 4 (alternatively TNFRSF4, OX40) and OX40 ligands, glucocorticoid-induced TNFR-related protein (GITR), Tumor Necrosis Factor Receptor Superfamily Member 7 (alternatively TNFRSF7, cluster of differentiation 27, CD27), TNFRSF25 and TNF-like ligand 1A (TL1A), TNF Receptor Superfamily Member 5 (alternatively TNFRSF5, CD40) and CD40 ligand, Herpesvirus entry mediator (HVEM)-tumor necrosis factor ligand superfamily member 14 (alternatively TNFSF14, LIGHT)-lymphotoxin alpha (LTA), herpesvirus entry mediator-(HVEM)-B- and T-lymphocyte attenuator (BTLA)-CD160 (alternatively TNFSF14), lymphocyte activating gene 3 (LAG3), T-cell immunoglobulin and mucin-domain containing-3 (TIM3), sialic-acid-binding immunoglobulin-like lectins (SIGLECs), inducible T-cell costimulator (ICOS) and ICOS ligand, B7-H3 (B7 family, alternatively CD276), V-set domain-containing T-cell activation inhibitor 1 (VTCN1, alternatively B7-H4), V-Type immunoglobulin domain-containing suppressor of T-cell activation (VISTA), human endogenous retrovirus-H long terminal repeat-associating protein 2 (HHLA2)-transmembrane and Immunoglobulin domain containing 2 (TMIGD2), butyrophilins, natural killer cell receptor 2B4 (alternatively NKR2B4, CD244) and B-Cell Membrane Protein (CD48), T-Cell Immunoreceptor with Immunoglobulin (Ig) and immunoreceptor tyrosine-based inhibition motif domains (TIGIT) and Poliovirus receptor (PVR) family members, killer-cell immunoglobulin-like receptors (KIRs), Immunoglobulin-like transcripts (ILTs) and leukocyte immunoglobulin-like receptor (LIRs), natural killer group protein 2 member D (NKG2D) and natural killer group protein 2 member A (NKG2A), major histocompatibility complex (MHC) class I polypeptide-related sequence A (MICA) and MHC class I polypeptide-related sequence B (MICB), natural killer cell receptor 2B4 (CD244), colony stimulating factor 1 receptor (CSF1R), indoleamine 2,3-dioxygenase (IDO), transforming growth factor beta (TGFβ), Adenosine-ecto-nucleotidase triphosphate diphosphohydrolase 1 (CD39)-5′-nucleotidase (CD73), C-X-C motif chemokine receptor 4 (CXCR4) and C-X-C motif chemokine ligand 12 (CXCL12), phosphatidylserine, signal regulatory protein alpha (SIRPA) and integrin associated protein (CD47), vascular endothelial growth factor (VEGF), and neuropilin.

In embodiments, the one or more additional immune modulators is a vaccine.

In embodiments of a method for treating cancer, the vaccine is a vaccine against a tumor antigen. In embodiments, the tumor antigen is selected from glycoprotein 100 (gp100), mucin 1 (MUC1), and melanoma-associated antigen 3 (MAGEA3).

In embodiments, the one or more additional immune modulators is a T cell, preferably a chimeric antigen receptor T cell. In embodiments, the one or more additional immune modulators is a recombinant protein, preferably selected from granulocyte-macrophage colony-stimulating factor (GM-CSF), interleukin 7 (IL-7), IL-12, IL-15, IL-18, and IL-21.

In embodiments of any of the foregoing methods, the composition may comprise a compound of formula I, IA, IB, IC, 1D, or 1E described herein, and prodrugs, analogs and derivatives thereof.

In embodiments, the disclosure provides a method for treating a liver disease or disorder in a subject in need of such treatment, the method comprising administering to the subject a compound of formula I, IA, IB, IC, 1D, or 1E described herein, and prodrugs, analogs and derivatives thereof. In embodiments, the liver disease or disorder is selected from liver cancer, non-alcoholic steatohepatitis (NASH), and a disease or disorder caused by infection with the hepatitis C virus (HCV) or the hepatitis B virus (HBV).

In embodiments of any of the foregoing methods, the subject may be a vertebrate. In embodiments, the subject is a human.

The disclosure also provides a vaccine composition or vaccine adjuvant composition comprising a compound of formula I, IA, IB, IC, 1D, or 1E described herein, and prodrugs, analogs and derivatives thereof, and a carrier.

In embodiments, the disclosure provides a vaccine composition or vaccine adjuvant composition comprising a compound of formula I, IA, IB, IC, 1D, or 1E described herein, and prodrugs, analogs and derivatives thereof.

In embodiments, the disclosure provides a pharmaceutical composition comprising a compound of formula I, IA, IB, IC, 1D, or 1E described herein, and prodrugs, analogs and derivatives thereof.

In embodiments, the disclosure provides a method of treating cancer in a subject in need of such treatment, comprising administering to the subject a composition comprising a compound of formula I, IA, IB, IC, 1D, or 1E described herein, and prodrugs, analogs and derivatives thereof. In embodiments, the method further comprises administering to the subject a PD-1/PD-L1 inhibitor or an agonist of an immune co-stimulatory molecule. In embodiments, the the PD-1/PD-L1 inhibitor is selected from the group consisting of nivolumab, pembrolizumab, pidilizumab, BMS-936559, atezolizumab, durvalumab, and avelumab. In embodiments, the agonist of an immune co-stimulatory molecule is an anti-OX40 (CD134) agonist antibody. In accordance with the foregoing methods, the subject may be a human subject and the cancer may be a cancer as described hereinabove. In embodiments, the cancer is a solid tumor. In embodiments, the cancer is refractory.

The disclosure further provides a composition for use in therapy, the composition comprising a compound of formula I, IA, IB, IC, 1D, or 1E described herein, and prodrugs, analogs and derivatives thereof.

The disclosure also provides a composition for use in a method for modulating an immune response in a subject in need of such treatment, the composition comprising a compound of formula I, IA, IB, IC, 1D, or 1E described herein, and prodrugs, analogs and derivatives thereof.

The disclosure also provides a composition for use in a method for treating cancer in a subject in need of such treatment, the composition comprising a compound of formula I, IA, IB, IC, 1D, or 1E described herein, and prodrugs, analogs and derivatives thereof.

The disclosure also provides a composition for use in a method for potentiating an immune response in a subject in need of such treatment, the composition comprising a compound of formula I, IA, IB, IC, 1D, or 1E described herein, and prodrugs, analogs and derivatives thereof.

The disclosure also provides a composition for use in a method for treating a disease or disorder amendable to treatment by activation of NFkB, p38, and JNK cell signaling pathways in cells of a subject in a subject in need of such treatment, the composition comprising a compound of formula I, IA, IB, IC, 1D, or 1E described herein, and prodrugs, analogs and derivatives thereof.

The disclosure also provides a composition for use in treating or preventing a disease or disorder caused by an infectious agent selected from a bacteria, virus, or parasite in a subject in need thereof, the composition comprising a compound of formula I, IA, IB, IC, 1D, or 1E described herein, and prodrugs, analogs and derivatives thereof.

The disclosure also provides a composition for use in a method for treating cancer in a subject in need of such treatment, the composition comprising a compound of formula I, IA, IB, IC, 1D, or 1E described herein, and prodrugs, analogs and derivatives thereof, and the method comprising combination therapy of the ALPK1 agonist with an immune modulator selected from one or more of an inhibitor or antagonist of an immune checkpoint regulator, an immune stimulatory molecule, and an agonist of an immune co-stimulatory molecule.

The disclosure also provides a composition for use in a method for treating a liver disease or disorder in a subject in need of such treatment, the composition comprising a a compound of formula I, IA, IB, IC, 1D, or 1E described herein, and prodrugs, analogs and derivatives thereof, wherein the liver disease or disorder is optionally selected from liver cancer, non-alcoholic steatohepatitis (NASH), and a disease or disorder caused by infection with the hepatitis C virus (HCV) or the hepatitis B virus (HBV).

### BRIEF DESCRIPTION OF THE FIGURES

FIG. 1: Schematic of bacterial H1b-ADP-biosynthetic pathway.

FIGS. 2A-C: Intraperitoneal injection of compound 28 (0.1 mg/kg) led to down-regulation of (A) HBV DNA expression (copy/μl) and to lower levels of (B) HBsAg in serum and (C) HBeAg in serum in the HBV-AAV mouse model after intravenous injection of HBV.

FIG. 3: HBsAg, adw plus compound 28 (injected at a dose of 1 nmol) exhibits an increased IgG response in mice against HBsAg, adw compared to when HBsAg, adw was injected alone. N=8/group.

FIG. 4A-C: Compound 28 (4 nmol and 20 nmol) causes a decrease in the production of (A) eosinophils, (B) dendritic cells and (C) neutrophils when injected intraperitoneally with 30 μg/dose porcine pancreatic elastase (PPE). * p<0.05, ** p<0.01, *** p<0.001, t test.

FIG. 5A-B: RANTES levels at 24 hours (A) or 48 hours (B) post-treatment in C57/B6 mice injected subcutaneously with either PBS as a negative control, or 10 nmole of either Compound 15 or Compound 28, n=3 animals/group. RANTES levels in serum were analyzed by MS Inflammation CBA Kit (BD 552364).

FIG. 6: HEK293 cells were treated with H1b-ADP derivatives (A1, A2, A3, and A4) or with Compound 28 for 4 hours followed by analysis of IL-8 secretion using an IL-8 enzyme linked immunoassay (ELISA). IL-8 secretion is an indication of ALPK1 activation. EC50 values are shown.

FIG. 7A-B: HEK293-NFkB-AP reporter cells were treated with H1b-ADP derivatives A1, A2, A3, A4, (A); or A18, A26, A27, A28 and A30 (B). In each experiment compound 28 was included for reference. Cells were treated with compounds for 7 hours followed by analysis of secreted alkaline phosphatase using para-nitrophenyl phosphate (pNPP) as the substrate. The NF-kB driven alkaline phosphatase expression is an indication of ALPK1 activation. EC50 values are shown. Compound A18 did not have any activity and is not shown in the figure.

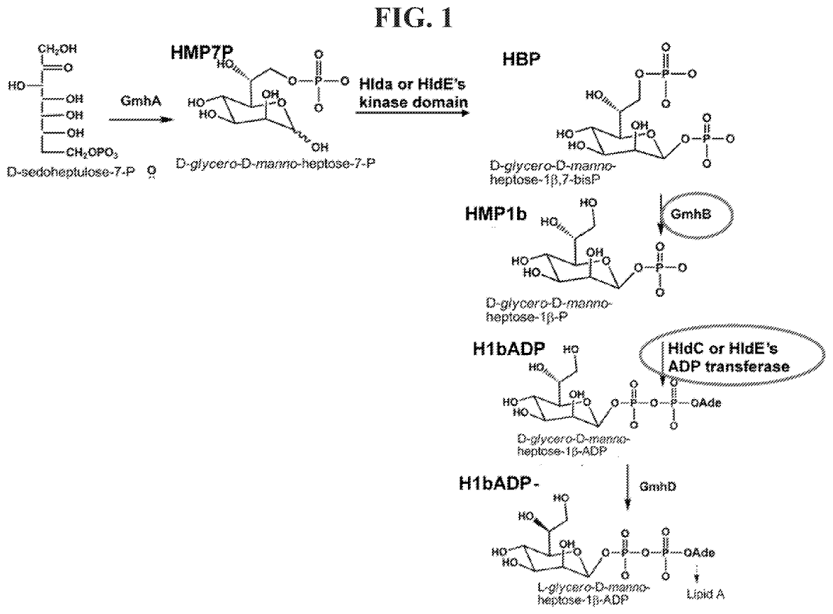

### DETAILED DESCRIPTION

The disclosure provides compounds that are derivatives of certain bacterial metabolites in the ADP-heptose biosynthetic pathway, compositions comprising same, and methods for their use in therapy.

### Definitions

As used herein, the term “ALPK1” may refer to either one of two splice variants, isoform 1 or isoform 2, of the human ALPK1 gene. Each isoform shares the same kinase domain. For reference, the human ALPK1 gene is identified by Entrez Gene ID 80216.

As used herein, the term “activation of ALPK1” refers to the activation of ALPK1 kinase activity. In embodiments, the disclosure provides methods of activating ALPK1 by providing an ALPK1 agonist which may be, for example, an ALPK1 activating ligand, such as HBP, or a prodrug, analog or derivative thereof. Methods for making synthetic HBP are known, for example, as described in Inuki S et al. Organic Letter 2017 19(12):3079-82. In embodiments, the ALPK1 agonist is selected from HMP-1bP and H1b-ADP and prodrugs, analogs and derivatives thereof. In embodiments, the ALPK1 agonist is H1b-ADP, or a prodrug, analog or derivative thereof. In some embodiments, the disclosure provides methods of activating ALPK1 by providing an ALPK1 agonist represented by formula I, IA, IB, IC, 1D, or 1E.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. In some embodiments, alkyl groups are substituted with 1-2 substituents. As a non-limiting example, suitable substituents include halogen and hydroxyl.

As used herein, “alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Alkenyl groups can be substituted or unsubstituted.

As used herein, the term “alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted. In some embodiments, alkylene groups are substituted with 1-2 substituents. As a non-limiting example, suitable substituents include halogen and hydroxyl.

As used herein, the term “alkoxy” or “alkoxyl” refers to an alkyl group having an oxygen atom that connects the alkyl group to the point of attachment: alkyl-O—. As for alkyl group, alkoxyl groups can have any suitable number of carbon atoms, such as C1-6. Alkoxyl groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be substituted or unsubstituted.

As used herein, the term “alkenyloxy” or “alkenyloxyl” refers to an alkenyl group, as defined above, having an oxygen atom that connects the alkenyl group to the point of attachment: alkenyl-O—. Alkenyloxyl groups can have any suitable number of carbon atoms, such as C1-6. Alkenyloxyl groups can be further substituted with a variety of substituents described within. Alkenyloxyl groups can be substituted or unsubstituted.

As used herein, the term “alkylamine” or “alkylamino” refers to an alkyl group having a nitrogen atom that connects the alkyl group to the point of attachment: alkyl-N—. As for alkyl group, alkoxyl groups can have any suitable number of carbon atoms, such as C1-6.

As used herein, the term “halogen” refers to fluorine, chlorine, bromine and iodine.

As used herein, the term “haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, etc.

As used herein, the term “haloalkoxyl” or “haloalkoxy” refers to an alkoxyl group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens.

As used herein, the term “alkanoyl” refers to an alkyl group having a carbonyl group that connects the alkyl group to the point of attachment: alkyl-C(O)—. As for alkyl group, alkanoyloxyl groups can have any suitable number of carbon atoms, such as C1-4. For example, an alkanoyl groups include acetyl, propinoyl, butyryl, etc.

As used herein, the term “alkanoyloxyl” refers to an alkanoyl group having a an oxygen atom that connects the alkanoyl group to the point of attachment: alkyl-C(O)—O—. As for the alkyl group, alkanoyloxyl groups can have any suitable number of carbon atoms, such as C1-4. Exemplary alkanoyloxyl groups include acetoxy, propionyloxy, butryloxy, etc.

As used herein, the term “oxo” refers to an oxygen atom connected to the point of attachment by a double bond (═O).

As used herein, the term “aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. In some embodiments, aryl groups are substituted with 1-2 substituents. As a non-limiting example, suitable substituents include halogen, hydroxyl, —NO2, C1-8 alkyl, C1-8 alkoxy.

As used herein, the term “aralkyloxyl” refers to an aryl group, as defined above, having an alkyl and oxygen atom that connects the aryl group to the point of attachment: aryl-alkyl-O—. As for alkyl group, aralkyloxyl groups can have any suitable number of carbon atoms, such as C1-4.

As used herein, the term “heteroaryl” refers to a monocyclic or fused bicyclic aromatic ring assembly containing 5 to 12 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 9 ring members and from 1 to 4 heteroatoms, or from 5 to 9 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), purine. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

As used herein, “cycloalkyl” refers to a saturated ring assembly containing from 3 to 8 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈. Cycloalkyl rings include, for example, cyclopropyl, cyclobutyl, cyclopentyl, cyclohexyl, and cyclooctyl. Cycloalkyl groups can be substituted or unsubstituted.

As used herein, “heterocyclyl” refers to a saturated ring system having from 3 to 12 ring members and from 1 to 4 heteroatoms of N, O and S. Additional heteroatoms can also be useful, including, but not limited to, B, Al, Si and P. The heteroatoms can also be oxidized, such as, but not limited to, —S(O)— and —S(O)₂—. The N atom can further be substituted to form tertiary amine or ammonium salts. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, tetrahydrofuran, oxane (tetrahydropyran), oxepane, thiolane (tetrahydrothiophene), thiane (tetrahydrothiopyran), oxazolidine, isoxazolidine, thiazolidine, isothiazolidine, dioxolane, dithiolane, morpholine, etc. Heterocycloalkyl groups can be unsubstituted or substituted. For example, heterocycloalkyl groups can be substituted with C₁₋₆ alkyl or oxo (═O), among many others.

Certain compounds of the present invention possess asymmetric carbon atoms (optical centers) or double bonds; the racemates, diastereomer, geometric isomers, regioisomers and individual isomers (e.g., separate enantiomers) are all intended to be encompassed within the scope of the present invention. In some embodiments, the compounds of the present invention are a particular enantiomer, anomer, or diastereomer substantially free of other forms.

Certain compounds of the present disclosure include one or more thiophosphate moieties. The current disclosure generally displays the thiophosphate moiety as

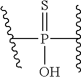 [SMILES: *P(*)(O)=S]

However, a person of skill in the art will recognize that the thiophosphate moiety can interconvert to

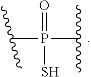 [SMILES: *P(*)(=O)S]

All stable interconversions of the thiophosphate moieties of the present disclosure are within the scope of this application.

As used herein, the term “substantially free” refers to an amount of 10% or less of another form, preferably 8%, 5%, 4%, 3%, 2%, 1%, 0.5%, or less of another form. In some embodiments, the isomer is a stereoisomer.

### Detailed Description of the Embodiments

The present disclosure provides compounds represented by formula (I) compound represented by Formula I, or a stereoisomer, tautomer, stable isotope, prodrug or pharmaceutically acceptable salt thereof:

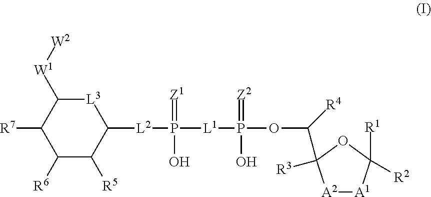

wherein

A¹ and A² are independently selected from O, S and —C(R⁸R⁹)—, wherein R⁸ and R⁹ are independently selected from H, D, —OH, N₃, —CN, halogen and an optional substituted group selected C1-C4 alkyl, C1-C4 alkoxy, C1-C4 haloalkyl, C1-C4 haloalkoxy, C1-C4 alkanoyloxy, C1-C4 alkenyloxy and aralkyloxy, wherein the optional substituents are 1-3 substituents independently selected from D, halogen, —OH, ═O, C1-C4 alkyl and C1-C4 alkoxy; at least one of A¹ or A² is —C(R⁸R⁹); wherein R⁸ or R⁹ in A¹ can cyclize with R⁸ or R⁹ in A² to form C3-C6 cycloalkyl and heterocyclyl containing 3 to 9 ring members and having 1-3 heteroatoms selected from N, O and S as ring members, each optionally substituted by 1-3 substituents independently selected from D, halogen, —OH, ═O, C1-C4 alkyl and C1-C4 alkoxy;

L¹ and L² are independently selected from O, CH₂, CHF and CF₂;

L³ is O, S, CH₂ or CH(OH);

Z¹ and Z² are independently selected from O and S;

W¹ is —C(R¹⁰R¹¹)—, wherein R¹⁰ and R¹¹ are independently selected from H, D, —OH, halogen, and optionally substituted groups selected from C1-C4 alkyl, C1-C4 alkoxy, C1-C4 haloalkyl, C1-C4-haloalkoxy, C1-C4 alkenyloxy, aralkyloxy, 1-6 membered oligopeptidyl linked via C-terminal C(O)O—; and R¹²CO₂—, wherein R¹² is selected from C1-C20 alkyl, C1-C20 alkenyl, C1-C20 alkoxy, C1-C20 alkenyloxy, C1-C20 alkylamino, C3-C6 cycloalkyl, heterocyclyl containing 4 to 6 ring members and having 1-3 heteroatoms selected from N, O and S as ring members, aryl, and heteroaryl containing 5 to 10 ring atoms and having 1-3 heteroatoms selected from N, O and S as ring members and 1-6 membered oligopeptidyl linked via N-terminal N; wherein the optional substituents for R¹⁰ and R¹¹ are 1-3 substituents independently selected from D, halogen, —OH, ═O, C1-C4 alkyl and C1-C4 alkoxy;

and

when W¹ is —C(R¹⁰R¹¹)—, wherein R¹⁰ is F, and the others are defined as above, W² is H or C1-C3 alkyl optionally substituted with 1-3 substituents independently selected from D, halogen, —OH, ═O, C1-C3 alkoxyl, C1-C3 haloalkyl, C1-C3 haloalkoxyl, C1-C3 alkenyloxyl and R¹²CO₂—, wherein R¹² is C1-C4 alkyl, C1-C4 alkoxy, C1-C4 alkylamino, C3-C6 cycloalkyl, cycloheteroalkyl containing 3 to 6 ring members and having 1-3 heteroatoms selected from N, O and S as ring members, C6-C10 aryl, and heteroaryl containing 5 to 10 ring atoms and having 1-3 heteroatoms selected from N, O and S as ring members;

W² is R¹³-Q¹-W³—, wherein Q¹ is selected from —O— or —NH—; W³ is selected from a bond or C1-C3 alkylene groups optionally substituted with 1-3 substituents independently selected from halogen, —OH, C1-C3 alkoxy, C1-C3 haloalkyl, C1-C3 haloalkoxy, C1-C3 alkenyloxy; wherein R¹³ is 1-6 membered oligopeptidyl linked via C-terminal carbonyl group or R¹⁴Q²C(O)—; wherein Q² is a bond, —O— or —NH—; R¹⁴ is 1-6 membered oligopeptidyl linked via N-terminal N or an optionally substituted group selected from C5-C20 alkyl, C5-C20 alkylenyl and R¹⁵-Q³-Q⁴-Q⁵-; wherein Q³,Q⁴ and Q⁵ are independently selected from a bond, aryl, heteroaryl containing 5 to 6 ring atoms, C3-C6 cycloalkyl and heterocyclyl containing 4 to 6 ring members and having 1-3 heteroatoms selected from N, O and S as ring members, and at least one of Q³, Q⁴ and Q⁵ is not a bond; R¹⁵ is an optionally substituted group selected from C1-C18 alky and C1-C18 alkoxy, wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy;

R¹ is aryl or heteroaryl containing 6 to 10 ring atoms and having 1-4 heteroatoms selected from N, O and S as ring members, wherein R¹ is optionally substituted with 1-3 substituents selected from D, halogen, —OH, ═O, CN, NH₂ and an optionally substituted group selected from C1-C4 alkyl, C1-C4 alkoxy, (R¹⁶R¹⁷)N— and (R¹⁶R¹⁷)NCO—, wherein R¹⁶ and R¹⁷ are independently selected from H and an optionally substituted group selected from C1-C4 alkyl, C3-C6 cycloalkyl, heterocyclyl containing 4 to 6 ring members and having 1-3 heteroatoms selected from N, O and S as ring members, aryl, aralkyl, heteroaryl containing 5 to 10 ring atoms and having 1-3 heteroatoms selected from N, O and S as ring members; and heteroarylalkyl containing 5 to 10 ring atoms and having 1-3 heteroatoms selected from N, O and S as ring members; wherein the optionally substituents are 1-3 substituents independently selected from from D, halogen, —OH, ═O, CN, NH₂ and an C1-C4 alkyl, C1-C4 alkoxy, C1-C3 haloalkyl, C1-C3 haloalkoxy and C3-C6 cycloalkyl;

R², R³ and R⁴ are independently selected from H, D, halogen, C1-C4 alkyl and C1-C4 haloalkyl;

R⁵, R⁶ and R⁷ are independently selected from H, —OH, halogen, and R¹²CO₂—, and at least two of R⁵, R⁶ and R⁷ are —OH or R¹²CO₂—, wherein R¹² is selected from C1-C4 alkyl, C1-C4 alkoxy, C1-C4 alkenyloxy, C1-C4 alkylamino, C3-C6 cycloalkyl, heterocyclyl containing 4 to 6 ring members and having 1-3 heteroatoms selected from N, O and S as ring members, C6-C10 aryl, and heteroaryl containing 5 to 10 ring atoms and having 1-3 heteroatoms selected from N, O and S as ring members; wherein any two of the adjacent groups of R⁵, R⁶ and R⁷ can cyclize to form heterocyclyl containing 5 to 9 ring members and having 1-3 heteroatoms selected from N, O and S as ring members, each optionally substituted by 1-3 substituents independently selected from D, halogen, —OH, ═O, C1-C4 alkyl and C1-C4 alkoxy.

In some embodiments, the compound of formula I is represented by the compound of formula IA and/or a stereoisomer, a stable isotope, prodrug or a pharmaceutically acceptable salt thereof:

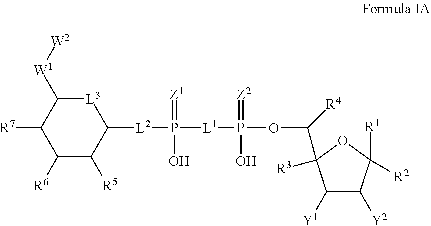

wherein: Y¹ and Y² are independently selected from H, D, —OH, N₃, —CN, halogen and optionally substituted groups selected from C1-C4 alkyl, C1-C4 alkoxy, C1-C4 haloalkyl, C1-C4 haloalkoxy, C1-C4 alkanoyloxy, C1-C4 alkenyloxy and aralkyloxy; wherein the optional substituents are 1-3 substituents independently selected from D, halogen, —OH, ═O, C1-C4 alkyl and C1-C4 alkoxy; and R¹-R⁷, L¹-L³, Z¹, Z², W¹ and W² are defined above.

In some embodiments, Y¹ and Y² in the compound of formula IA are independently selected from H, D, —OH, halogen, C1-C4 alkyl, C1-C4 alkoxy, C1-C4 haloalkyl, C1-C4 haloalkoxy, C1-C4 alkanoyloxy, and C1-C4 alkenyloxy; and R¹-R⁷, L¹-L³, Z¹, Z², W¹ and W² are as defined above.

In some embodiments, Y¹ and Y² in the compound of formula IA are independently selected from —OH, halogen, C1-C4 alkyl, and C1-C4 alkanoyloxy; and R¹-R⁷, L¹-L³, Z¹, Z², W¹ and W² are defined above.

In some embodiments, the compound of formula I is represented by the compound of formula IB and/or a stereoisomer, a stable isotope, prodrug or a pharmaceutically acceptable salt thereof:

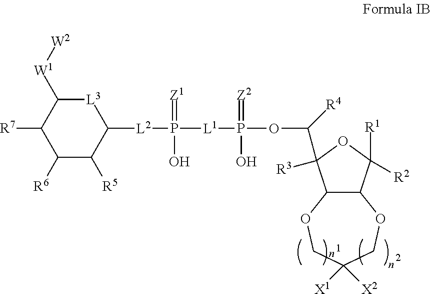

wherein:

n¹ and n² are each an integer independently selected from the group consisting of 0-2;

X¹ and X² are independently selected from H, D, —OH, N₃, —CN, halogen and optionally substituted groups selected from C1-C4 alkyl, C1-C4 alkoxy, C1-C4 haloalkyl, C1-C4 haloalkoxy, C1-C4 alkanoyloxy, C1-C4 alkenyloxy and aralkyloxy, wherein the optional substituents are 1-3 substituents independently selected from D, halogen, —OH, ═O, C1-C4 alkyl and C1-C4 alkoxy; and R¹-R⁷, L¹-L³, Z¹, Z², W¹ and W² are defined above.

In some embodiments, n¹ and n² of formula IB are each 0.

In some embodiments, X¹ and X² of formula IB are independently selected from H, D, C1-C4 alkoxy and C1-C4 alkyl; and R¹-R⁷, L¹-L³, Z¹, Z², W¹ and W² are defined above.

In some embodiments, the compound of Formula I is represented by the compound of formula IC and/or a stereoisomer, a stable isotope, prodrug or a pharmaceutically acceptable salt thereof:

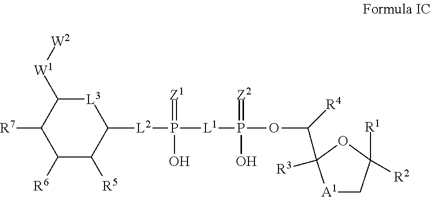

wherein:

A¹ is —C(R¹⁰R¹¹)—, O or S; and R¹-R⁹, L¹-L³, Z¹, Z², W¹ and W² are defined above.

In some embodiments, R², R³, and R⁴ in formulas I, IA, IB, and IC are each H.

In some embodiments, R⁵, R⁶, and R⁷ in formulas I, IA, IB, and IC are each independently selected from the group consisting of —OH, and C1-C4 alkanoyloxy -.

In some embodiments in formulas I, IA, IB, and IC, L³ is O.

In some embodiments in formulas I, IA, IB, and IC, L² is O.

In some embodiments in formulas I, IA, IB, and IC, L¹ is O or S.

In some embodiments, W¹ in formulas I, IA, IB, and IC is —C(R¹⁰R¹¹)—, wherein R¹⁰ and R¹¹ are independently selected from H, D, —OH, halogen, C1-C4 alkyl, C1-C4 alkoxy, C1-C4 haloalkyl, C1-C4-haloalkoxy, C1-C4 alkanoyloxy, C1-C4 alkenyloxy, R¹²CO₂—, wherein R¹² is selected from C1-C20 alkyl, C1-C20 alkoxy, C1-C20 alkanoyloxy and C1-C20 alkenyloxy.

In some embodiments, W¹ in formulas I, IA, IB, and IC is —C(R¹⁰R¹¹)—, wherein R¹⁰ and R¹¹ are independently selected from H, D, —OH, halogen and C1-C10 alkanoyloxy.

In some embodiments, W² in formulas I, IA, IB, and IC is R¹³-Q¹-W³—, wherein Q¹ is —O—; W³ is a C1 alkylene group and R¹³ is 1-6 membered oligopeptidyl linked via C-terminal carbonyl group;

In some embodiments, W² in formulas I, IA, IB, and IC is R¹³-Q¹-W³—, wherein Q¹ is —O—; W³ is a C1 alkylene group and R¹³ is R¹⁴Q²C(O)—; wherein R¹⁴ is 1-6 membered oligopeptidyl linked via N-terminal group, Q² is a bond;

In some embodiments, W² in formulas I, IA, IB, and IC is R¹³-Q¹-W³—, wherein Q¹ is —O—; W³ is a C1 alkylene group and and R¹³ is R¹⁴Q²C(O)—; wherein R¹⁴ is an optionally substituted group selected from C5-C20 alkyl and C5-C20 alkylenyl, Q² is a bond;

In some embodiments, W² in formulas I, IA, IB, and IC is R¹³-Q¹-W³—, wherein Q¹ is —O—; W³ is a C1 alkylene group and R¹³ is R¹⁴Q²C(O)—; Q² is a bond, wherein R¹⁴ is an optionally substituted group selected from R¹⁵-Q³-Q⁴-Q⁵-; wherein Q³,Q⁴ and Q⁵ are independently selected from a bond, aryl, heteroaryl containing 5 to 6 ring atoms, C3-C6 cycloalkyl and heterocyclyl containing 4 to 6 ring members and having 1-3 heteroatoms selected from N, O and S as ring members, and at least one of Q³, Q⁴ and Q⁵ is not a bond; R¹⁵ is an optionally substituted group selected from C1-C18 alky and C1-C18 alkoxy, wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy;

In some embodiments, W² in formulas I, IA, IB, and IC is R¹³-Q¹-W³—, wherein Q¹ is —O—; W³ is a C1 alkylene group and R¹³ is R¹⁴Q²C(O)—; Q² is a bond, wherein R¹⁴ is an optionally substituted group selected from R¹⁵-Q³-Q⁴-Q⁵-; wherein Q³,Q⁴ and Q⁵ are independently selected from a bond, aryl, and at least one of Q³, Q⁴ and Q⁵ is not a bond; R¹⁵ is an optionally substituted group selected from C1-C18 alky and C1-C18 alkoxy, wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy;

In some embodiments, W² in formulas I, IA, IB, and IC is R¹³-Q¹-W³—, wherein Q¹ is —O—; W³ is a C1 alkylene group and R¹³ is R¹⁴Q²C(O)—; wherein R¹⁴ is an optionally substituted group selected from C5-C20 alkyl group and Q² is a bond;

In some embodiments, R¹ in formulas I, IA, IB, and IC is selected from

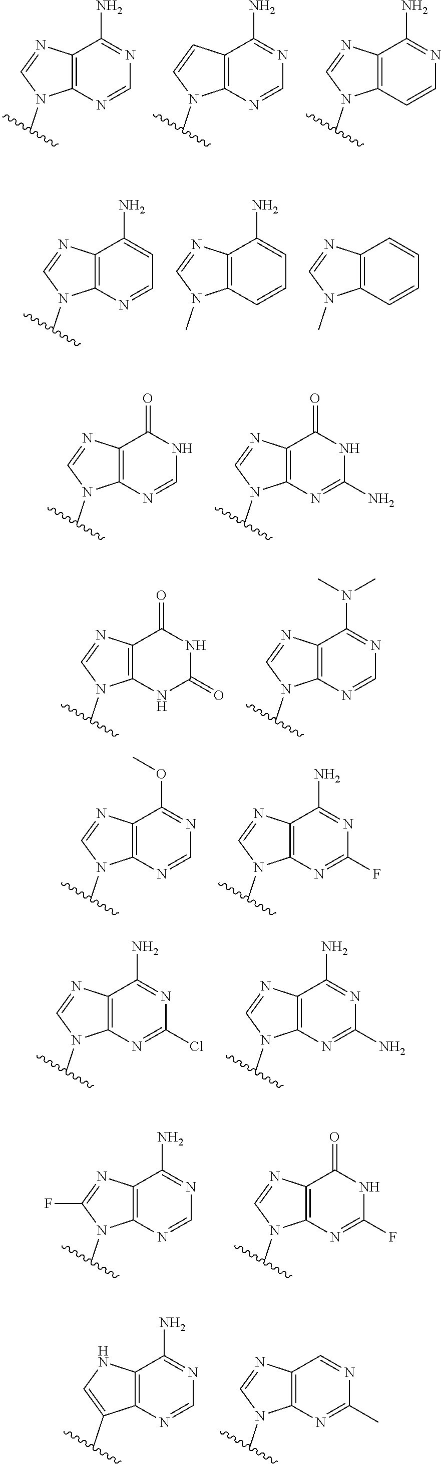

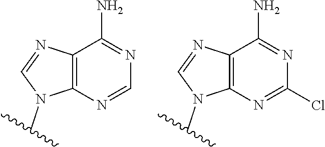 [SMILES: *n1cnc2c(N)ncnc21 ; *n1cnc2c(N)nc(Cl)nc21]

In some embodiments, R¹ in formulas I, IA, IB, and IC is selected from

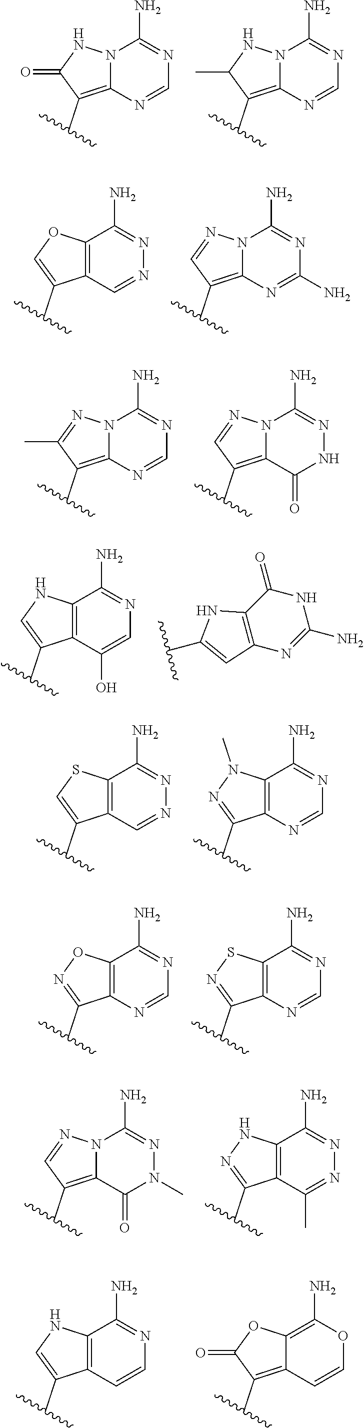

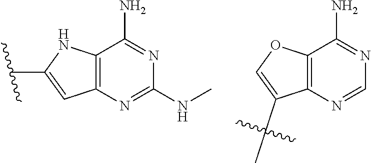 [SMILES: *c1cc2nc(NC)nc(N)c2[nH]1 ; *c1coc2c(N)ncnc12]

In some embodiments, R¹ in formulas I, IA, IB, and IC is selected from

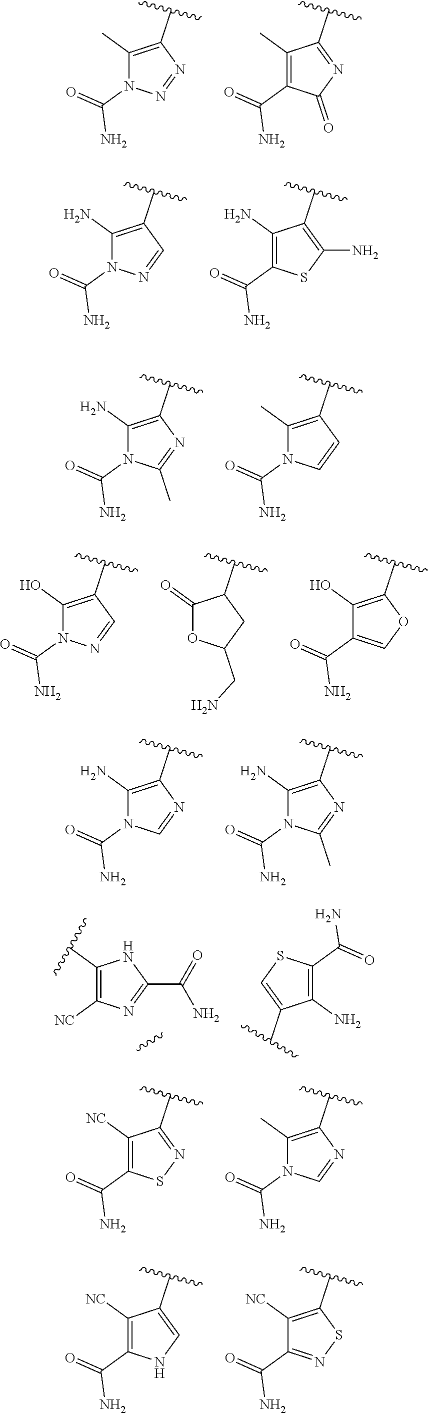

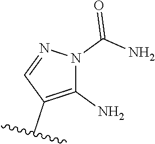 [SMILES: *c1cnn(C(N)=O)c1N]

In some embodiments, R¹ in formulas I, IA, IB, and IC is selected from

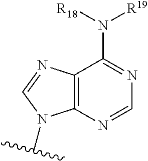

wherein

R¹⁸ and R¹⁹ are independently selected from H and an optionally substituted group selected from heterocyclyl C6-C10 arylalkyl, heteroarylalkyl containing 5 to 10 ring atoms and having 1-3 heteroatoms selected from N, O and S as ring members, R²⁰CO— and R²¹S(O)₂—; wherein the substitutents for 1-3 substituents independently selected from halogen, OH, ═O, CN, NH₂, C1-C3 alkyl, C1-C3 haloalkyl, C1-C3 alkyloxy, C1-C3 haloalkyloxy, C3-C6 cycloalkyl, C3-C6 cycloalkyloxy, heterocyclyl containing 4 to 6 ring members and having 1-3 heteroatoms selected from N, O and S as ring members, C6-C10 aryl, C6-C10 haloaryl, heteroaryl containing 5 to 10 ring atoms and having 1-3 heteroatoms selected from N, O and S as ring members, and haloheteroaryl containing 5 to 10 ring atoms and having 1-3 heteroatoms selected from N, O and S as ring members; R²⁰ and R²¹ are C1-C20 alkyl, C1-C20 alkenyl, heterocyclyl containing 4 to 6 ring members and having 1-3 heteroatoms selected from N, O and S as ring members, C6-C10 aryl, C6-C10 haloaryl, heteroaryl containing 5 to 10 ring atoms and having 1-3 heteroatoms selected from N, O and S as ring members, and haloheteroaryl containing 5 to 10 ring atoms and having 1-3 heteroatoms selected from N, O and S as ring members.

In some embodiments, R¹ in formulas I, IA, IB, and IC is selected from

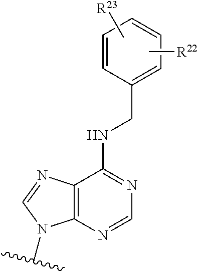

wherein R²² and R²³ are independently selected from H, D halogen, C1-C4 alkyl, C1-C4 alkyloxy, C1-C4 haloalkyl, C1-C4 haloalkyloxy, C1-C4-cycloalkyl and C1-C4-cycloalkyloxy.

In some embodiments, the compound of Formula I, is represented by the compound of Formula ID and/or a stereoisomer, a stable isotope, prodrug or a pharmaceutically acceptable salt thereof:

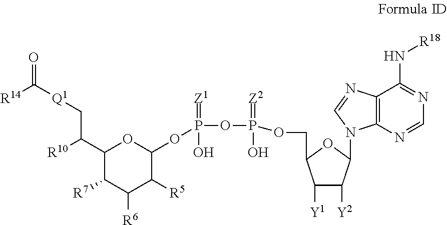

wherein R⁵-R⁷, R¹⁰, R¹⁴, R¹⁸, Q, Y1, Y², Z¹ and Z² are as defined above.

In some embodiments, the compound of Formula I, is represented by the compound of Formula IE and/or a stereoisomer, a stable isotope, prodrug or a pharmaceutically acceptable salt thereof:

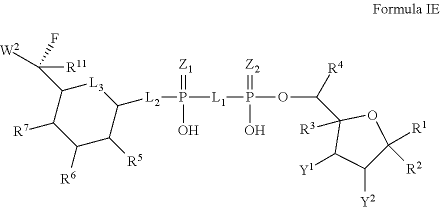

wherein

Z¹ and Z² are independently selected from O and S wherein at least one of Z¹ and Z² is S;

W² is H or C1-C3 alkyl optionally substituted with 1-3 substituents independently selected from D, halogen, —OH, ═O, C1-C3 alkoxyl, C1-C3 haloalkyl, C1-C3 haloalkoxyl, C1-C3 alkenyloxyl and R¹²CO₂—, wherein R¹² is C1-C4 alkyl, C1-C4 alkoxy, C1-C4 alkylamino, C5-C20 alkyl, C5-C20 alkenyl, C5-C20 alkoxy, C5-C20 alkenyloxy, C5-C20 alkylamino, C3-C6 cycloalkyl, cycloheteroalkyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members; and R¹-R⁷, R¹¹, L¹-L³, Y¹, Y², and W² are defined above.

In embodiments of Formula IE, Z² is S and Z¹ is O.

In embodiments of Formula IE, Z² is S and Z¹ is S.

In embodiments of Formula IE, R², R³, and R⁴ are each H.

In embodiments of Formula IE, R⁵, R⁶, and R⁷ are each independently selected from the group consisting of —OH, and C1-C4 alkanoyloxyl.

In embodiments of Formula IE, L³ is O.

In embodiments of Formula IE, L² is O.

In embodiments of Formula IE, L¹ is O.

In embodiments of Formula IE, R¹¹ is selected from H, D, —OH, halogen, C1-C4 alkyl, C1-C4 alkoxyl, C1-C4 haloalkyl, C1-C4-haloalkoxyl, C1-C4 alkanoyloxyl, C1-C4 alkenyloxyl and R¹²CO₂—, wherein R¹² is selected from C1-C4 alkyl, C1-C4 alkoxyl, C1-C4 alkanoyloxyl and C1-C4 alkenyloxyl.

In embodiments of Formula IE, R¹¹ is selected from H, D, —OH, and halogen.

In embodiments of Formula IE, R¹¹ is H.

In embodiments of Formula IE, W² is C1-C3 alkyl optionally substituted with 1-3 substituents independently selected from D, halogen, —OH, ═O and C1-C3 alkoxyl, C1-C3 haloalkyl, C1-C3 haloalkoxyl, C1-C3 alkenyloxyl and R¹²CO₂—, wherein R¹² is C1-C alkyl, C1-C4 alkoxy and C1-C4 alkylamino.

In embodiments of Formula IE, W² is C1-C3 alkyl optionally substituted with 1-3 substituents independently selected from D, halogen, —OH and R¹²CO₂—, wherein R¹² is C1-C3 alkyl.

In embodiments of Formula IE, W² is C1 alkyl optionally substituted with 1 substituent selected from —OH and R¹²CO₂—, wherein R¹² is C1-C3 alkyl.

In embodiments, when W² is R¹²CO₂—, R¹² is selected from C5-C20 alkyl, C5-C20 alkenyl, C5-C20 alkoxy, C5-C20 alkenyloxy, C5-C20 alkylamino, and R¹ in formulas IE is selected from

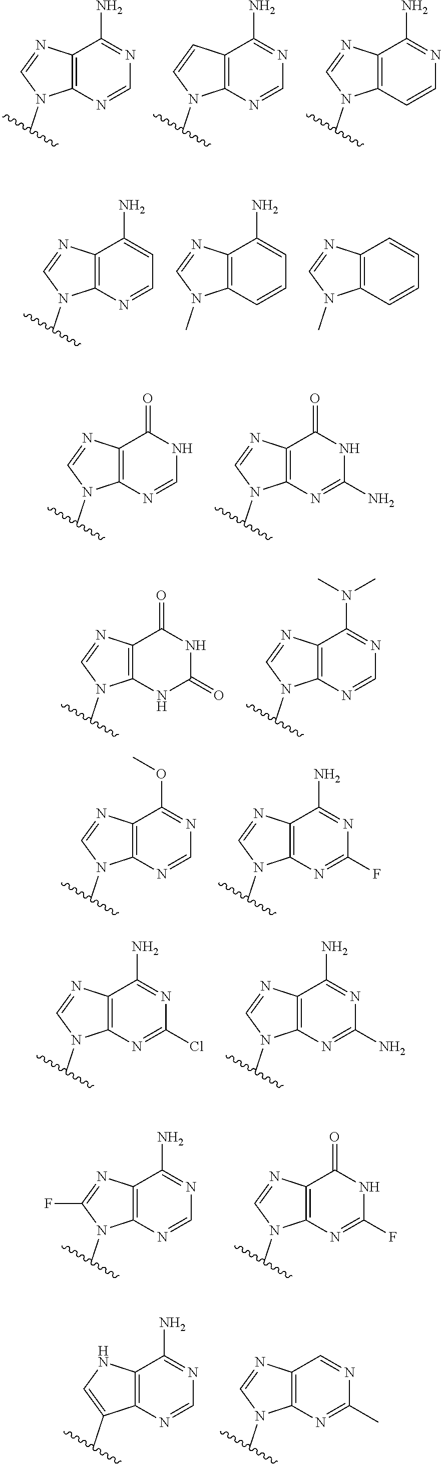

In embodiments, R¹ in formulas IE is selected from

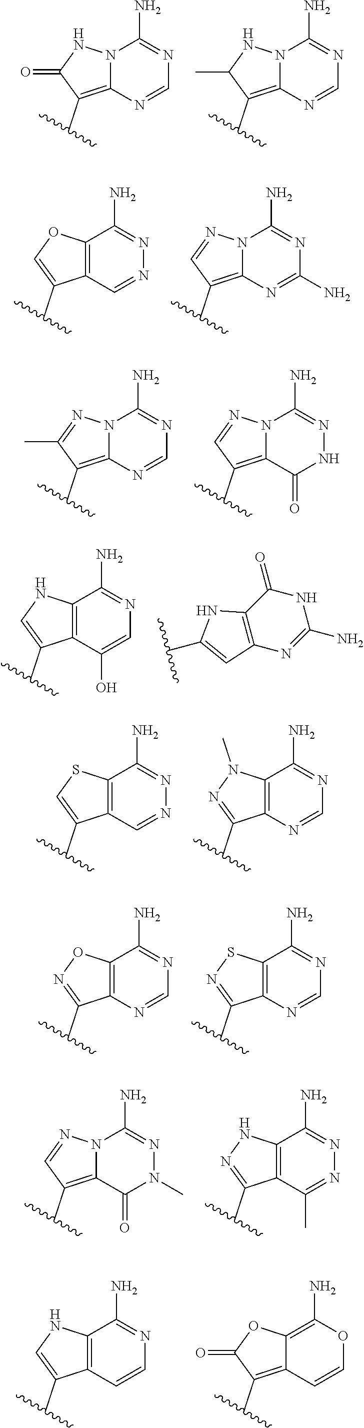

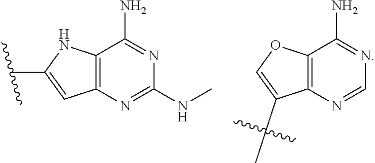 [SMILES: *c1cc2nc(NC)nc(N)c2[nH]1 ; *c1coc2c(N)ncnc12]

In embodiments, R¹ in formulas IE is selected from

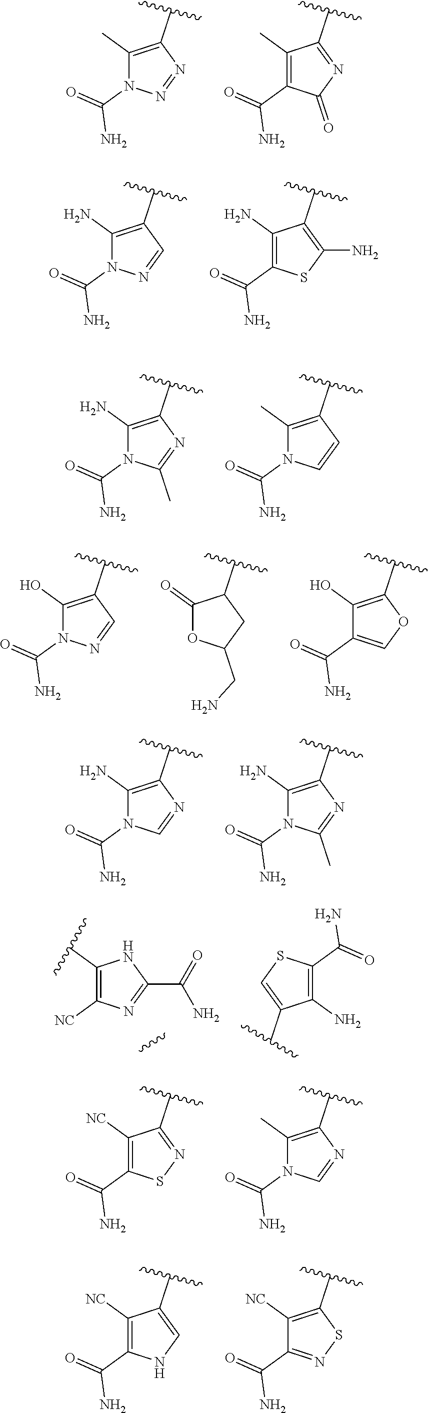

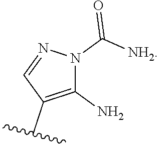 [SMILES: *c1cnn(C(N)=O)c1N]

In embodiments, R¹ in formulas IE is selected from

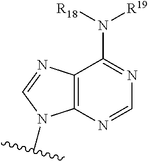

wherein

R¹⁸ and R¹⁹ are independently selected from H and an optionally substituted group selected from heterocyclyl C6-C10 arylalkyl, heteroarylalkyl containing 5 to 10 ring atoms and having 1-3 heteroatoms selected from N, O and S as ring members, R²⁰CO— and R²¹S(O)₂—; wherein the substitutents for 1-3 substituents independently selected from halogen, OH, ═O, CN, NH₂, C1-C3 alkyl, C1-C3 haloalkyl, C1-C3 alkyloxy, C1-C3 haloalkyloxy, C3-C6 cycloalkyl, C3-C6 cycloalkyloxy, heterocyclyl containing 4 to 6 ring members and having 1-3 heteroatoms selected from N, O and S as ring members, C6-C10 aryl, C6-C10 haloaryl, heteroaryl containing 5 to 10 ring atoms and having 1-3 heteroatoms selected from N, O and S as ring members, and haloheteroaryl containing 5 to 10 ring atoms and having 1-3 heteroatoms selected from N, O and S as ring members; R²⁰ and R²¹ are C1-C20 alkyl, C1-C20 alkenyl, heterocyclyl containing 4 to 6 ring members and having 1-3 heteroatoms selected from N, O and S as ring members, C6-C10 aryl, C6-C10 haloaryl, heteroaryl containing 5 to 10 ring atoms and having 1-3 heteroatoms selected from N, O and S as ring members, and haloheteroaryl containing 5 to 10 ring atoms and having 1-3 heteroatoms selected from N, O and S as ring members;

In embodiments, R¹ in formulas IE is selected from

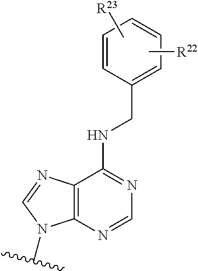

wherein R²² and R²³ are independently selected from H, D halogen, C1-C4 alkyl, C1-C4 alkyloxy, C1-C4 haloalkyl, C1-C4 haloalkyloxy, C1-C4-cycloalkyl and C1-C4-cycloalkyloxy.

In some embodiments, when W² is R¹²CO₂—, R¹² is selected from C5-C20 alkyl, C51-C20 alkenyl, C5-C20 alkoxy, C5-C20 alkenyloxy, C5-C20 alkylamino, and R¹ in formulas IE is

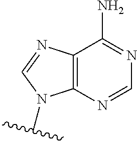 [SMILES: *n1cnc2c(N)ncnc21]

In embodiments, Y¹ and Y² are independently selected from H, D, —OH, halogen, C1-C4 alkyl, C1-C4 alkoxyl, C1-C4 haloalkyl, C1-C4 haloalkoxyl, C1-C4 alkanoyloxyl and C1-C4 alkenyloxyl.

In embodiments, Y¹ and Y² are independently selected from —OH, halogen, C1-C4 alkyl and C1-C4 alkanoyloxyl.

In embodiments, Y¹ and Y² are each —OH.

In embodiments, the compound of Formula I is selected from

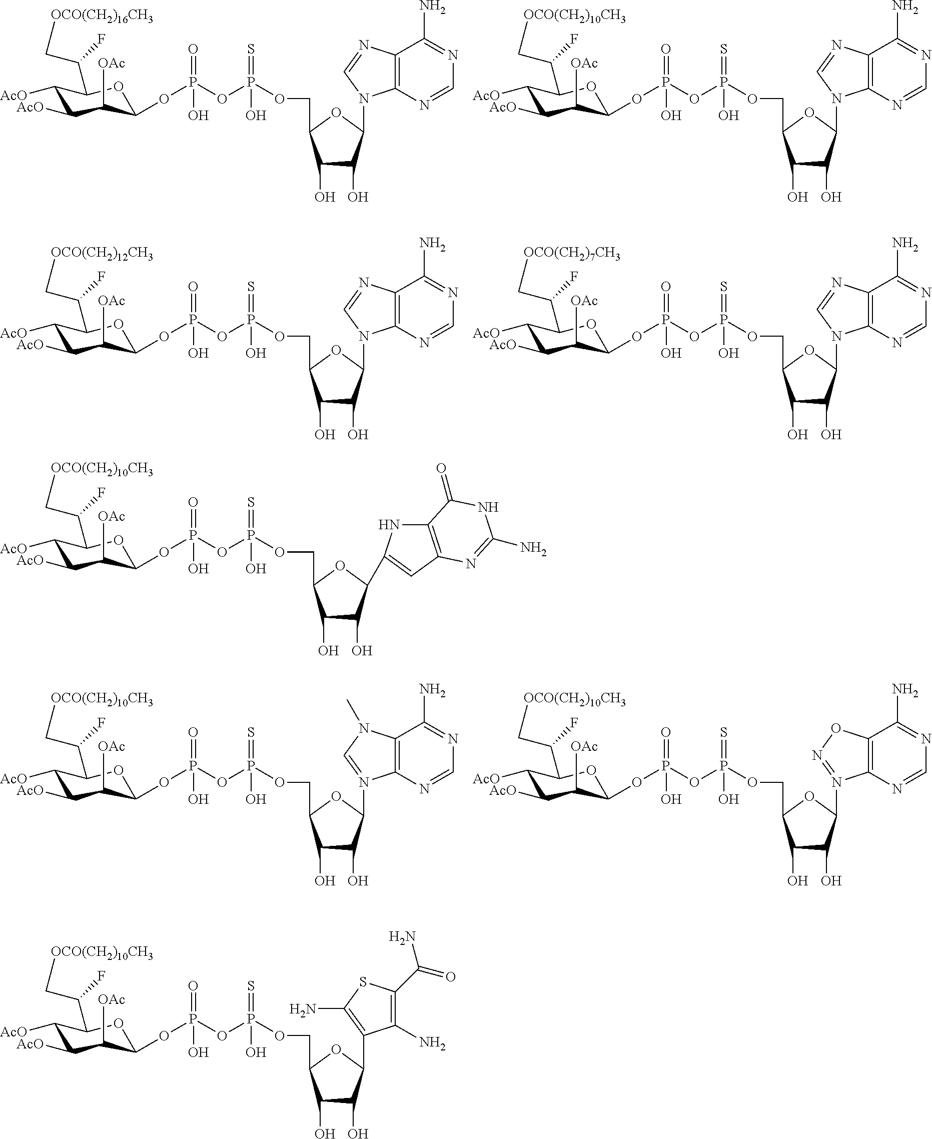

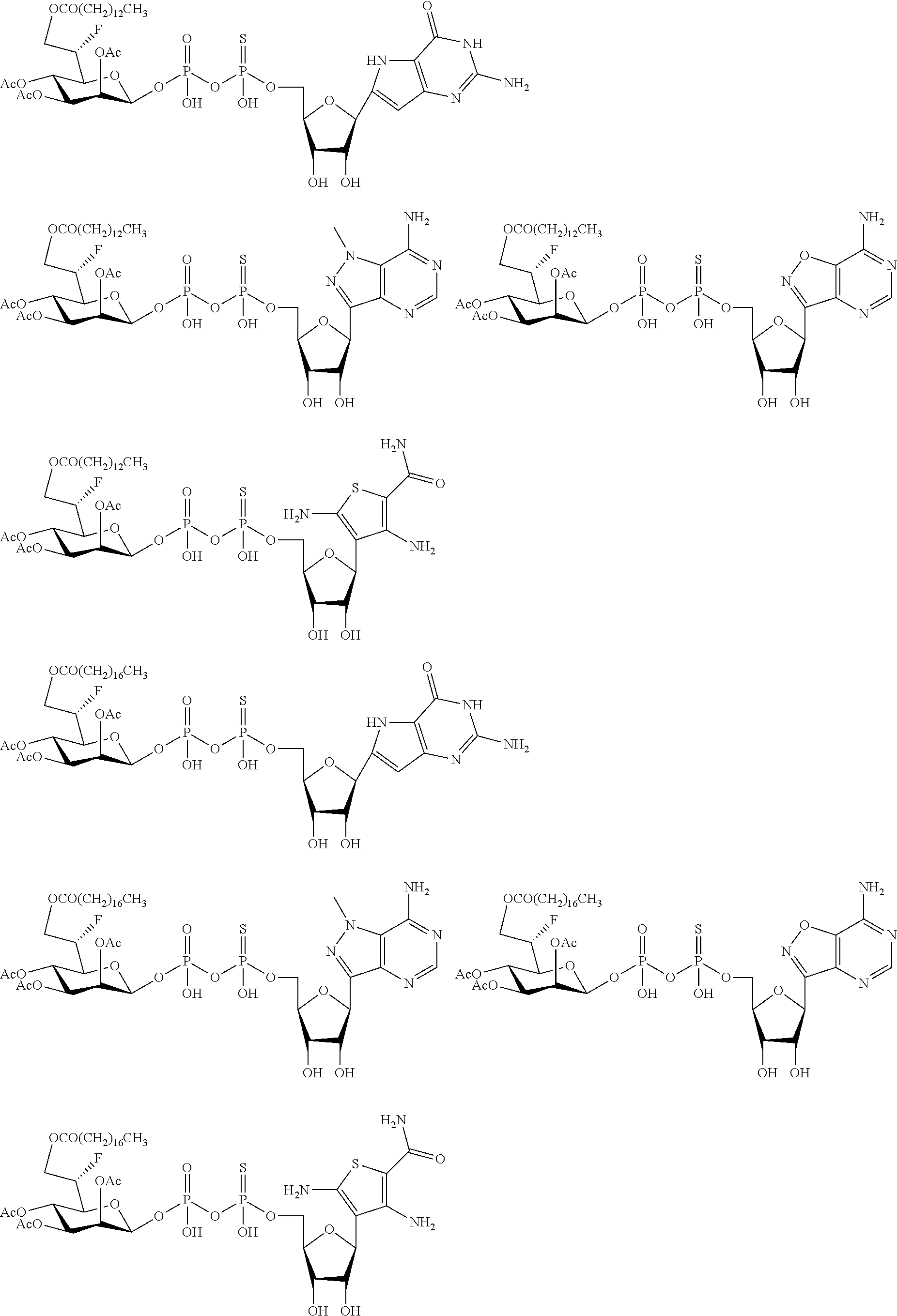

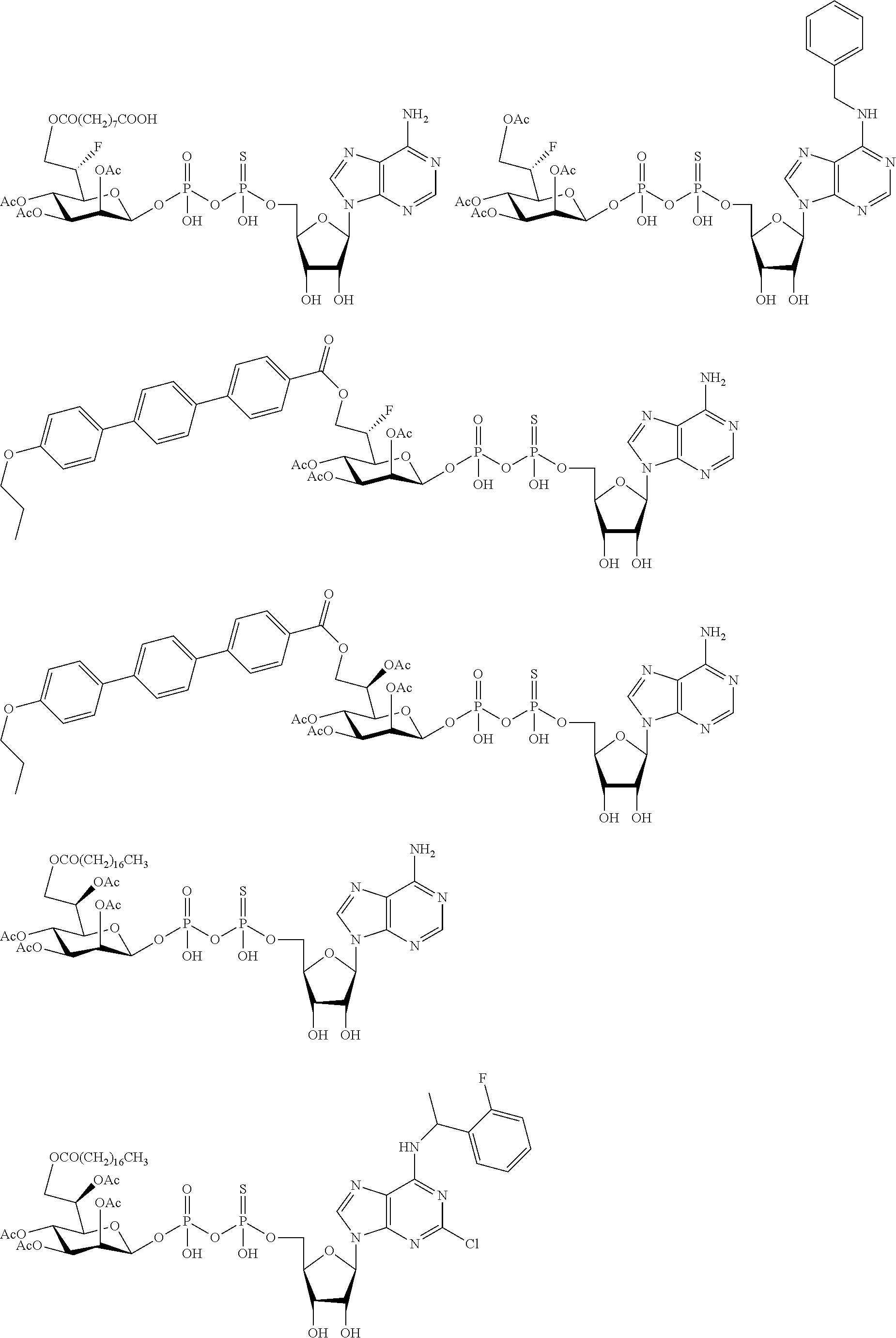

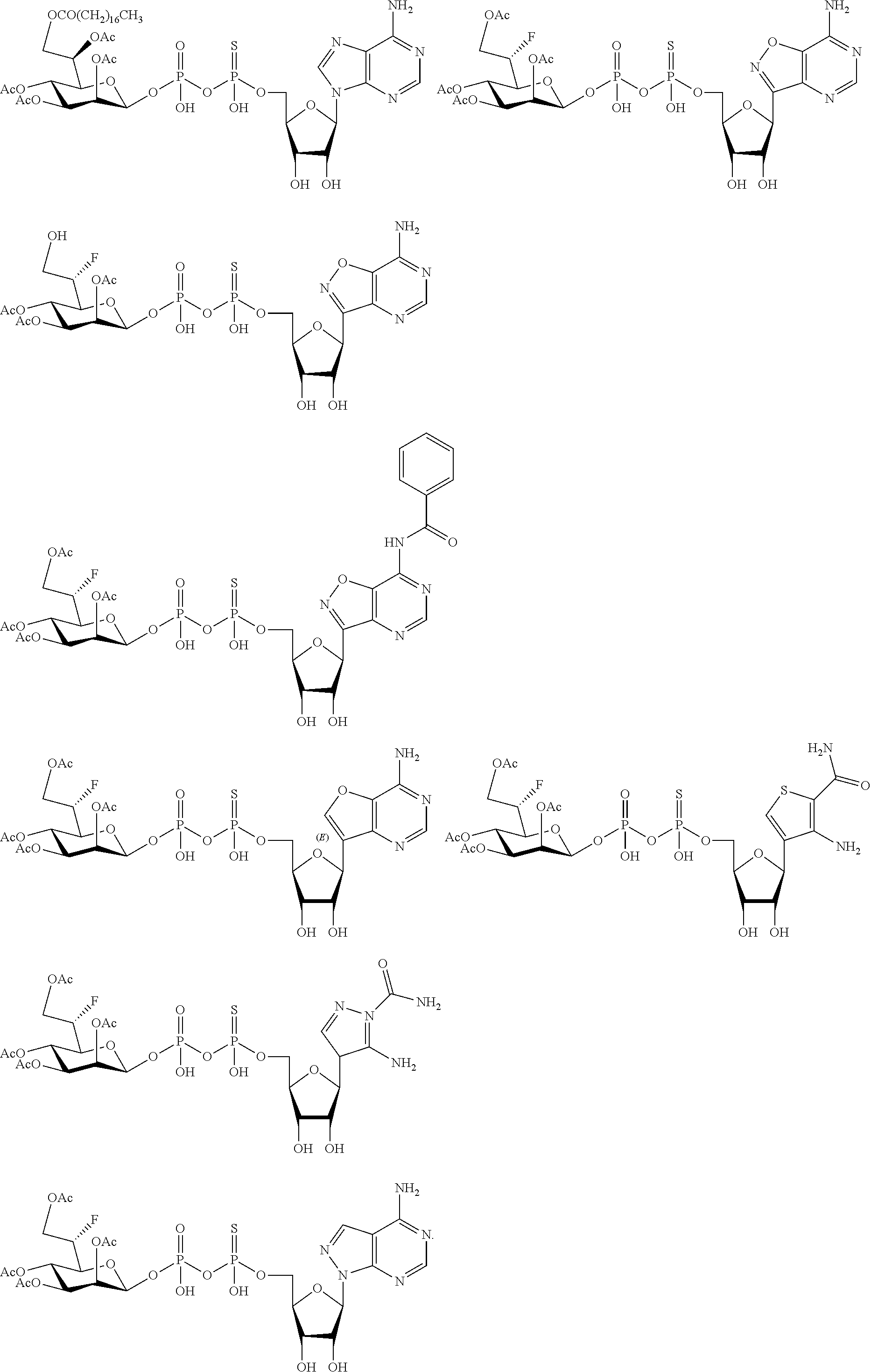

In embodiments, the compound of formula IE is selected from

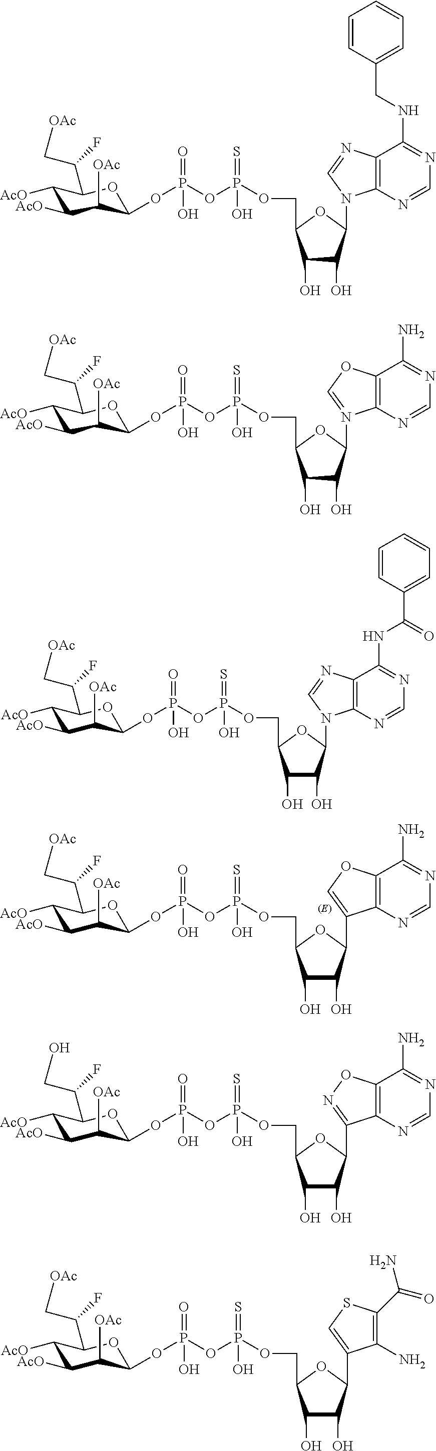

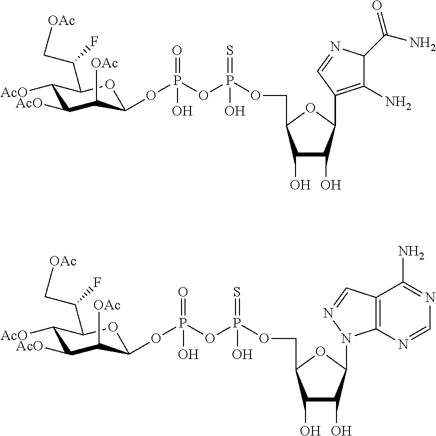

In embodiments, the compound of formula I is a compound described in the Examples of this application.

The compound of the present disclosure can be prepared using the general processes describes in Schemes I, II, III, IV, V, VI and VII as well as the techniques described in the exemplary embodiments.

In embodiments, the disclosure provides a pharmaceutical composition comprising a compound of formula 1 as described herein, and a pharmaceutically acceptable carrier.

In some embodiments, the biological activity of a compound of formula I described herein is improved with respect to a reference compound. In some embodiments, the reference compound is D-glycero-D-manno-heptose-10-ADP (also referred to herein as H1BADP or H1B-D-ADP), which has the following structure

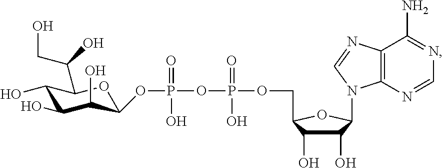

or its diastereomer L-glycero-D-manno-heptose-1l-ADP (also referred to herein as H1B-ADP-6L or H1B-L-ADP).

In embodiments, the disclosure provides an ALPK1 agonist in the form of a compound of formula I described herein, and prodrugs, analogs and derivatives thereof.

In embodiments, the disclosure provides methods of treating cancer by administering a compound of formula I described herein, and prodrugs, analogs and derivatives thereof. In further embodiments of the methods of treating cancer, the disclosure provides a combination therapy comprising administering a compound of formula I described herein, and prodrugs, analogs and derivatives thereof, in combination with an immune checkpoint modulator selected from a checkpoint inhibitor, such as an anti-PD-1/PD-L1 antibody, and an agonist of an immune co-stimulatory molecule, such as an anti-OX40 (CD134) agonist antibody. Without being bound by any specific theory, the inventors propose that H1b-ADP and its derivatives described herein may promote the antigen-presenting functions of tumor infiltrating antigen presenting cells (APC) and tumor-specific T cell proliferation and differentiation. In addition, these molecules may also heighten the recruitment of tumor-specific CD8⁺ T cells to tumors by increasing PD-L1 expression in tumor cells.

In embodiments, the disclosure provides methods of modulating an immune response in a subject, the methods comprising administering to the subject a composition comprising a compound of formula I described herein, and prodrugs, analogs and derivatives thereof.

In embodiments, the disclosure provides methods of potentiating an immune response to a target antigen in a subject, the methods comprising administering to the subject a composition comprising a compound of formula I described herein, and prodrugs, analogs and derivatives thereof. In embodiments, the target antigen may be an antigen of an infectious agent, such as a bacterial antigen, a viral antigen, or an antigen of a parasite. In embodiments, the antigen is a tumor antigen. In accordance with any of these embodiments, a compound of formula I described herein, and prodrugs, analogs and derivatives thereof, may serve as an adjuvant to a vaccine composition for the treatment or prevention of a disease or disorder caused by an infectious agent, or for the treatment of cancer, or for the treatment of another disease or disorder that may be treated with a vaccine composition, including, for example, Alzheimer's disease. In embodiments, the antigen is selected from amyloid protein in the treatment of Alzheimer's disease. In embodiments, the antigen is selected from glycoprotein 100 (gp100), mucin 1 (MUC1), and melanoma-associated antigen 3 (MAGEA3) in the treatment of cancer. In embodiments, the cancer is selected from breast, ovarian, or prostate cancer. In embodiments, the cancer is HTLV-1 T-lymphotropic leukemia.

In embodiments, the cancer is melanoma and a compound of formula I described herein, and prodrugs, analogs and derivatives thereof, may serve as an adjuvant to treatment with Talimogene laherparepvec (T-VEC), or may be used in a combination therapy regimen with T-VEC.

In embodiments for the treatment or prevention of an infectious disease, a compound of formula I described herein, and prodrugs, analogs and derivatives thereof, may serve as an adjuvant to a vaccine composition for the treatment or prevention of anthrax, caries, Chagas disease, dengue, diphtheria, ehrlichiosis, hepatitis A or B, herpes, seasonal influenza, Japanese encephalitis, leprosy, lyme disease, malaria, measles, mumps, meningococcal disease, including meningitis and septicemia, Onchocerciasis river blindness, pertussis (whooping cough), pneumococcal disease, polio, rabies, rubella, schistosomiasis, severe acute respiratory syndrome (SARS), shingles, smallpox, syphilis, tetanus, tuberculosis, tularemia, tick-borne encephalitis virus, typhoid fever, trypanosomiasis, yellow fever, and visceral leishmaniasis.

In embodiments for the treatment or prevention of an infectious disease, the a compound of formula I described herein, and prodrugs, analogs and derivatives thereof, may serve as an adjuvant to a vaccine composition for the treatment or prevention of a disease or disorder caused by adenovirus, Coxsackie B virus, cytomegalovirus, eastern equine encephalitis virus, ebola virus, enterovirus 71, Epstein-Barr virus, Haemophilus influenzae type b (Hib), hepatitis C virus (HCV), herpes virus, human immunodeficiency virus (HIV), human papillomavirus (HPV), hookworm, Marburg virus, norovirus, respiratory syncytial virus (RSV), rotavirus, Salmonella typhi, Staphylococcus aureus, Streptococcus pyogenes, varicella, West Nile virus, Yersinia pestis, and Zika virus.

In accordance with any of the foregoing embodiments, the method may comprise administering a vaccine composition or adjuvant comprising a compound of formula I described herein, and prodrugs, analogs and derivatives thereof.

In embodiments, the disclosure provides methods of treating a disease or disorder amendable to treatment by activation of NFkB, p38, and JNK cell signaling pathways in cells of a subject, the method comprising administering to the subject a compound of formula I described herein, and prodrugs, analogs and derivatives thereof. In embodiments, the disease or disorder is caused by a bacterial, viral, or parasitic infection, as described in more detail below, and including for example diseases and disorders caused by the hepatitis C virus (HCV), the hepatitis B virus (HBV), and the human immunodeficiency virus (HIV). In embodiments, the disease or disorder is selected from tuberculosis, meningitis, pneumonia, ulcer, and sepsis. In embodiments, the disease or disorder is selected from rhinitis, asthma, allergy, COPD, inflammatory bowel disease, arthritis, obesity, radiation-induced inflammation, psoriasis, atopic dermatitis, non-alcoholic steatohepatitis (NASH), Alzheimer's disease, systemic lupus, erythematosus (SLE), autoimmune thyroiditis (Grave's disease), multiple sclerosis, ankylosing spondylitis and bullous diseases. In embodiments, the disease or disorder is selected from actinic keratoses, ulcerative colitis, Crohn's disease, and alopecia areata.

In embodiments, the disclosure provides methods of treating or preventing a bacterial, viral, or parasitic infection in a subject in need thereof, the methods comprising administering to the subject a composition comprising a compound of formula I described herein, and prodrugs, analogs and derivatives thereof.

In embodiments, the method is a method of treating or preventing a bacterial infection. In embodiments, the bacterial infection is caused by a Gram-negative or a Gram-positive bacteria. In embodiments, the bacteria is a Gram-negative bacteria selected from the group consisting of Acinetobacter baumanii, Aggregatobacter actinoinycetemcomitans, Bartonella bacilliformis, Bartonella henselae. Bartonella quintana, Bifidobacterium Borrelia, Bortadella pertussis, Brucella sp, Burkholderia cepacis, Burkholderia pseudomallei, Campylobacter jejuni, Cardiobacterium hominis, Campylobacter fetus, Chlamydia pneumonia, Chlymydia trachomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis. Pseudomonas putida, Pseudomonas fluorescens, Pseudomonas acidovorans, Rickettsiae, Salmonella enterica, Salmonella typhi, Salmonella paratyphi types A. B. typhus, Salmonella dublin, Sanionella arizonae, Sahmonella choleraesuis, Serratia marcescens, Schigella dysemneriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus and Yersinia pestitis.

In embodiments, the bacteria is a Gram-positive bacteria selected from the group consisting of Actinomycetes, Bacillus anthracis, Bacillus subtilis, Clostridium tetani, Clostridium perfingens, Clostridium botulinum, Clostridium tetani, Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, and Streptococcus mutants.

In embodiments, the method is a method of treating or preventing a viral infection. In embodiments, the viral infection is caused by a virus selected from the group consisting of Adeno-associated virus, Aichi virus, Alpha virus, Arena virus, Arobovirus, Australian bat lyssavirus, BK polyomavirus, Banna virus, Birnavirus, Bornavirus, bunyamwera virus, Bunyavirus La Crosse, Bunyavirus snowshoe hare, Valicivirus, Cercopithecine herpesvirus, Chandipura virus, Chikugunya virus, Cosavirus A, Coxpox virus, Coxsakievirus, Crimean-Congo hemorrhagic fever virus, Dengue virus, Dhori virus, Dugbe virus, Devenhage virus, Eastern equine encephalitis virus, Ebolavirus, Echovirus, Encephalomyocarditis virus, Epstein-Barr virus, European bat lyssavirus, Flavivirus, GB virus/Hepatitis G virus, Hantaan virus, Hendra virus, hepadnavirus, Hepatitis A virus, Hepatitis B virus, Hepatitis C virus, Hepatitis E virus, Hepatitis delta virus, Herpes simplex virus, horsepox virus, human adenovirus, human astrovirus, human coronavirus, human cytomegalovirus, human enterovirus 68,70, human herpesvirus 1, human herpesvirus 2, human herpesvirus 6, human herpesvirus 7, human herpesvirus 8, human immunodeficiency virus (HIV), human papillomavirus (HPV-6, HPV-11), human spumaretrovirus, human T-lymphotropic virus, human torovirus, Infleunza A virus, Infleunza B virus, Infleunza C virus, Isfaha virus, JC polyomavirus, Japanese encephalitis virus, Junin arenavirus, Kaposi's sarcoma (HHV-8), KI polyomavirus, Kunjin virus, Lagos bat virus, Lake Vitoria marbugvirus, Langat virus, Lassa virus, LMC virus, Lordsdale virus, Louping ill virus, Lymphocytic choriomeningitis virus, Machupovirus, Marmath forest virus, Mayaro virus, MERS coronavirus, Measles virus, Mengo encephalomycarditis virus, Merkel cell polyomavirus, mlluscum contagiosum, parvovirus B19, Mokola virus, Mumps virus, Murray valley encephalitis virus, New York virus, Nipha virus, Norwalk virus, O'nyong-hyong virus, Orf virus, Oropouche virus, Orthomyxovirus, parainfluenza virus, paramyxovaris, parvovirus, Phchinde virus, picomavirus, poliovirus, polyomavirus, poxvirus, Punta toro phleboviris, Puumala virus, rabdovirus, Rabies virus, reovirus, rhinovirus, respiratory syncytial virus, Rift valley fever virus, Rosavirus A, Ross river virus, Rotavirus A, Rotavirus B, Rotavirus C, Rubella virus, Sagiyama virus, Salivirus A, Sandfly fever sicillian virus, Sapporo virus, Semliki forest virus, Seoul virus, Simian foamy virus, Simian virus 5, Sindbis virus, Southampton virus, St. louis encephalitis virus, Tick-borne powassan virus, togavirus, Torque virus, Toscana virus, Uukuniemi virus, Vaccina virus, Varicella-zoster virus, Variola virus, Venezuelan equine encephalitis virus, Vesicular stomatitits virus, Western equine encephalitis virus, UU polyomavirus, West Nile virus, Yaba monkey tumor virus, Yaba-like disease virus, Yellow fever virus, and Zika virus.

In embodiments, the method is a method of treating or preventing a parasitic infection. In embodiments, the parasitic infection is caused by parasite selected from the group consisting of Acanthamoeba spp, American tryppanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

In embodiments, the disclosure provides methods of treating cancer in a subject, the methods comprising administering to the subject a composition comprising a compound of formula I described herein, and prodrugs, analogs and derivatives thereof. In embodiments, the cancer is selected from soft tissue sarcoma, breast cancer, head and neck cancer, melanoma, cervical cancer, bladder cancer, hematologic malignancy, glioblastoma, pancreatic cancer, prostate cancer, colon cancer, breast cancer, renal cancer, lung cancer, merkel cell carcinoma, small intestine cancer, thyroid cancer, acute myelogenous leukemia (AML), acute lymphocytic leukemia (ALL), chronic lymphocytic leukemia (CLL), chronic myelogenous leukemia (CML), gastric cancer, gastrointestinal stromal tumors, non-Hodgkins lymphoma, Hodgkins lymphoma, liver cancer, leukemia, lymphoma, T-cell lymphoma.

In embodiments of any of the methods described here the compound of formula I described herein, and prodrugs, analogs and derivatives thereof, may be administered in combination with one or more additional therapeutic agents or immune modulators, including for example in combination with a vaccine or vaccine adjuvant. In embodiments, the one or more additional therapeutic agents is an inhibitor or antagonist of, or a vaccine against, an immune checkpoint molecule including, for example, the programed cell death 1 (PD-1) receptor (CD279), a ligand of PD-1 (e.g., PD-L1), cytotoxic T-lymphocyte associated protein 4 (CTLA4), tumor necrosis factor receptor superfamily member 9 (alternatively TNFRSF9, 4-1BB) and 4-1BB ligands, tumor necrosis factor receptor superfamily member 4 (alternatively TNFRSF4, OX40) and OX40 ligands, glucocorticoid-induced TNFR-related protein (GITR), Tumor Necrosis Factor Receptor Superfamily Member 7 (alternatively TNFRSF7, cluster of differentiation 27. CD27). TNFRSF25 and TNF-like ligand 1A (TL1A), TNF Receptor Superfamily Member 5 (alternatively TNFRSF5, CD40) and CD40 ligand. Herpesvirus entry mediator (HVEM)-tumor necrosis factor ligand superfamily member 14 (alternatively TNFSF14. LIGHT)-lymphotoxin alpha (LTA), herpesvirus entry mediator-(HVEM)-B- and T-lymphocyte attenuator (BTLA)-CD160 (alternatively TNFSF14), lymphocyte activating gene 3 (LAG3), T-cell immunoglobulin and mucin-domain containing-3 (TIM3), sialic-acid-binding immunoglobulin-like lectins (SIGLECs), inducible T-cell costimulator (ICOS) and ICOS ligand, B7-H3 (B7 family, alternatively CD276), V-set domain-containing T-cell activation inhibitor 1 (VTCN1, alternatively B7-H4), V-Type immunoglobulin domain-containing suppressor of T-cell activation (VISTA), human endogenous retrovirus-H1 long terminal repeat-associating protein 2 (HHLA2)-transmembrane and Immunoglobulin domain containing 2 (TMIGD2), butyrophilins, natural killer cell receptor 2B4 (alternatively NKR2B4, CD244) and B-Cell Membrane Protein (CD48). T-Cell Immunoreceptor with Immunoglobulin (Ig) and immunoreceptor tyrosine-based inhibition motif domains (TIGIT) and Poliovirus receptor (PVR) family members, killer-cell immunoglobulin-like receptors (KIRs), Immunoglobulin-like transcripts (ILTs) and leukocyte immunoglobulin-like receptor (LIRs), natural killer group protein 2 member D (NKG2D) and natural killer group protein 2 member A (NKG2A), major histocompatibility complex (MHC) class I polypeptide-related sequence A (MICA) and MHC class I polypeptide-related sequence B (MICB), natural killer cell receptor 2B4 (CD244), colony stimulating factor 1 receptor (CSF1R), indoleamine 2,3-dioxygenase (IDO), transforming growth factor beta (TGFβ), Adenosine-ecto-nucleotidase triphosphate diphosphohydrolase 1 (CD39)-5′-nucleotidase (CD73), C-X-C motif chemokine receptor 4 (CXCR4) and C-X-C motif chemokine ligand 12 (CXCL12), phosphatidylserine, signal regulatory protein alpha (SIRPA) and integrin associated protein (CD47), vascular endothelial growth factor (VEGF), and neuropilin.

In embodiments of any of the methods described here the compound of formula I described herein, and prodrugs, analogs and derivatives thereof, may be administered in combination with a checkpoint inhibitor or an agonist of an immune co-stimulatory molecule, such as an anti-OX40 (CD134) agonist antibody. In embodiments, the checkpoint inhibitor is a PD-1/PD-L1 inhibitor, such as an anti-PD1 antibody or an anti-PD-L1 antibody, and the ALPK1 agonist is selected from H1b-ADP-6L and H1b-ADP, and prodrugs, analogs and derivatives thereof.

In embodiments, a compound of formula I described herein, and prodrugs, analogs and derivatives thereof, may be administered in combination with one or more immune modulators. In embodiments, the immune modulator may be a vaccine. In embodiments, the vaccine is a vaccine against an infectious agent, as described above. In embodiments, the vaccine is a cancer vaccine. In embodiments, the cancer vaccine targets a tumor antigen selected from glycoprotein 100 (gp100), mucin 1 (MUC1), and melanoma-associated antigen 3 (MAGEA3).

In embodiments, the one or more immune modulators may be a recombinant protein, for example, granulocyte-macrophage colon-stimulating factor (GM-CSF), interleukin 7 (IL-7), IL-12, IL-15, IL-18, or IL-21.

In embodiments of the treatment of cancer, a compound of formula I described herein, and prodrugs, analogs and derivatives thereof, may be administered in combination with a T cell therapy, such as chimeric antigen receptor (CAR) T cell therapy,

In embodiments of the methods for treating cancer a compound of formula I described herein, and prodrugs, analogs and derivatives thereof, may be administered in combination with a PD-1/PD-L1 inhibitor or an agonist of an immune co-stimulatory molecule, such as an anti-OX40 (CD134) agonist antibody. In embodiments, the cancer is selected from advanced melanoma, non-small cell lung cancer, renal cell carcinoma, bladder cancer, liver cancer, gastric cancer, colon cancer, breast cancer, non-Hodgkin's lymphoma, prostate cancer, head and neck cancer, thyroid cancer, brain cancer, acute myeloid leukemia (AML), merkel cell carcinoma, multiple myeloma, cervical cancer, and sarcoma and the method further comprises administering a PD-1/PD-L1 inhibitor or an agonist of an immune co-stimulatory molecule to the subject.

In embodiments of the methods for modulating an immune response or for treating or preventing a bacterial, viral, or parasitic infection, the one or more additional therapeutic agents may be an immune modulator, for example, an inhibitor or antagonist of immune checkpoint molecule. Such molecules generally act as key regulators of the immune system, for example, as co-stimulators of the immune response.

In embodiments, the disclosure also provides a vaccine composition or vaccine adjuvant comprising a compound of formula I described herein, and prodrugs, analogs and derivatives thereof. A vaccine composition described here may further comprise one or more adjuvants.

In embodiments, the disclosure also provides a pharmaceutical composition comprising a compound of formula I described herein, and prodrugs, analogs and derivatives thereof.

In the context of the methods described here, the term “treating” may refer to the amelioration or stabilization of one or more symptoms associated with the disease, disorder or condition being treated. The term “treating” may also encompass the management of disease, disorder or condition, referring to the beneficial effects that a subject derives from a therapy but which does not result in a cure of the underlying disease, disorder, or condition. In the context of the present disclosure, the term “prevention” refers to preventing the recurrence, development, progression or onset of one or more symptoms of the disease, disorder, or condition.

In embodiments where a therapeutically effective amount of a compound or composition is administered to a subject, the therapeutically effective amount is the amount sufficient to achieve a desired therapeutic outcome, for example the amelioration or stabilization of one or more symptoms of the disease, disorder or condition being treated, or in the context of prevention, the amount sufficient to achieve prevention of the recurrence, development, progression or onset of one or more symptoms of the disease, disorder, or condition.

In embodiments, a therapeutically effective amount is the amount required to achieve at least an equivalent therapeutic effect compared to a standard therapy. An example of a standard therapy is an FDA-approved drug indicated for treating the same disease, disorder or condition.

In the context of any of the methods described here, the subject is preferably a human but may be a non-human vertebrate. In other embodiments, the non-human vertebrate may be, for example, a dog, cat, a rodent (e.g., a mouse, a rat, a rabbit), a horse, a cow, a sheep, a goat, a chicken, a duck, or any other non-human vertebrate.

In embodiments, the human subject is selected from an adult human, a pediatric human, or a geriatric human, as those terms are understood by the medical practitioner, for example as defined by the U.S. Food and Drug Administration.

In embodiments, the disclosure provides a composition comprising an ALPK1 agonist, or a composition comprising a polynucleotide encoding ALPK1, or a composition comprising ALPK1 protein, and one or more excipients or carriers, preferably pharmaceutically acceptable excipients or carriers. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Excipients for preparing a pharmaceutical composition are generally those that are known to be safe and non-toxic when administered to a human or animal body. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, and suitable mixtures of any of the foregoing. The particular excipients utilized in a composition will depend upon various factors, including chemical stability and solubility of the compound being formulated and the intended route of administration.

A pharmaceutical composition can be provided in bulk or unit dosage form. It is especially advantageous to formulate pharmaceutical compositions in unit dosage form for ease of administration and uniformity of dosage. The term “unit dosage form” refers to physically discrete units suited as unitary dosages for the subject to be treated; each unit containing a predetermined quantity of an active compound calculated to produce the desired therapeutic effect in association with the required pharmaceutical carrier. A unit dosage form can be an ampoule, a vial, a suppository, a dragee, a tablet, a capsule, an IV bag, or a single pump on an aerosol inhaler.

In therapeutic applications, dose may vary depending on the chemical and physical properties of the active compound as well as clinical characteristics of the subject, including e.g., age, weight, and co-morbidities. Generally, the dose should be a therapeutically effective amount. An effective amount of a pharmaceutical composition is that which provides an objectively identifiable improvement as noted by the clinician or other qualified observer. For example, alleviating a symptom of a disorder, disease or condition.

A pharmaceutical compositions may take any suitable form (e.g. liquids, aerosols, solutions, inhalants, mists, sprays; or solids, powders, ointments, pastes, creams, lotions, gels, patches and the like) for administration by any desired route (e.g. pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). In embodiments, the pharmaceutical composition is in the form of an orally acceptable dosage form including, but not limited to, capsules, tablets, buccal forms, troches, lozenges, and oral liquids in the form of emulsions, aqueous suspensions, dispersions or solutions. Capsules may contain excipients such as inert fillers and/or diluents including starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added.

In embodiments, the pharmaceutical composition is in the form of a tablet. The tablet can comprise a unit dose of a compound described here together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. butylated hydroxytoluene), buffering agents (e.g. phosphate or citrate buffers), and effervescent agents such as citrate/bicarbonate mixtures. The tablet may be a coated tablet. The coating can be a protective film coating (e.g. a wax or varnish) or a coating designed to control the release of the active compound, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecyl sulfate, magnesium aluminum silicate, and triethanolamine.

In embodiments, the pharmaceutical composition is in the form of a hard or soft gelatin capsule. In accordance with this formulation, the compound of the present invention may be in a solid, semi-solid, or liquid form.

In embodiments, the pharmaceutical composition is in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

In embodiments, the pharmaceutical composition is in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions can be prepared in water with the aid of co-solvent or a surfactant. Examples of suitable surfactants include polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides. Dispersions can also be prepared, for example, in glycerol, liquid polyethylene glycols and mixtures of the same in oils.

In embodiments, a compound or composition described here may be administered as monotherapy or adjunctive therapy. In embodiments, a compound or composition described here may be administered alone or in combination with one or more additional therapeutic agents (i.e., additional APIs) or therapies, for example as part of a therapeutic regimen that includes, e.g., aspects of diet and exercise). In embodiments, the methods described here include administration of a compound of formula I described herein, and prodrugs, analogs and derivatives thereof, as the primary therapy. In other embodiments, the administration of a compound of formula I described herein, and prodrugs, analogs and derivatives thereof, is an adjuvant therapy. In either case, the methods of the invention contemplate the administration of a compound of formula I described herein, and prodrugs, analogs and derivatives thereof, in combination with one or more additional therapeutic agents and/or therapies for the treatment or prevention of a disease, disorder, or condition as described here. The terms “therapy” and “therapies” refer to any method, protocol and/or agent that can be used in the prevention, treatment, management or amelioration of a disease, disorder, or condition, one or more symptoms thereof.

The present disclosure also provides packaging and kits comprising pharmaceutical compositions for use in the methods described here. The kit can comprise one or more containers selected from the group consisting of a bottle, a vial, an ampoule, a blister pack, and a syringe. The kit can further include one or more of instructions for use, one or more syringes, one or more applicators, or a sterile solution suitable for reconstituting a compound or composition described here.

### Preparation of Compounds of Formula I and Exemplary Compounds

The compounds of formula I in which L¹ is O (compound VI) can be made by general synthetic method as illustrated in Scheme 1. Compound II (“PG” refers to a protection group) can be obtained by compound I (when M is OH) with protected phosphorochloridate under basic condition or appropriate protected phosphate under Mitsunobu reaction condition. Compound II can be obtained as a mixture of alpha and beta isomers which can be separated on silica gel chromatography. The beta isomer of compound II is deprotected under 1-4 atm of H₂ catalyzed by Pd/C or PtO₂ to give compound III. Coupling of compound IV with morpholine, or another suitable base, by DCC in an appropriate solution such as t-BuOH/H₂O to give compound V. Coupling of compound III and compound V in an appropriate solvent such as pyridine with an appropriate catalyst such as tetrazole under room temperature for 24-72 h provides compound VI.

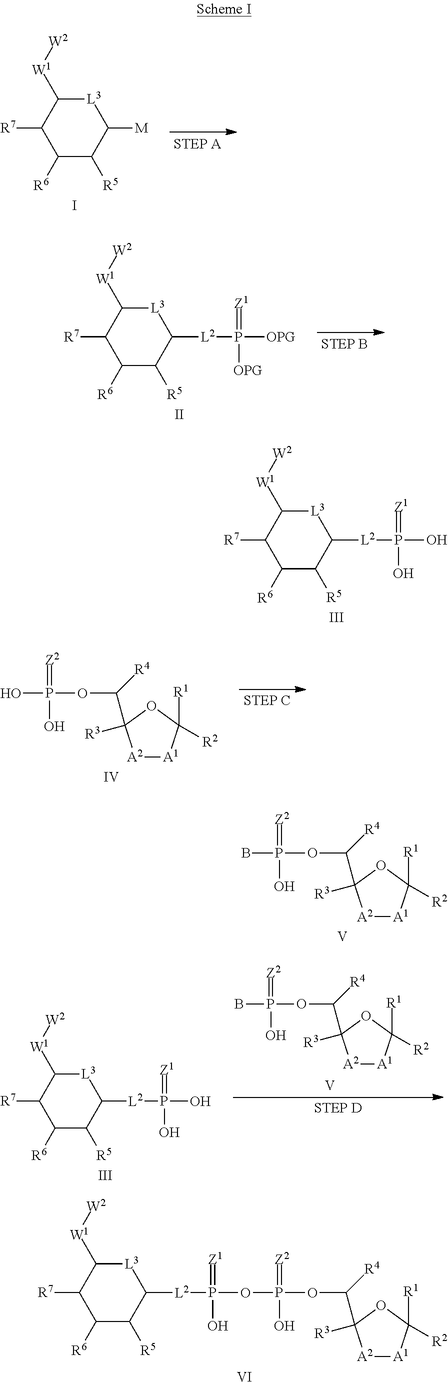

The compounds of formula I in which Z² is S (L¹ is O, compound XI), can be synthesized as illustrated in Scheme II. Compound VIII (“PG” refers to a protection group) can be obtained by reaction of compound VII and protected dialkylphosphoramidite in a suitable solvent like dichloromethane and at temperature ranging from −10 to 25° C. Coupling of VIII and III can be accomplished in a suitable solvent like DMF under inert gas system at temperature below 25° C. to give compound IX, which is oxidized by sulfur in situ to provide compound X. De-preotection of compound X gives the final compound XI.

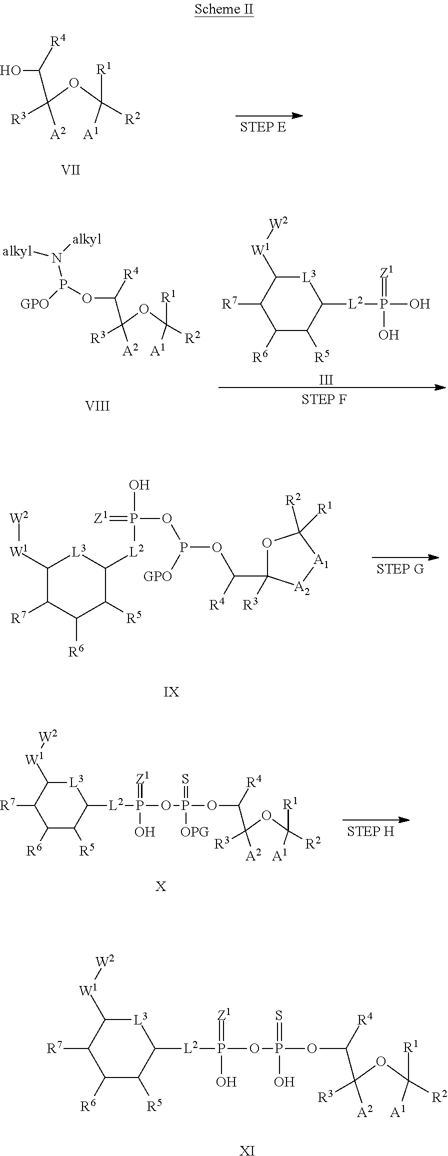

The compounds of formula I in which Z² is S (L¹ is O, compound XV) can be synthesized by the alternatively method as illustrated in Scheme III. The compound III can be activated by forming imidazole salt under 10 to 40° C. in a suitable solvent like DMF under inert gas system. Compound VII is introduced phosphate by reaction with phenoxyphosphonoyloxybenzene. Following the oxidation with sulfur under 0-10° C., compound XIV can be obtained. The coupling of compound XII and XIV under mild condition, like 0-40° C. in a suitable solvent like DMF inert gas system with the catalyst of Lewis acid provides the final compound XV.

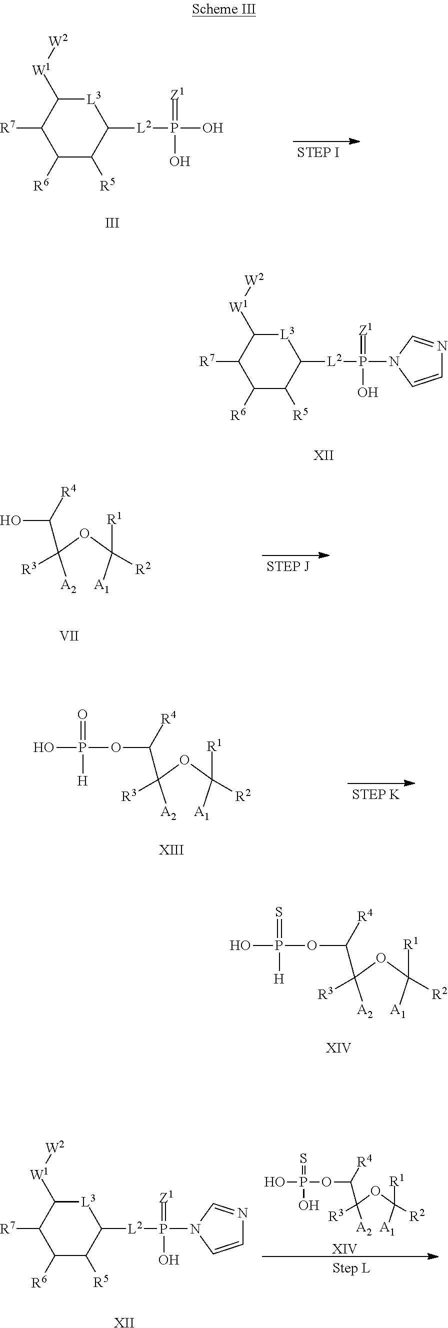

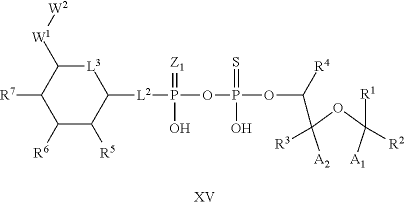

The compounds of formula I in which L¹ is CH₂ (compound XVIII) can be made by the general synthetic method as illustrated in Scheme IV. Mitsunobu reaction of compound I (when M is OH) and the protected methyl diphosphate at 30-50° C. for 2-4 h provides compound XVI (“PG” refers to a protection group). Compound XVI undergoes a second Mitsunobu reaction with compound VI under the similar condition gives compound XVII. Deprotection reaction of compound XVII provides the final compound XVIII.

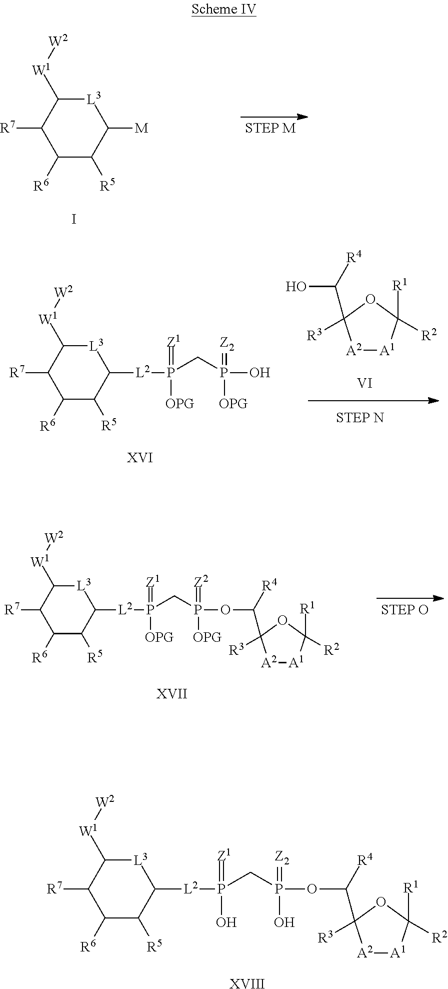

The compounds of formula I in which L¹ is CF₂ (compound XXV) can be made by general synthetic method as illustrated in Scheme V. Compound XIX (“PG” refers to a protection group) is converted to the protected di-fluoromethyl diphosphate compound XX by using N-fluorobenzenesulfonimide (NFSI) under basic NaH conditions in a suitable solvent starting from a low reaction temperature of −20° C. to 0° C. Selective removal of one of the protection groups in compound XX yields compound XXI. Compound VII is converted to compound XXII by converting the hydroxyl group into a leaving group, such as OTs, OMs or halogen. The Mitsunobu reaction of compound I (when M is OH) and compound XXI at 30-50° C. for 2-4 h yields compound XXIII. Deprotection of compound XXIII yields compound XXIV. The coupling of compound XXIV with compound XXII in a suitable solvent such as CH₃CN with the base Bu₄N yields the final compound XXV.

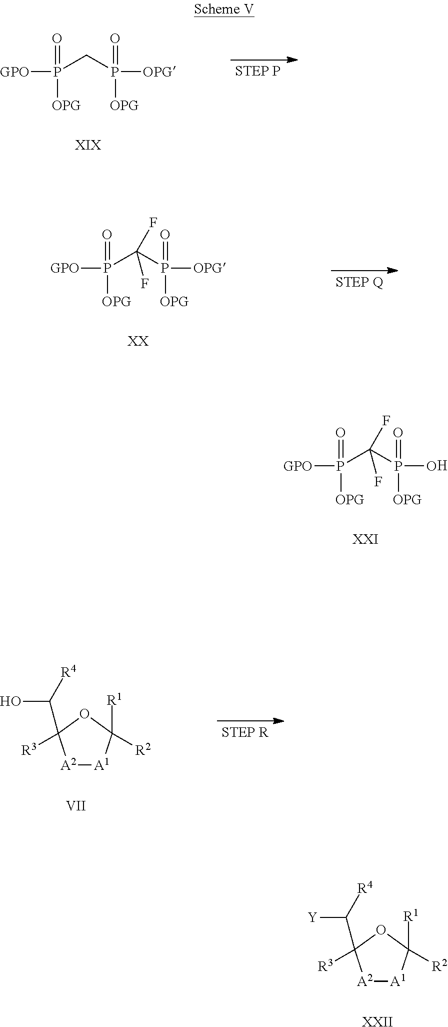

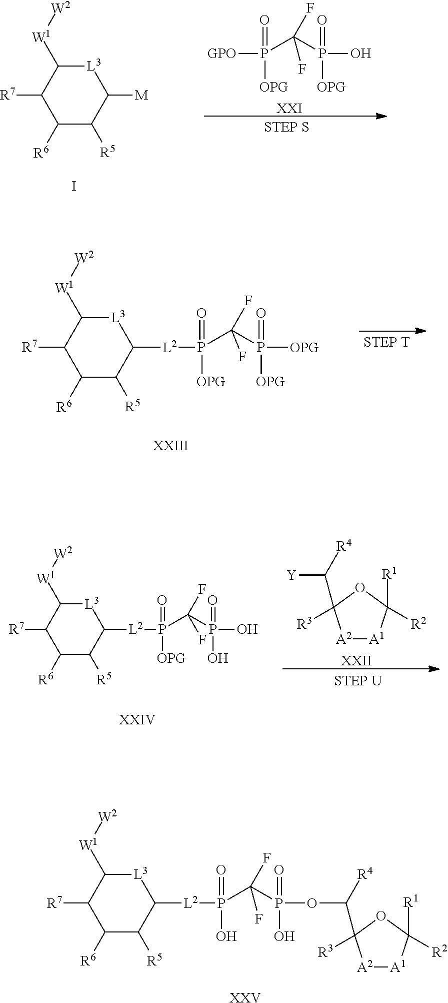

The compounds of formula IC in which A¹ is S (compound XXXII) can be made by a general synthetic method as illustrated in Scheme VI. Reaction of compound XXVI with protected 2-hydroxyacetaldehyde similar to 2-oxoethyl benzoate in a suitable solvent yields compound XXVII (“PG” refers to a protection group) as a mixture of two isomers. Alternatively, the reaction of compound XXVI with 2-oxoethyl benzoate in the presence of an organic base (e.g., triethylamine), phenyl acetate, surfactant-treated subtilisin (STS), and Carlsberg in a suitable solvent such as THF yields compound XXVII in the R configuration. Using CAL B instead of STS could yield compound XXVII in the S configuration (Reference: Hu, L et al. Chem. Commun., 2013, 49, 10376-10378). Compound XXVII reacted with SnCl₄ in a suitable solvent solution yields compound XXVIII. Deprotection of compound XXVIII yields compound XXIX. Phosphorination of compound XXIX can be accomplished by reacting it with POCl₃ and pyridine in a suitable solvent to yield compound XXX. The remaining reactions of converting compound XXX to the final compound XXXII can be done by the same reaction procedures as described in Scheme I.

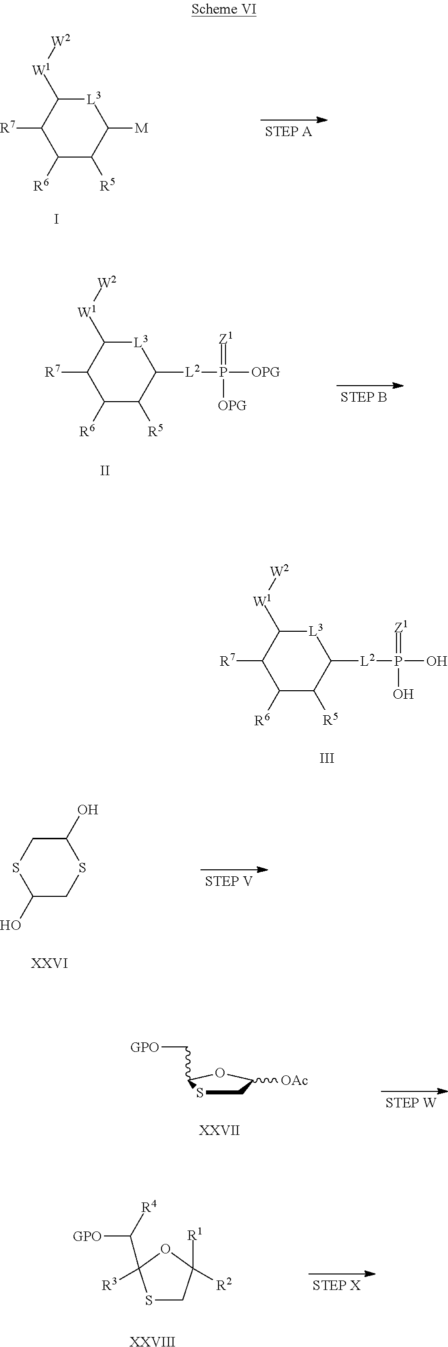

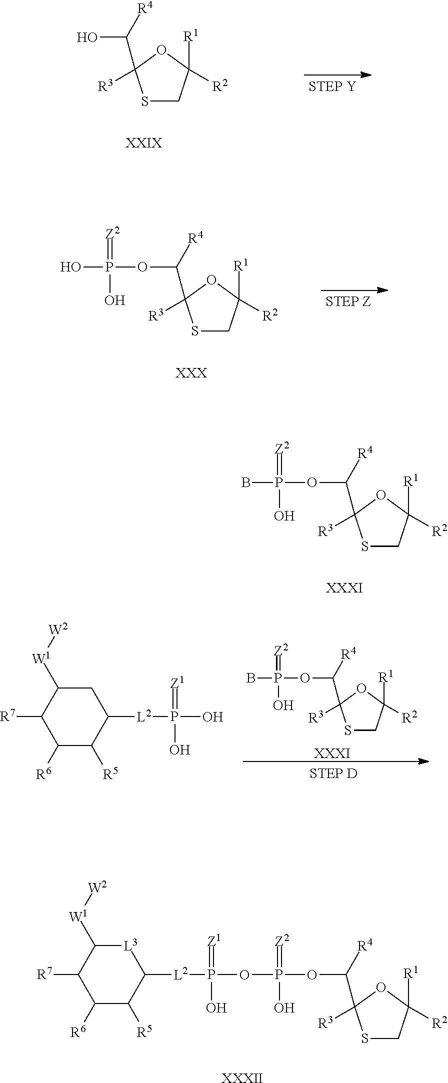

The compounds of formula I in which L¹ is O (compound XXXVII) can be made by general synthetic method as illustrated in Scheme VII. Compound XXXIV (when M is OH) is obtained by the condensation of compound XXXIII with corresponding acid under condensation reagent like EDCI. Compound XXXV (“PG” refers to a protection group) can be obtained by compound XXXIV with protected phosphorochloridate under basic condition or appropriate protected phosphate under Mitsunobu reaction condition. Compound XXXV can be obtained as a mixture of alpha and beta isomers which can be separated on silica gel chromatography. The beta isomer of compound XXXV is deprotected under 1-4 atm of H₂ catalyzed by Pd/C or PtO₂ to give compound XXXVI. Coupling of compound IV with morpholine, or another suitable base, by DCC in an appropriate solution such as t-BuOH/H₂O to give compound V. Coupling of compound V and compound XXXVII in an appropriate solvent such as pyridine with an appropriate catalyst such as tetrazole under room temperature for 24-72 h provides compound XXXVIII.

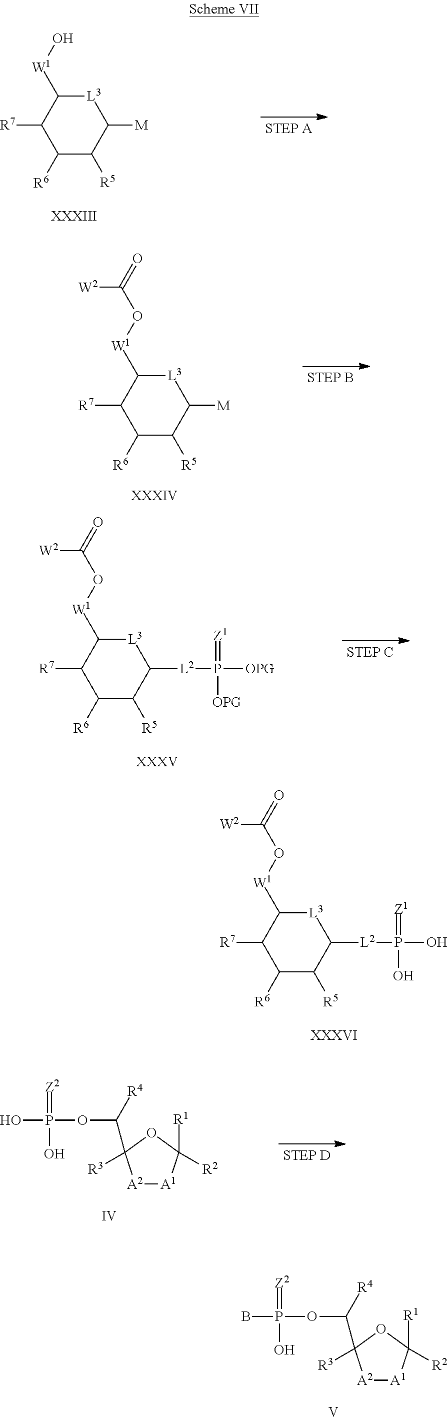

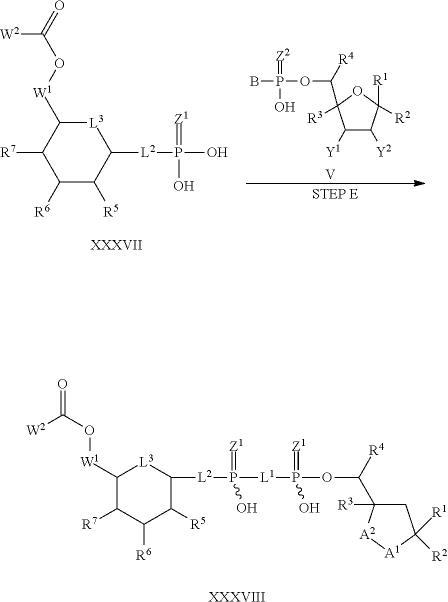

Table 1 lists exemplary compounds prepared according to the procedures as described herein.

TABLE 1
Exemplary Compounds

| | |
|---|---|
| 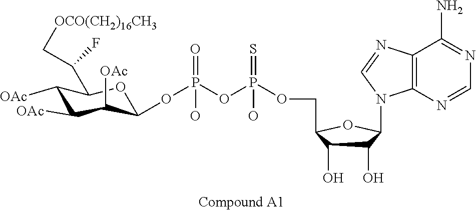 | (2S,3S,4S,5S,6S)-2-(((((((2R,3S,4R,5R)-5- (6-amino-9H-purin-9-yl)-3,4- dihydroxytetrahydrofuran-2- yl)methoxy)(hydroxy)phosphorothioyl)oxy) (hydroxy)phosphoryl)oxy)-6-((S)-1-fluoro- 2-(stearoyloxy)ethyl)tetrahydro-2H-pyran- 3,4,5-triyl triacetate |
| 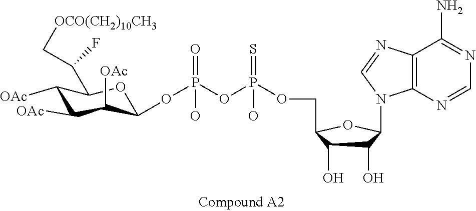 | (2S,3S,4S,5S,6S)-2-(((((((2R,3S,4R,5R)-5- (6-amino-9H-purin-9-yl)-3,4- dihydroxytetrahydrofuran-2- yl)methoxy)(hydroxy)phosphorothioyl)oxy) (hydroxy)phosphoryl)oxy)-6-((S)-2- (dodecanoyloxy)-1-fluoroethyl)tetrahydro- 2H-pyran-3,4,5-triyl triacetate |
| 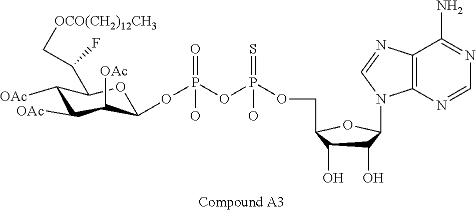 | 2S,3R,4S,5S)-2-(((((((2R,3R,4R,5R)-5-(6- amino-9H-purin-9-yl)-3,4- dihydroxytetrahydrofuran-2- yl)methoxy)(hydroxy)phosphorothioyl)oxy) (hydroxy)phosphoryl)oxy)-6-((S)-1-fluoro- 2-(tetradecanoyloxy)ethyl)tetrahydro-2H- pyran-3,4,5-triyl triacetate |
| 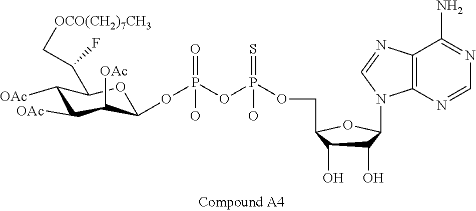 | 2S,3R,4S,5S)-2-(((((((2R,3R,4R,5R)-5-(6- amino-9H-purin-9-yl)-3,4- dihydroxytetrahydrofuran-2- yl)methoxy)(hydroxy)phosphorothioyl)oxy) (hydroxy)phosphoryl)oxy)-6-((S)-1-fluoro- 2-(nonanoyloxy)ethyl)tetrahydro-2H-pyran- 3,4,5-triyl triacetate |
| 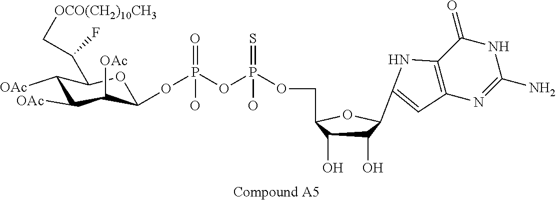 | (2S,3S,4S,5S,6S)-2-(((((((2R,3S,4R,5S)-5- (2-amino-4-oxo-4,5-dihydro-3H- pyrrolo[3,2-d]pyrimidin-6-yl)-3,4- dihydroxytetrahydrofuran-2- yl)methoxy)(hydroxy)phosphorothioyl)oxy) (hydroxy)phosphoryl)oxy)-6-((S)-2- (dodecanoyloxy)-1-fluoroethyl)tetrahydro- 2H-pyran-3,4,5-triyl triacetate |
| 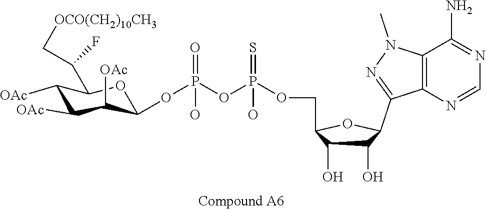 | (2S,3S,4S,5S,6S)-2-(((((((2R,3S,4R,5S)-5- (7-amino-1-methyl-1H-pyrazolo[4,3- d]pyrimidin-3-yl)-3,4- dihydroxytetrahydrofuran-2- yl)methoxy)(hydroxy)phosphorothioyl)oxy) (hydroxy)phosphoryl)oxy)-6-((S)-2- (dodecanoyloxy)-1-fluoroethyl)tetrahydro- 2H-pyran-3,4,5-triyl triacetate |
| 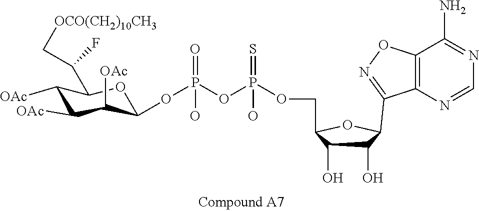 | (2S,3S,4S,5S,6S)-2-(((((((2R,3S,4R,5S)-5- (7-aminoisoxazolo[4,5-d]pyrimidin-3-yl)- 3,4-dihydroxytetrahydrofuran-2- yl)methoxy)(hydroxy)phosphorothioyl)oxy) (hydroxy)phosphoryl)oxy)-6-((S)-2- (dodecanoyloxy)-1-fluoroethyl)tetrahydro- 2H-pyran-3,4,5-triyl triacetate |
| 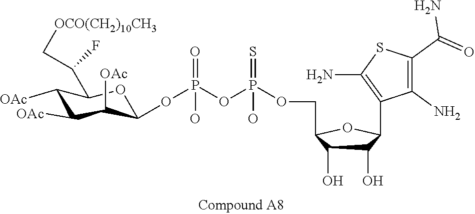 | (2S,3S,4S,5S,6S)-2-(((((((2R,3S,4R,5S)-5- (2,4-diamino-5-carbamoylthiophen-3-yl)- 3,4-dihydroxytetrahydrofuran-2- yl)methoxy)(hydroxy)phosphorothioyl)oxy) (hydroxy)phosphoryl)oxy)-6-((S)-2- (dodecanoyloxy)-1-fluoroethyl)tetrahydro- 2H-pyran-3,4,5-triyl triacetate |
| 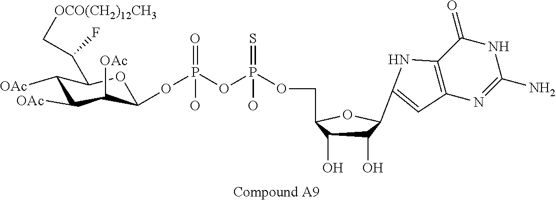 | (2S,3R,4S,5S)-2-(((((((2R,3R,4R,5S)-5-(2- amino-4-oxo-4,5-dihydro-3H-pyrrolo[3,2- d]pyrimidin-6-yl)-3,4- dihydroxytetrahydrofuran-2- yl)methoxy)(hydroxy)phosphorothioyl)oxy) (hydroxy)phosphoryl)oxy)-6-((S)-1-fluoro- 2-(tetradecanoyloxy)ethyl)tetrahydro-2H- pyran-3,4,5-triyl triacetate |
| 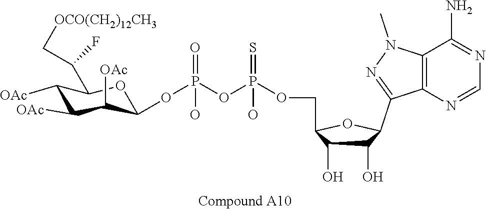 | (2S,3R,4S,5S)-2-(((((((2R,3R,4R,5S)-5-(7- amino-1-methyl-1H-pyrazolo[4,3- d]pyrimidin-3-yl)-3,4- dihydroxytetrahydrofuran-2- yl)methoxy)(hydroxy)phosphorothioyl)oxy) (hydroxy)phosphoryl)oxy)-6-((S)-1-fluoro- 2-(tetradecanoyloxy)ethyl)tetrahydro-2H- pyran-3,4,5-triyl triacetate |
| 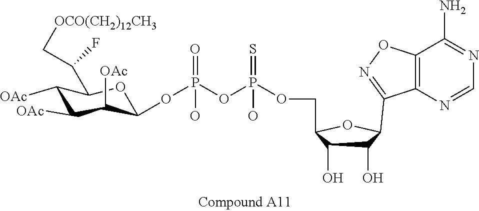 | (2S,3R,4S,5S)-2-(((((((2R,3R,4R,5S)-5-(7- aminoisoxazolo[4,5-d]pyrimidin-3-yl)-3,4- dihydroxytetrahydrofuran-2- yl)methoxy)(hydroxy)phosphorothioyl)oxy) (hydroxy)phosphoryl)oxy)-6-((S)-1-fluoro- 2-(tetradecanoyloxy)ethyl)tetrahydro-2H- pyran-3,4,5-triyl triacetate |
| 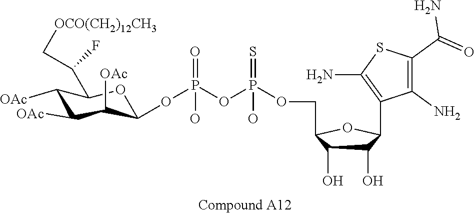 | (2S,3R,4S,5S)-2-(((((((2R,3R,4R,5S)-5- (2,4-diamino-5-carbamoylthiophen-3-yl)- 3,4-dihydroxytetrahydrofuran-2- yl)methoxy)(hydroxy)phosphorothioyl)oxy) (hydroxy)phosphoryl)oxy)-6-((S)-1-fluoro- 2-(tetradecanoyloxy)ethyl)tetrahydro-2H- pyran-3,4,5-triyl triacetate |
| 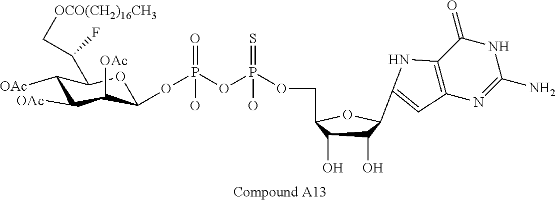 | (2S,3R,4S,5S)-2-(((((((2R,3R,4R,5S)-5-(2- amino-4-oxo-4,5-dihydro-3H-pyrrolo[3,2- d]pyrimidin-6-yl)-3,4- dihydroxytetrahydrofuran-2- yl)methoxy)(hydroxy)phosphorothioyl)oxy) (hydroxy)phosphoryl)oxy)-6-((S)-1-fluoro- 2-(stearoyloxy)ethyl)tetrahydro-2H-pyran- 3,4,5-triyl triacetate |
| 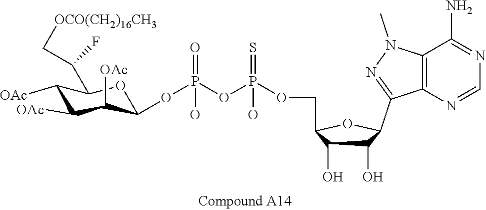 | (2S,3R,4S,5S)-2-(((((((2R,3R,4R,5S)-5-(7- amino-1-methyl-1H-pyrazolo[4,3- d]pyrimidin-3-yl)-3,4- dihydroxytetrahydrofuran-2- yl)methoxy)(hydroxy)phosphorothioyl)oxy) (hydroxy)phosphoryl)oxy)-6-((S)-1-fluoro- 2-(stearoyloxy)ethyl)tetrahydro-2H-pyran- 3,4,5-triyl triacetate |
| 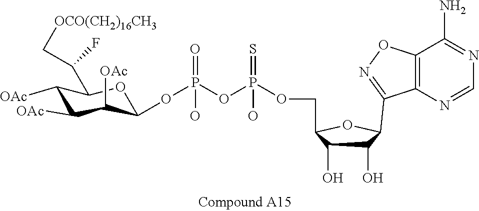 | (2S,3R,4S,5S)-2-(((((((2R,3R,4R,5S)-5-(7- aminoisoxazolo[4,5-d]pyrimidin-3-yl)-3,4- dihydroxytetrahydrofuran-2- yl)methoxy)(hydroxy)phosphorothioyl)oxy) (hydroxy)phosphoryl)oxy)-6-((S)-1-fluoro- 2-(stearoyloxy)ethyl)tetrahydro-2H-pyran- 3,4,5-triyl triacetate |
| 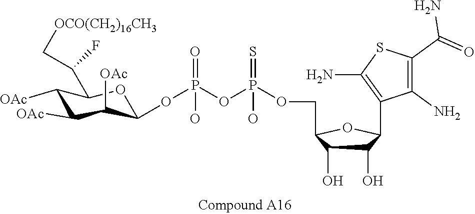 | (2S,3R,4S,5S)-2-(((((((2R,3R,4R,5S)-5- (2,4-diamino-5-carbamoylthiophen-3-yl)- 3,4-dihydroxytetrahydrofuran-2- yl)methoxy)(hydroxy)phosphorothioyl)oxy) (hydroxy)phosphoryl)oxy)-6-((S)-1-fluoro- 2-(stearoyloxy)ethyl)tetrahydro-2H-pyran- 3,4,5-triyl triacetate |
| 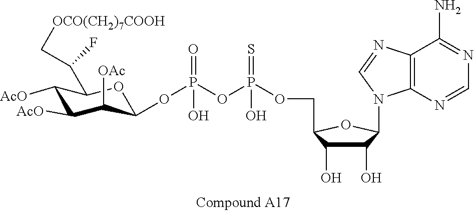 | 9-((2S)-2-fluoro-2-((2S,3S,4S,5S,6S)-3,4,5- triacetoxy-6-(((((((2R,3S,4R,5R)-5-(6- amino-9H-purin-9-yl)-3,4- dihydroxytetrahydrofuran-2- yl)methoxy)(hydroxy)phosphorothioyl)oxy) (hydroxy)phosphoryl)oxy)tetrahydro-2H- pyran-2-yl)ethoxy)-9-oxononanoic acid |
| 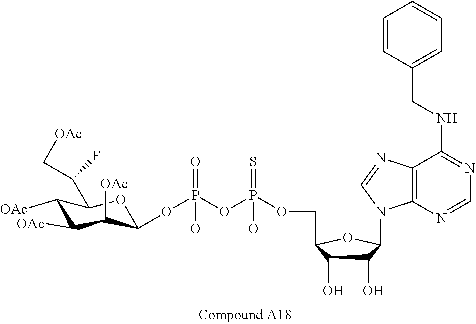 | (2S,3S,4S,5S,6S)-2-((S)-2-acetoxy-1- fluoroethyl)-6-(((((((2R,3S,4R,5R)-5-(6- (benzylamino)-9H-purin-9-yl)-3,4- dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |
| 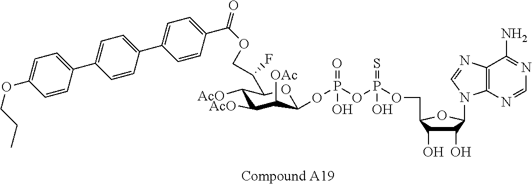 | (2S,3S,4S,5S,6S)-2-(((((((2R,3S,4R,5R)-5- (6-amino-9H-purin-9-yl)-3,4- dihydroxytetrahydrofuran-2- yl)methoxy)(hydroxy)phosphorothioyl)oxy) (hydroxy)phosphoryl)oxy)-6-((S)-1-fluoro- 2-((4″-propoxy-[1,1′:4′,1″-terphenyl]-4- carbonyl)oxy)ethyl)tetrahydro-2H-pyran- 3,4,5-triyl triacetate |
| 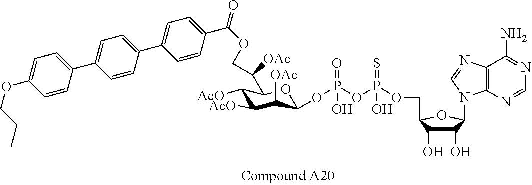 | (2R,3R,4S,5S,6S)-2-((R)-1-acetoxy-2-((4″- propoxy-[1,1′:4′,1″-terphenyl]-4- carbonyl)oxy)ethyl)-6-(((((((2R,3S,4R,5R)- 5-(6-amino-9H-purin-9-yl)-3,4- dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |
| 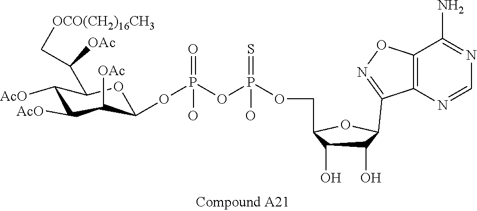 | (2R,3R,4S,5S,6S)-2-((R)-1-acetoxy-2- (stearoyloxy)ethyl)-6-(((((((2R,3S,4R,5S)-5- (7-aminoisoxazolo[4,5-d]pyrimidin-3-yl)- 3,4-dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |
| 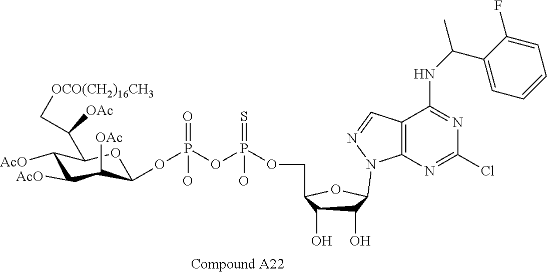 | (2R,3R,4S,5S,6S)-2-((R)-1-acetoxy-2- (stearoyloxy)ethyl)-6-(((((((2R,3S,4R,5R)- 5-(6-chloro-4-((1-(2- fluorophenyl)ethyl)amino)-1H- pyrazolo[3,4-d]pyrimidin-1-yl)-3,4- dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |
| 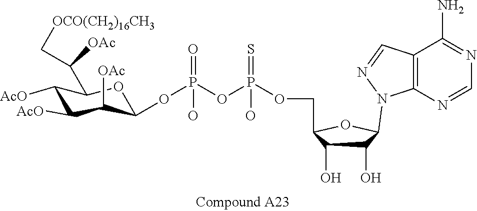 | (2R,3R,4S,5S,6S)-2-((R)-1-acetoxy-2- (stearoyloxy)ethyl)-6-(((((((2R,3S,4R,5R)- 5-(4-amino-1H-pyrazolo[3,4-d]pyrimidin-1- yl)-3,4-dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |
| 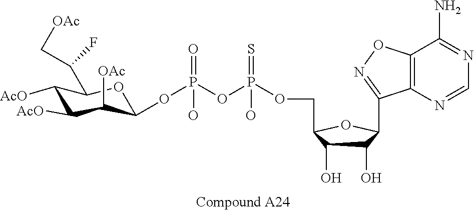 | (2S,3S,4S,5S,6S)-2-((S)-2-acetoxy-1- fluoroethyl)-6-(((((((2R,3S,4R,5S)-5-(7- aminoisoxazolo[4,5-d]pyrimidin-3-yl)-3,4- dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |
| 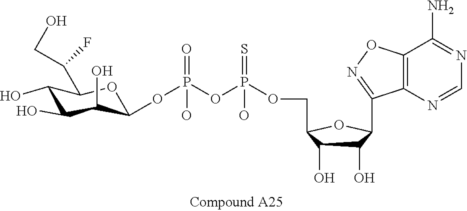 | 5-(7-aminoisoxazolo[4,5-d]pyrimidin-3-yl)-- (D-glycero-β-D-manno-6-fluoro- heptopyranosyl) (hydroxy)phosphorothioyloxyphosphate |
| 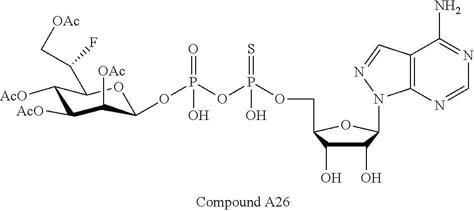 | (2S,3S,4S,5S,6S)-2-((S)-2-acetoxy-1- fluoroethyl)-6-(((((((2R,3S,4R,5R)-5-(4- amino-1H-pyrazolo[3,4-d]pyrimidin-1-yl)- 3,4-dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |
| 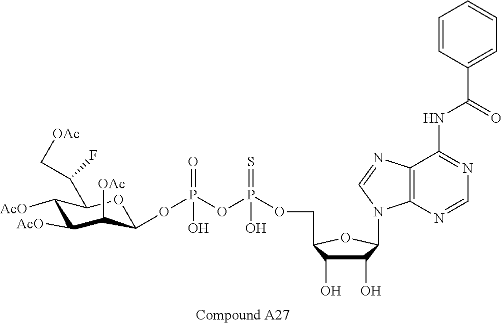 | (2S,3S,4S,5S,6S)-2-((S)-2-acetoxy-1- fluoroethyl)-6-(((((((2R,3S,4R,5R)-5-(6- benzamido-9H-purin-9-yl)-3,4- dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |
| 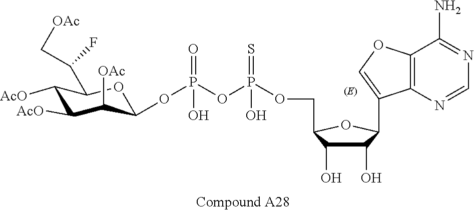 | (2S,3S,4S,5S,6S)-2-((S)-2-acetoxy-1- fluoroethyl)-6-(((((((2R,3S,4R,5S)-5-(4- aminofuro[3,2-d]pyrimidin-7-yl)-3,4- dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |
| 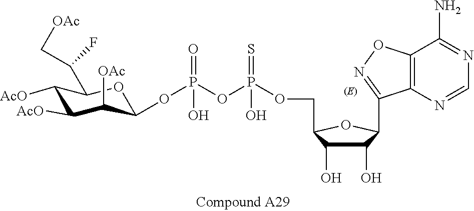 | (2S,3S,4S,5S,6S)-2-((S)-2-acetoxy-1- fluoroethyl)-6-(((((((2R,3S,4R,5S)-5-(7- aminoisoxazolo[4,5-d]pyrimidin-3-yl)-3,4- dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |
| 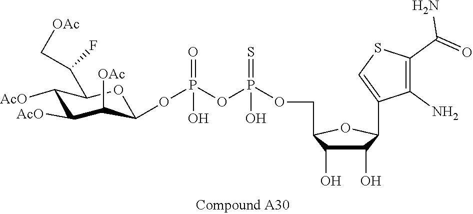 | (2S,3S,4S,5S,6S)-2-((S)-2-acetoxy-1- fluoroethyl)-6-(((((((2R,3S,4R,5S)-5-(4- amino-5-carbamoylthiophen-3-yl)-3,4- dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |
| 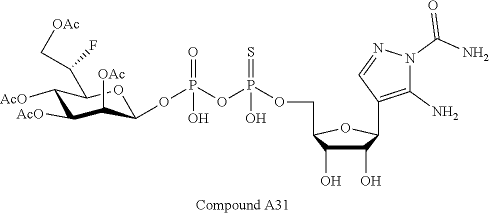 | (2S,3S,4S,5S,6S)-2-((S)-2-acetoxy-1- fluoroethyl)-6-(((((((2R,3S,4R,5S)-5-(5- amino-1H-pyrazol-4-yl)-3,4- dihydroxytetrahydrofuran-2- yl)methoxy)(hydroxy)phosphorothioyl)oxy) (hydroxy)phosphoryl)oxy)tetrahydro-2H- pyran-3,4,5-triyl triacetate |

### Synthesis of Representative Compounds of Formula (I):

All moisture-sensitive reactions were performed using syringe-septum cap techniques under Ar. Analytical thin layer chromatography (TLC) was performed on Silica gel 60 F 254 Plates (Qindao, 0.25 mm thickness). ¹H-NMR spectra were recorded with a Varian-400 spectrometer, and chemical shifts were reported as (ppm) values relative to internal tetramethylsilane or the residual proton of the deuterated solvent. ¹³C-NMR spectra were recorded with a Varian-400 spectrometer, and chemical shifts were reported as δ (ppm) values relative to internal tetramethylsilane or the residual proton of the deuterated solvent. ³¹P-NMR spectra were recorded with a Varian-400 spectrometer, and chemical shifts were reported as δ (ppm) values relative to external 85% phosphoric acid. ¹H-NMR spectra are tabulated as follows: chemical shift, multiplicity (br=broad, s=singlet, d=doublet, t=triplet, q=quartet, m=multiplet), number of protons, and coupling constant(s).

### Compound A1

Step 1. Preparation of compound 1-((2R,3S,4S,5S,6S)-3,4,5-tris(benzyloxy)-6-methoxytetrahydro-2H-pyran-2-yl)-2-(trityloxy)ethan-1-ol.

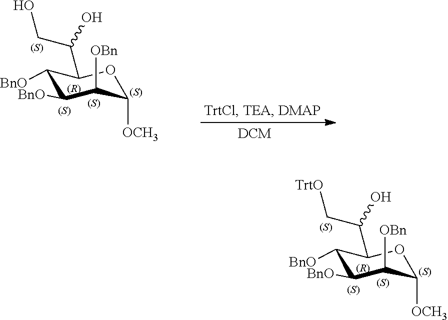

To a solution of compound 1-((2R,3S,4S,5S,6S)-3,4,5-tris(benzyloxy)-6-methoxytetrahydro-2H-pyran-2-yl)ethane-1,2-diol (17.4 g, 35.2 mmol; Tiehai Li et al., (2014) Bioorg. Med. Chem. 22: 1139-1147; Shinsuke Inuki et al., Org. Lett. (2017), 19: 3079-3082), TEA (7.1 g, 70.4 mmol, 9.8 mL) and DMAP (2.2 g, 17.6 mmol) in DCM (200 mL) was added TrtCl (19.6 g, 70.4 mmol). The mixture was stirred at 50° C. for 20 h. The reaction mixture was quenched with H₂O (100 mL) and then separated. The aqueous (aq.) layer was extracted with DCM (60 mL×2). The combined organic layer was washed with brine (150 mL), dried over Na₂SO₄, filtered and concentrated to give a residue. The residue was purified by flash silica gel chromatography (PE:EA=1:0 to 1:1). The desired compound (24.6 g, yield: 95%, 93% purity) was obtained as a pale yellow oil. MS (ESI) m/z (M+H)⁺:782.4.

Step 2. Preparation of compound 1-((2S,3S,4S,5S,6S)-3,4,5-tris(benzyloxy)-6-methoxytetrahydro-2H-pyran-2-yl)-2-(trityloxy)ethan-1-one.

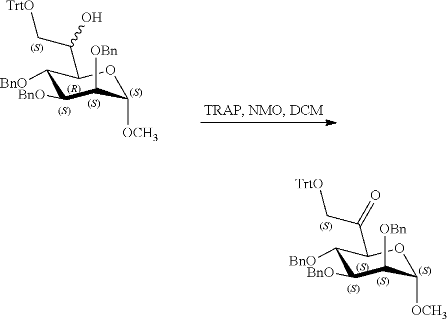

The mixture of product obtained from step 1 above (24.6 g, 33.4 mmol), NMO (19.6 g, 166.9 mmol, 17.6 mL) and 4A molecular sieve (24 g, 33.4 mmol) in DCM (250 mL) was stirred at 25° C. for 0.5 h. Then TPAP (1.17 g, 3.34 mmol) was added at 0° C. The mixture was stirred at 25° C. for 4 h. The mixture was filtered and washed with DCM (50 mL×3). The filtrate was concentrated under vacuum. The residue was purified by flash silica gel chromatography (PE:EA=1:0 to 4:1). The desired product (21.7 g, Yield: 85.6%) was obtained as a light yellow oil.

MS (ESI) m/z (M+H)⁺: 757.3.

¹H NMR (400 MHz, CDCl₃): δ 7.45-7.25 (m, 30H), 4.72-4.52 (m, 6H), 4.20-4.07 (m, 4H), 3.99 (s, 2H), 3.68-3.67 (m, 1H), 3.22 (s, 3H).

Step 3. Preparation of (R)-1-((2R,3S,4S,5S,6S)-3,4,5-tris(benzyloxy)-6-methoxytetrahydro-2H-pyran-2-yl)-2-(trityloxy)ethan-1-ol.

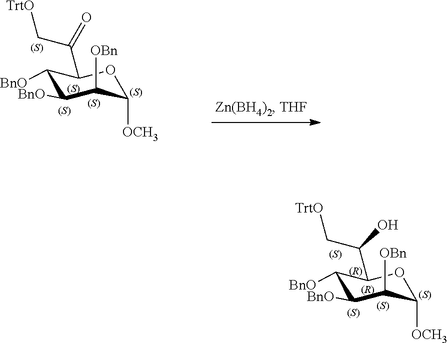

To the solution of the product obtained from step 2 above (21.7 g, 29.5 mmol) in THF (200 mL) was added Zn(BH₄)₂ (0.5 M, 66.7 mL) dropwisely at 0° C. for 0.5 h. The reaction was carefully quenched with H₂O (50 mL). The organic layer was extracted with ethyl acetate (150 mL×3). The organic layer was dried over Na₂SO₄ and concentrated under vacuum. The residue was purified by flash silica gel chromatography (PE:EA=1:0 to 7:1). The desired compound (19.5 g, Yield: 88.27%, 98.5% purity) was obtained as a colorless oil.

MS (ESI) m/z (M+H)⁺: 759.3.

Step 4. Preparation of compound (2S,3S,4S,5S,6S)-3,4,5-tris(benzyloxy)-2-((S)-1-fluoro-2-(trityloxy)ethyl)-6-methoxytetrahydro-2H-pyran.

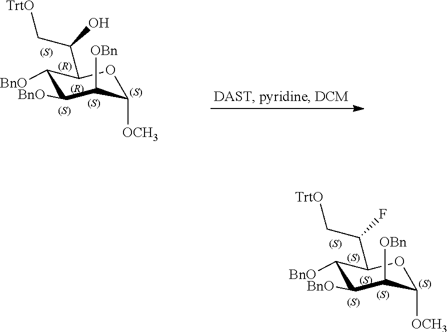

To the mixture of the compound of the product of Step 3 above (9.5 g, 12.9 mmol) in DCM (100 mL) were added DAST (10.4 g, 64.5 mmol, 8.5 mL) and pyridine (10.2 g, 128.9 mmol, 10.4 mL) at 0° C. The mixture was stirred at 25° C. for 16 h. The reaction was quenched with sat. NaHCO₃(100 mL) carefully. The mixture was extracted with DCM (100 mL×3). The combined organic layers were washed with 2N HCl (150 mL), dried over Na₂SO₄ and concentrated under vacuum. The residue was purified by flash silica gel chromatography (PE:EA=1:0 to 12:1). The desired compound (4.2 g, Yield: 44.1%) was obtained as a light yellow oil.

¹H NMR (400 MHz, CDCl₃): δ 7.38-7.18 (m, 30H), 4.92-4.61 (m, 2H), 4.53-4.51 (m, 6H), 4.06-4.02 (m, 1H), 3.77-3.75 (m, 1H), 3.65-3.51 (m, 3H), 3.14-3.06 (m, 1H), 2.96 (s, 3H).

Step 5. Preparation of (S)-2-fluoro-2-((2S,3S,4S,5S,6S)-3,4,5-tris(benzyloxy)-6-methoxytetrahydro-2H-pyran-2-yl)ethyl stearate.

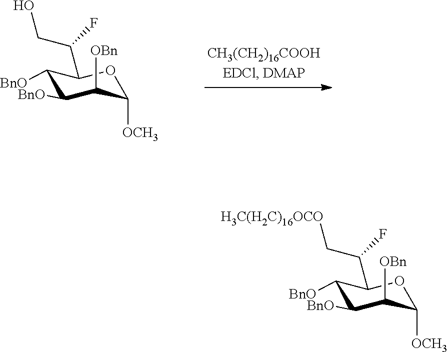

To a solution of (S)-2-fluoro-2-((2S,3S,4S,5S,6S)-3,4,5-tris(benzyloxy)-6-methoxytetrahydro-2H-pyran-2-yl)ethan-1-ol (1.5 g, 3.02 mmol) and stearic acid (2.58 g, 9.06 mmol) in DCM (20 mL) was added EDCI (1.74 g, 9.06 mmol), DMAP (369 mg, 3.02 mmol). The mixture was stirred at 20° C. for 1 h. The solvent was removed in vacuo. The residue was washed with H₂O (20 mL), extracted with EtOAc (30 mL×2). The organics were collected and concentrated. The residue was purified by column (PE:EA=10:1) to give the desired compound (1.49 g, yield: 65%) as colorless oil.

Step 6. Preparation of (S)-2-((2S,3S,4S,5S)-6-acetoxy-3,4,5-tris(benzyloxy)tetrahydro-2H-pyran-2-yl)-2-fluoroethyl stearate.

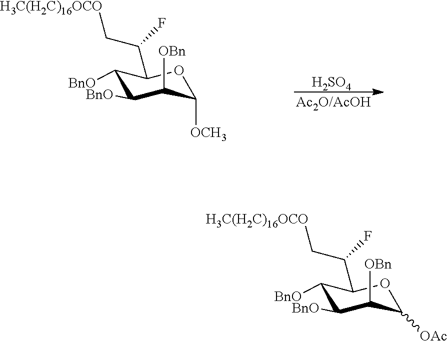

To a solution of compound obtained from step 5 above (1.49 g, 1.95 mmol) in AcOH (4.5 mL) and Ac₂O (4.5 mL) was added H₂SO₄ (0.44 mL, 8.28 mmol). The mixture was stirred at 20° C. for 1 h. The reaction was quenched with MeOH (5 mL). The solvent was removed in vacuo. The residue was washed with saturated NaHCO₃(20 mL), extracted with EtOAc (20 mL×2). The organics were collected and concentrated to give the desired compound (1.5 g, crude) as colorless oil, which was used directly for the next step without further purification.

Step 7. Preparation of compound (S)-2-((2S,3S,4S,5S)-6-acetoxy-3,4,5-trihydroxytetrahydro-2H-pyran-2-yl)-2-fluoroethyl stearate.

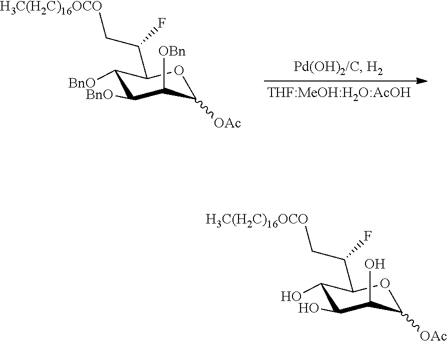

To a solution of compound obtained from step 6 above (1.5 g, 1.90 mmol) in MeOH (20 mL), AcOH (0.5 mL), THF (10 mL) and H₂O (2 mL) was added Pd(OH)₂/C (450 mg, 640.87 μmol, 20% purity). The mixture was stirred under H₂ at 45 psi at 25° C. for 12 h. The catalyst was filtered off. The filtrate was collected and concentrated to give crude product (1.2 g, crude) as yellow oil, which was re-dissolved in MeOH (20 mL), THF (10 mL), H₂O (5 mL) and AcOH (2 mL). Pd(OH)₂/C (470 mg, 669.35 μmol, 20% purity) was added. The mixture was stirred at 25° C. under H₂ at 45 psi for 12 h. The catalyst was filtered off. The filtrate was collected and concentrated to give the desired compound (1.2 g, crude) as colorless oil, which was used directly for the next step without further purification.

Step 8. Preparation of compound (3S,4S,5S,6S)-6-((S)-1-fluoro-2-(stearoyloxy)ethyl)tetrahydro-2H-pyran-2,3,4,5-tetrayl tetraacetate.

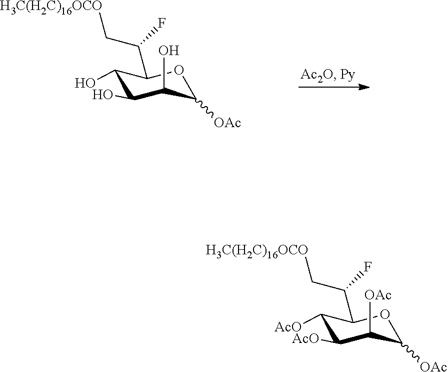

To a solution of compound obtained from step 7 (1.2 g, 2.30 mmol) in pyridine (10 mL) was added Ac₂O (1.1 mL, 11.52 mmol), DMAP (140 mg, 1.15 mmol). The mixture was stirred at 25° C. for 30 min. The reaction was quenched with MeOH (10 mL). The solvent was removed in vacuo. The residue was washed with 1N HCl (20 mL×2), extracted with EtOAc (30 mL). The organics were collected and concentrated. The residue was purified by column (PE:EA=1:0-2.5:1) to give the desired compound (0.95 g, yield: 47%) as colorless oil.

¹H NMR (400 MHz, CDCl₃) δ 6.10-6.05 (m, 1H), 5.52 (t, J=10.4 Hz, 1H), 5.36-5.29 (m, 1H), 5.25-5.21 (m, 1H), 4.72-4.66 (m, 0.5H), 4.60-4.54 (m, 0.5H), 4.41-4.20 (m, 2H), 4.14-4.06 (m, 1H), 3.97-3.93 (m, 0.5H), 3.92-3.87 (m, 0.5H), 2.31 (t, J=7.2 Hz, 2H), 2.17 (s, 3H), 2.15 (s, 3H), 2.05 (s, 3H), 1.99 (s, 3H), 1.64-1.58 (m, 2H), 1.28-1.22 (m, 28H), 0.88-0.83 (m, 3H).

Step 9. Preparation of compound (2S,3S,4S,5S)-2-((S)-1-fluoro-2-(stearoyloxy)ethyl)-6-hydroxytetrahydro-2H-pyran-3,4,5-triyl triacetate.

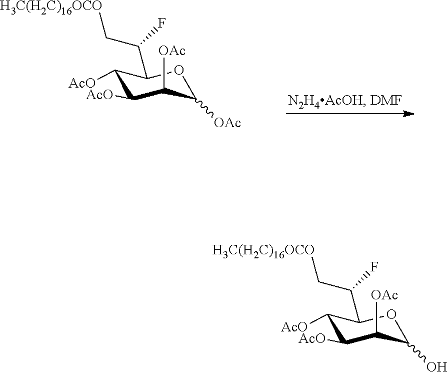

To a solution of compound obtained from step 8 above (950 mg, 1.47 mmol) in DMF (5 mL) was added hydrazine acetate (203 mg, 2.20 mmol). The mixture was stirred at 25° C. for 1 h. The mixture was washed with H₂O (30 mL), extracted with EtOAc (40 mL). The organics were collected and concentrated. The residue was purified by column (PE:EA=1:0 ˜1:1) to give the desired compound (800 mg, crude) as colorless oil, which was used directly for the next step without further purification.

Step 10. Preparation of compound (2S,3S,4S,5S,6S)-2-((diphenoxyphosphoryl)oxy)-6-((S)-1-fluoro-2-(stearoyloxy)ethyl)tetrahydro-2H-pyran-3,4,5-triyl triacetate.

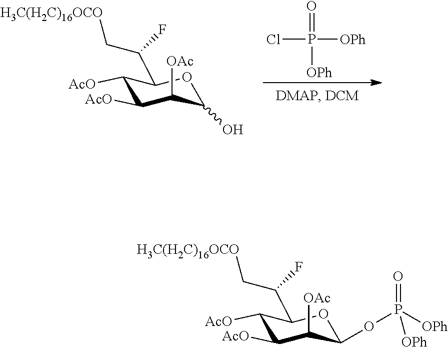

Compound diphenyl phosphorochloridate (1.07 g, 3.97 mmol) in DCM (30 mL) was added dropwise to a solution of compound obtained from step 9 above (800 mg, 1.32 mmol) and DMAP (808 mg, 6.61 mmol) in DCM (30 mL) over a period of 2 h. The mixture was stirred at 25° C. for 48 h. The mixture was washed with saturated NaHCO₃(30 mL). The organics were collected and concentrated. The residue was purified by column (PE:EA=1:0 ˜1:1) to give the desired compound (690 mg, yield: 57%) as colorless oil.

¹H NMR (400 MHz, CDCl₃) δ 7.38-7.29 (m, 4H), 7.28-7.25 (m, 2H), 7.23-7.11 (m, 4H), 5.56-5.52 (m, 1H), 5.51-5.44 (m, 2H), 5.07-5.02 (m, 1H), 4.72-4.66 (m, 0.5H), 4.60-4.54 (m, 0.5H), 4.28-4.19 (m, 2H), 3.73-3.62 (m, 1H), 2.34-2.28 (m, 2H), 2.09 (s, 3H), 2.04 (s, 3H), 1.97 (s, 3H), 1.63-1.58 (m, 2H), 1.28-1.23 (m, 28H), 0.89-0.83 (m, 3H).

Step 11. Preparation of compound (2S,3S,4S,5S,6S)-2-((S)-1-fluoro-2-(stearoyloxy)ethyl)-6-(phosphonooxy)tetrahydro-2H-pyran-3,4,5-triyl triacetate.

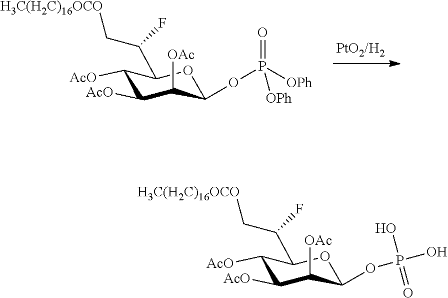

To a solution of compound obtained from step 10 above (690 mg, 824.46 μmol) in EtOH (20 mL) and EtOAc (20 mL) was added PtO₂ (180 mg, 792.68 μmol). The mixture was stirred at 20° C. under H₂ at 15 psi for 24 h. The catalyst was filtered off. The filtrate was collected and concentrated in vacuo to give the desired compound (554 mg, yield: 98%) as white solid.

Step 12. Preparation of triethylamine salt of (2S,3S,4S,5S,6S)-3,4,5-triacetoxy-6-((S)-1-fluoro-2-(stearoyloxy)ethyl)tetrahydro-2H-pyran-2-yl phosphate.

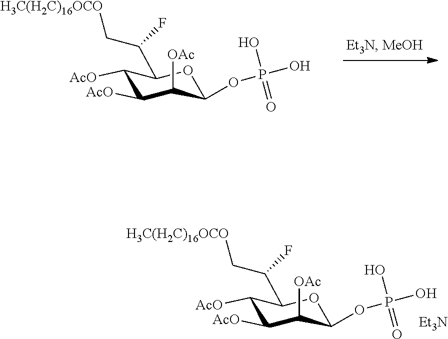

To a solution of compound obtained from step 11 above (554 mg, 809.09 mol) in MeOH (10 mL) was added TEA (0.24 mL, 1.62 mmol). The mixture was stirred at 20° C. for 30 min. The solvent was removed in vacuo to give the desired compound (595 mg, crude, 2TEA) as white solid, which was used directly for the next step without further purification.

¹H NMR (400 MHz, D₂O) δ 5.35-5.13 (m, 4H), 4.51-4.15 (m, 2H), 3.95-3.67 (m, 1H), 3.10-2.90 (m, 5H), 2.28-2.00 (m, 2H), 2.04 (s, 3H), 1.91-1.77 (m, 6H), 1.50-1.34 (m, 2H), 1.22-1.00 (m, 37H), 0.73-0.63 (m, 3H).

Step 13. Preparation of compound (2S,3S,4S,5S,6S)-2-((S)-1-fluoro-2-(stearoyloxy)ethyl)-6-((hydroxy(1H-imidazol-1-yl)phosphoryl)oxy)tetrahydro-2H-pyran-3,4,5-triyl triacetate.

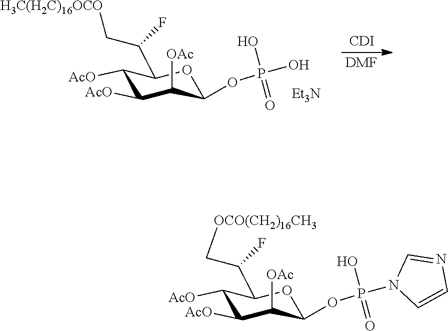

CDI (735 mg, 4.53 mmol) was added to a solution of compound obtained from step 12 above (300 mg, 438.13 μmol) in DMF (5 mL). The mixture was stirred at 20° C. for 4 h. According to reference, the reaction was complete after stirring for 4 h. MeOH (0.2 mL) was added to quench the reaction. The mixture was concentrated under reduced pressure to give the desired compound (1 g, crude) as colorless oil, which was used directly for the next step without further purification.

Step 14. Preparation of compound (2S,3S,4S,5S,6S)-2-(((((((3aR,4R,6R,6aR)-2,2-dimethyl-6-(6-(tritylamino)-9H-purin-9-yl)tetrahydrofuro[3,4-d] [1,3]dioxol-4-yl)methoxy)(hydroxy)phosphorothioyl)oxy)(hydroxy)phosphoryl)oxy)-6-((S)-1-fluoro-2-(stearoyloxy)ethyl)tetrahydro-2H-pyran-3,4,5-triyl triacetate.

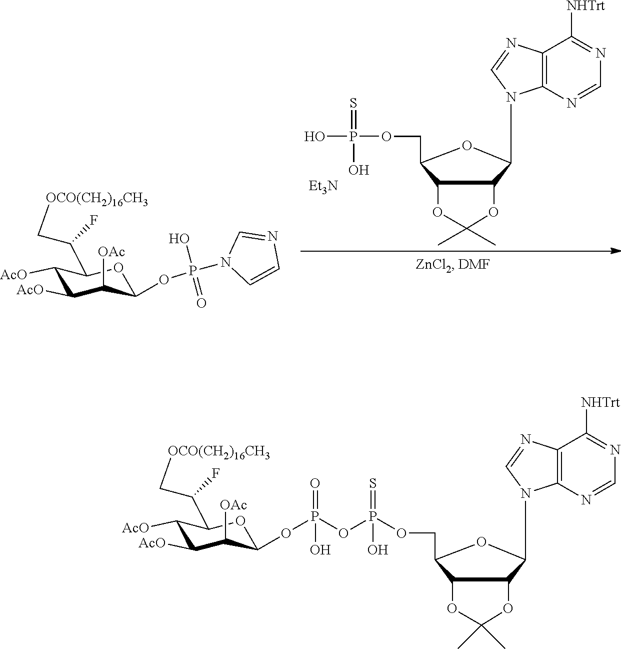

ZnCl₂ (740 mg, 5.43 mmol) was added to a solution of compound obtained from step 13 above (1 g, 1.36 mmol) and trimethylamine salt of O-(((3aR,4R,6R,6aR)-2,2-dimethyl-6-(6-(tritylamino)-9H-purin-9-yl)tetrahydrofuro[3,4-d][1,3]dioxol-4-yl)methyl) phosphorothioate (750 mg, 1.16 mmol) in DMF (5 mL). The mixture was stirred at 20° C. for 12 h. The solvent was removed in vacuo. The residue was purified by column (DCM: (MeOH:TEA=50:1)=10:1) to give the desired compound (900 mg, crude) as colorless oil, which was used directly for the next step without further purification.

MS (ESI) m/z (M+H)⁺: 1312.6.

Step 15. Preparation of (2S,3S,4S,5S,6S)-2-(((((((2R,3S,4R,5R)-5-(6-amino-9H-purin-9-yl)-3,4-dihydroxytetrahydrofuran-2-yl)methoxy)(hydroxy)phosphorothioyl)oxy)(hydroxy)phosphoryl)oxy)-6-((S)-1-fluoro-2-(stearoyloxy)ethyl)tetrahydro-2H-pyran-3,4,5-triyl triacetate.

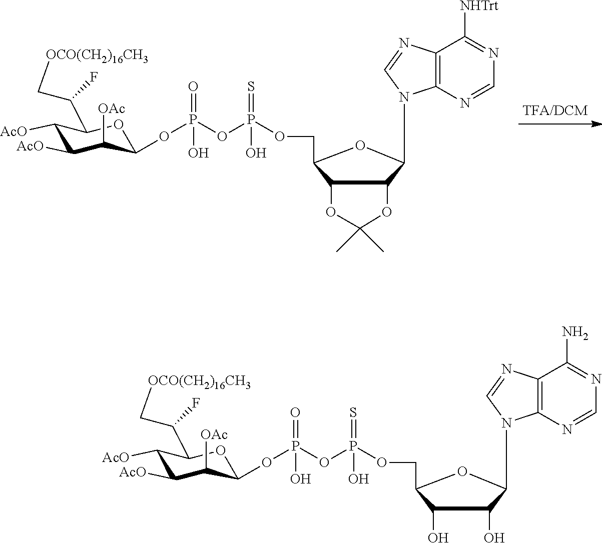

To a solution of the compound obtained in step 14 above (170 mg, 129.54 umol) in DCM (1.5 mL) was added TFA (0.45 mL, 6.08 mmol). The mixture was stirred at 25° C. for 2.5 h. The solution was quenched with TEA to pH=7. The solvent was removed in vacuo. The residue was purified by prep-HPLC (Neu) (column: Waters Xbridge 150*25 5 u; mobile phase: [water (10 mM NH₄HCO₃)-ACN]; B %: 42%-72%, 10 min) to give the desired compound (36 mg, yield: 26.74%) as white solid.

MS (ESI) m/z (M+H)⁺: 1030.5.

¹H NMR (400 MHz, CD₃OD) δ 8.78 (s, 0.3H), 8.71 (s, 0.6H), 8.27-8.19 (m, 1H), 6.10-6.04 (m, 1H), 5.74-5.68 (m, 1H), 5.67-5.59 (m, 1H), 5.39-5.31 (m, 1H), 5.25-5.17 (m, 1H), 4.76-4.71 (m, 1H), 4.64-4.43 (m, 4H), 4.42-4.23 (m, 3H), 3.95-3.82 (m, 1H), 2.33-2.25 (m, 2H), 2.16-2.10 (m, 3H), 2.02 (s, 3H), 1.94-1.89 (m, 3H), 1.60-1.49 (m, 2H), 1.31-1.25 (m, 28H), 0.91-0.85 (m, 3H).

### Compound A2

Preparation of compound 2S,3S,4S,5S,6S)-2-(((((((2R,3S,4R,5R)-5-(6-amino-9H-purin-9-yl)-3,4-dihydroxytetrahydrofuran-2-yl)methoxy)(hydroxy)phosphorothioyl)oxy)(hydroxy)phosphoryl)oxy)-6-((S)-2-(dodecanoyloxy)-1-fluoroethyl)tetrahydro-2H-pyran-3,4,5-triyl triacetate.

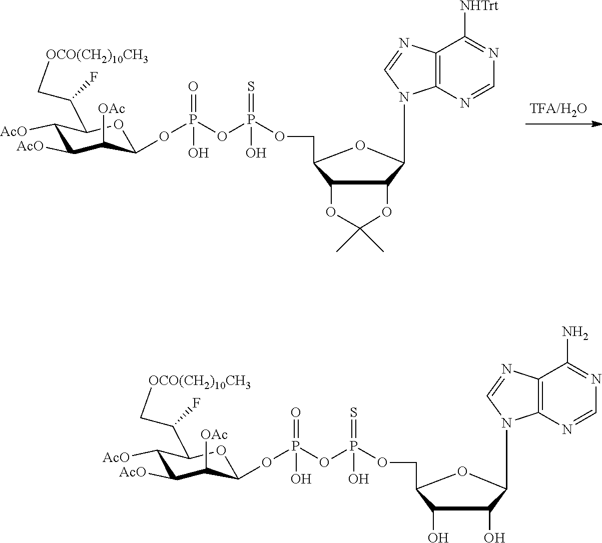

To the mixture of compound 2S,3S,4S,5S,6S)-2-(((((((3aR,4R,6R,6aR)-2,2-dimethyl-6-(6-(tritylamino)-9H-purin-9-yl)tetrahydrofuro[3,4-d][1,3]dioxol-4-yl)methoxy)(hydroxy)phosphorothioyl)oxy)(hydroxy)phosphoryl)oxy)-6-((S)-2-(dodecanoyloxy)-1-fluoroethyl)tetrahydro-2H-pyran-3,4,5-triyl triacetate (760 mg, 618.8 umol, synthesized with the same method described in compound A1) in H₂O (0.5 mL) was added TFA (1.16 g, 10.1 mmol, 0.8 mL). The mixture was stirred at 10° C. for 1 hr. The reaction was quenched with Et₃N (˜0.3 mL) to Ph=6. The mixture was dissolved in H₂O (1 mL) and methanol (3 mL). The residue was purified by Prep-HPLC (column: Waters Xbridge 150*25 5 u; mobile phase: [water (10 mM NH4HCO3)-ACN]; B %: 32%-52%, 10 min) twice. The desired compound (13.39 mg, 13.9 umol, yield: 2.2%) was obtained as a white solid.

MS (ESI) m/z (M+H)⁺: 946.2.

¹H NMR (400 MHz, CD₃OD) δ 8.78-8.71 (m, 0.7H), 8.61 (s, 0.3H), 8.23-8.22 (m, 1H), 6.09-6.06 (m, 1H), 5.72-5.56 (m, 2H), 5.38-5.33 (m, 1H), 5.23-5.18 (m, 1H), 4.75-4.27 (m, 4H), 3.93-3.79 (m, 1H), 2.31-2.26 (m, 2H), 2.12 (s, 3H), 2.02 (s, 3H), 1.91 (s, 3H), 1.55-1.52 (m, 2H), 1.26 (s, 20H), 0.88 (t, J=6.8 Hz, 3H).

### Compound A3

Preparation of compound (2S,3S,4S,5S,6S)-2-(((((((2R,3S,4R,5R)-5-(6-amino-9H-purin-9-yl)-3,4-dihydroxytetrahydrofuran-2-yl)methoxy)(hydroxy)phosphorothioyl)oxy)(hydroxy)phosphoryl)oxy)-6-((S)-1-fluoro-2-(tetradecanoyloxy)ethyl)tetrahydro-2H-pyran-3,4,5-triyl triacetate.

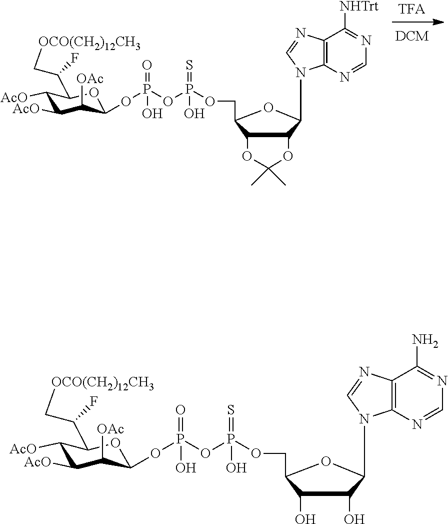

The mixture of compound (2S,3S,4S,5S,6S)-2-(((((((3aR,4R,6R,6aR)-2,2-dimethyl-6-(6-(tritylamino)-9H-purin-9-yl)tetrahydrofuro[3,4-d][1,3]dioxol-4-yl)methoxy)(hydroxy)phosphorothioyl)oxy)(hydroxy)phosphoryl)oxy)-6-((S)-1-fluoro-2-(tetradecanoyloxy)ethyl)tetrahydro-2H-pyran-3,4,5-triyl triacetate (630 mg, 501.49 umol, synthesized with the same method described in compound A1) and TFA (0.9 mL, 12.16 mmol) in DCM (3 mL) was stirred at 25° C. for 3 h. The reaction was adjusted to pH=6-7 with Et₃N. Then the solvent was removed under reduced pressure to give the crude. The crude was dissolved in H₂O (10 mL), extracted with EtOAc (10 mL), then the organic phase was concentrated to give crude product, which was purified by Pre-HPLC (Waters Xbridge 150*25 5 u, water (10 mM NH4HCO3)-ACN, 30%-60%). The desired compound (34 mg, yield: 6.8%) was obtained as white solid.

MS (ESI) m/z (M+H)⁺: 974.1.

¹H NMR (400 MHz, CD₃OD) δ 8.79 (s, 0.4H), 8.70 (s, 0.5H), 8.20 (s, 1H), 6.13 (dd, J=2.5, 5.8 Hz, 1H), 5.74 (dd, J=3.1, 9.9 Hz, 1H), 5.69 (d, J=3.0 Hz, 0.55H), 5.64 (d, J=3.0 Hz, 0.35H), 5.42-5.34 (m, 1H), 5.26-5.20 (m, 1H), 4.71-4.61 (m, 2H), 4.58-4.43 (m, 3H), 4.31-4.22 (m, 3H), 3.95-3.84 (m, 1H), 2.38-2.26 (m, 2H), 2.16 (s, 3H), 2.05 (s, 3H), 1.95 (s, 3H), 1.64-1.51 (m, 2H), 1.29 (s, 20H), 0.94-0.88 (m, 3H).

### Compound A4

Preparation of compound (2S,3S,4S,5S,6S)-2-(((((((2R,3S,4R,5R)-5-(6-amino-9H-purin-9-yl)-3,4-dihydroxytetrahydrofuran-2-yl)methoxy)(hydroxy)phosphorothioyl)oxy)(hydroxy)phosphoryl)oxy)-6-((S)-1-fluoro-2-(nonanoyloxy)ethyl)tetrahydro-2H-pyran-3,4,5-triyl triacetate.

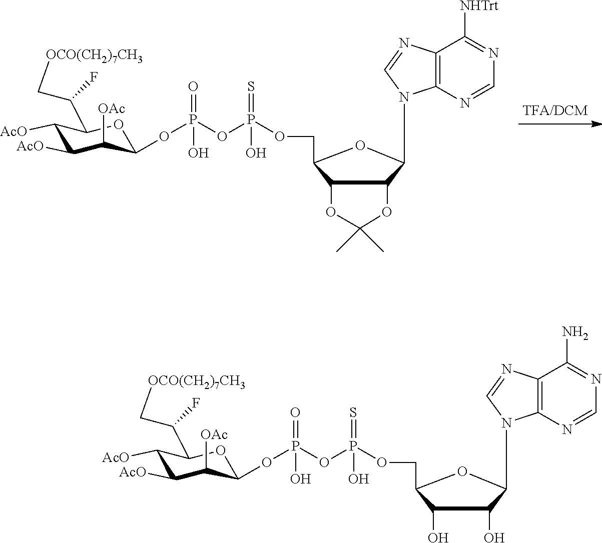

To a solution of compound (2S,3S,4S,5S,6S)-2-(((((((3aR,4R,6R,6aR)-2,2-dimethyl-6-(6-(tritylamino)-9H-purin-9-yl)tetrahydrofuro[3,4-d][1,3]dioxol-4-yl)methoxy)(hydroxy)phosphorothioyl)oxy)(hydroxy)phosphoryl)oxy)-6-((S)-1-fluoro-2-(nonanoyloxy)ethyl)tetrahydro-2H-pyran-3,4,5-triyl triacetate (310 mg, 155.24 umol, synthesized with the same method described in compound A1) in H₂O (1 mL) was added TFA (1.5 mL, 20.26 mmol). The mixture was stirred at 25° C. for 1.5 h. The solution was adjusted to pH=7 with NH₃.H₂O. The solution was purified by prep-HPLC (Neu) (column: Waters Xbridge 150*25 5 u; mobile phase: [water (10 mM NH4HCO3)-ACN]; B %: 16%-46%, 10 min.) to give the desired compound (25 mg, yield: 18%) as white solid.

MS (ESI) m/z (M+H)⁺: 904.6.

¹H NMR (400 MHz, CD₃OD) δ 8.76 (s, 0.4H), 8.66 (s, 0.6H), 8.18-8.16 (m, 1H), 6.11-6.08 (m, 1H), 5.74-5.68 (m, 1H), 5.67-5.59 (m, 1H), 5.39-5.31 (m, 1H), 5.25-5.17 (m, 1H), 4.76-4.71 (m, 1H), 4.64-4.43 (m, 4H), 4.42-4.23 (m, 3H), 3.95-3.82 (m, 1H), 2.33-2.25 (m, 2H), 2.16-2.10 (m, 3H), 2.02 (s, 3H), 1.94-1.89 (m, 3H), 1.60-1.49 (m, 2H), 1.31-1.25 (m, 10H), 0.91-0.85 (m, 3H).

### Compound A18

Step 1. Preparation of compound ((3aR,4R,6R,6aR)-6-(6-(benzylamino)-9H-purin-9-yl)-2,2-dimethyltetrahydrofuro[3,4-d][1,3]dioxol-4-yl)methanol.

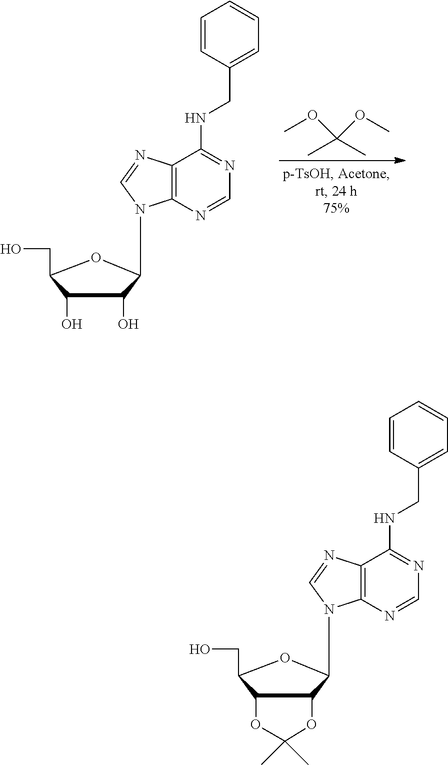

To a solution of compound (2R,3R,4S,5R)-2-(6-(benzylamino)-9H-purin-9-yl)-5-(hydroxymethyl)tetrahydrofuran-3,4-diol (5 g, 14.0 mmol) and 2,2-dimethoxypropane (14.5 g, 140.0 mmol) in acetone (200 mL) was added p-TsOH (4.8 g, 28.0 mmol). The mixture was stirred at 25° C. for 24 hr. After completion of the reaction, the mixture was cooled to 0° C. and quenched with sat. NaHCO₃(300 mL). The reaction mixture was extracted with EA (200 mL×3), the combined organic layers were washed with brine (200 mL), dried over Na₂SO₄, filtered and concentrated to give a residue, which was purified by silica gel column (PE:EA=5:1 to 1:2) to give the desired compound (4.2 g, yield: 75%) as a white solid.

MS (ESI) m/z (M+H)⁺: 398.

Step 2. Preparation of compound O-(((3aR,4R,6R,6aR)-6-(6-(benzylamino)-9H-purin-9-yl)-2,2-dimethyltetrahydrofuro[3,4-d][1,3]dioxol-4-yl)methyl) 0,0-di-tert-butyl phosphorothioate.

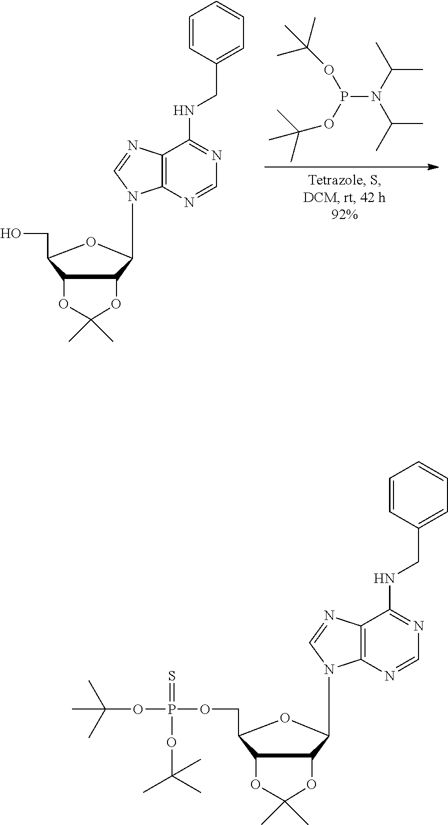

The mixture of compound obtained from step 1 above (2.1 g, 5.29 mmol) and Tetrazole (1.3 g, 18.5 mmol) in DCM (50 mL) was stirred at 25° C. for 5 min. Then di-tert-butyl diisopropylphosphoramidite (4.4 g, 15.9 mmol) was added. The mixture was stirred at 25° C. for 1 hr under N₂. To the above mixture was added sulfur (1.7 g, 52.9 mmol). The resulting mixture was stirred at 25° C. for 42 hr. After completion of the reaction, the mixture was directly concentrated under reduced pressure to give crude product, which was purified by silica gel column (PE:EA=1:0 to 2:1) to give the desired compound (2.95 g, yield 92%) as a yellow oil.

MS (ESI) m/z (M+H)⁺: 606

Step 3. Preparation of compound O-(((2R,3S,4R,5R)-5-(6-(benzylamino)-9H-purin-9-yl)-3,4-dihydroxytetrahydrofuran-2-yl)methyl) O,O-dihydrogen phosphorothioate.

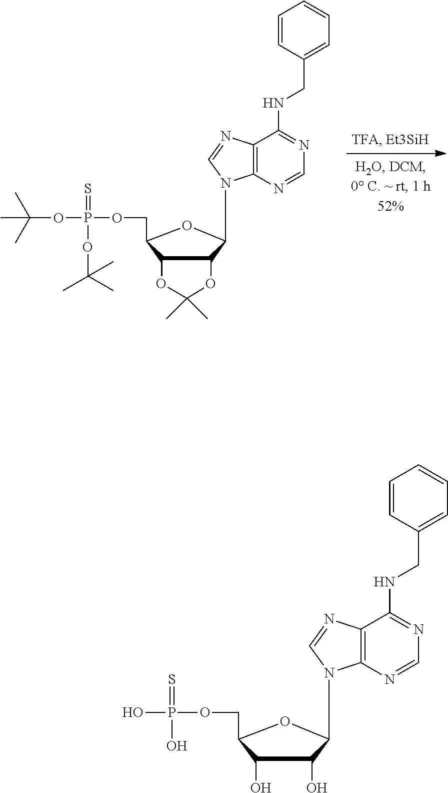

To the mixture of compound obtained from step 2 above (2.65 g, 4.38 mmol) and Et3SiH (10.16 g, 87.6 mmol) in H₂O (12 mL) and DCM (3 mL) was added TFA (18 mL) dropwise at 0° C. Then the mixture was stirred at 0° C. for 1 hr under N₂. After completion of the reaction, the reaction mixture was extracted with DCM (60 mL×2). The aqueous phase was concentrated to give crude product. The crude product was purified by silica gel column (DCM:MeOH=1:0 to 1:1, adding 0.5% Et₃N) to afford the desired compound (1.04 g, yield 52%) as a yellow gum.

MS (ESI) m/z (M+H)⁺: 454.

Step 4. Preparation of compound (2S,3S,4S,5S,6S)-2-((S)-2-acetoxy-1-fluoroethyl)-6-(((((((2R,3S,4R,5R)-5-(6-(benzylamino)-9H-purin-9-yl)-3,4-dihydroxytetrahydrofuran-2-yl)methoxy)(hydroxy)phosphorothioyl)oxy)(hydroxy)phosphoryl)oxy)tetrahydro-2H-pyran-3,4,5-triyl triacetate.

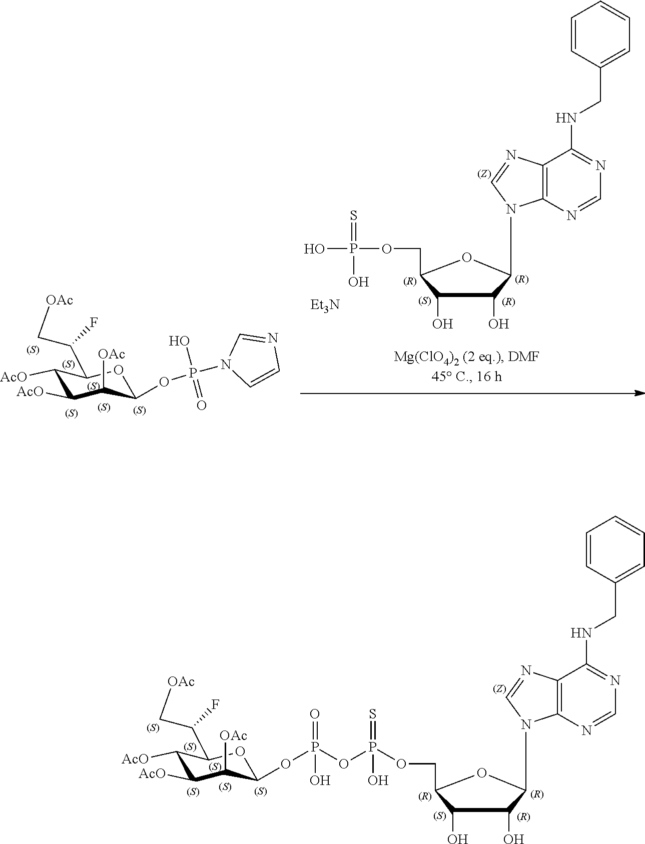

Mg(ClO4)₂ (97 mg, 0.434 mmol) was added to a solution of compound (2S,3S,4S,5S,6S)-2-((S)-2-acetoxy-1-fluoroethyl)-6-((hydroxy(1H-imidazol-1-yl)phosphoryl)oxy)tetrahydro-2H-pyran-3,4,5-triyl triacetate (112 mg, and compound obtained from step 3 above (100 mg, 0.22 mmol) in anhydrous DMF (4 mL) under N₂ atmosphere. The resulting mixture was stirred at 45° C. for 16 hr. After completion of the reaction, the mixture was added MTBE (20 Ml), then the solution was stirred at 25° C. for 0.5 hr. Turned off the stirring and let standing still for 10 min. Filtered out the filter residue which was purified by pre-HPLC (column: Waters Xbridge 150*25 5 u; mobile phase: [water (10 mM NH₄HCO₃)-ACN]; B %: 0%-30%, 10 min) to give the desired compound (20 mg, yield: 2%) as a white solid.

MS (ESI) m/z (M+H)⁺: 910.

¹H NMR (400 MHz, CD30D) δ 9.11 (d, 1H), 8.72 (s, 1H), 8.09 (d, 2H), 7.65 (m, 1H), 7.67-7.54 (m, 2H), 6.27 (m, 1H), 5.71-5.59 (m, 2H), 5.38-5.33 (m, 1H), 5.20-5.17 (m, 1H), 4.75 (m, 1H), 4.80-4.30 (m, 7H), 3.92-3.71 (m, 1H), 2.16-2.12 (m, 3H), 2.03-2.00 (m, 6H), 1.92 (s, 3H).

### Compound A26

Step 1. Preparation of compound (2R,3R,4R,5R)-2-(4-amino-1H-pyrazolo[3,4-d]pyrimidin-1-yl)-5-((benzoyloxy)methyl)tetrahydrofuran-3,4-diyl dibenzoate.

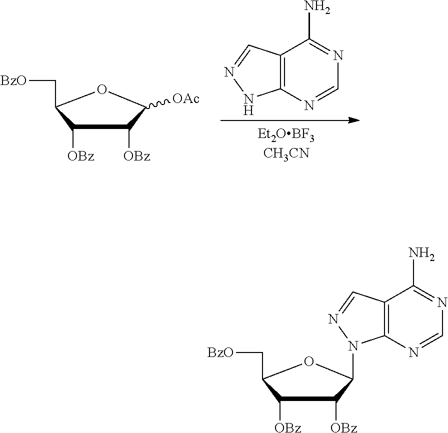

To a solution of compound (3R,4R,5R)-2-acetoxy-5-((benzoyloxy)methyl)tetrahydrofuran-3,4-diyl dibenzoate (10.10 g, 20.02 mmol) and 1H-pyrazolo[3,4-d]pyrimidin-4-amine (2 g, 14.80 mmol) in MeCN (100 mL) was added diethyloxonio(trifluoro)boranuide (3.05 g, 21.47 mmol, 2.65 mL). The mixture was stirred at 85° C. for 2 hr. The reaction mixture was concentrated to give a residue. The residue was purified by flash silica gel chromatography (PE:EA=1:0 to 0:1, DCM:MeOH=1:0 to 19:1). The desired compound (1.31 g, yield: 15.27%) was obtained as a yellow solid.

¹H NMR (400 MHz, DMSO-d6) δ 8.29 (s, 1H), 8.23 (s, 1H), 7.86-8.12 (m, 8H), 7.61-7.74 (m, 3H), 7.49 (dq, J=15.97, 7.89 Hz, 6H), 6.72 (d, J=2.76 Hz, 1H), 6.31 (dd, J=5.14, 2.89 Hz, 1H), 6.19-6.28 (m, 1H), 4.89 (dt, J=6.59, 3.61 Hz, 1H), 4.61-4.74 (m, 1H), 4.48-4.59 (m, 1H).

Step 2. Preparation of compound (2R,3R,4R,5R)-2-((benzoyloxy)methyl)-5-(4-(tritylamino)-1H-pyrazolo[3,4-d]pyrimidin-1-yl)tetrahydrofuran-3,4-diyl dibenzoate.

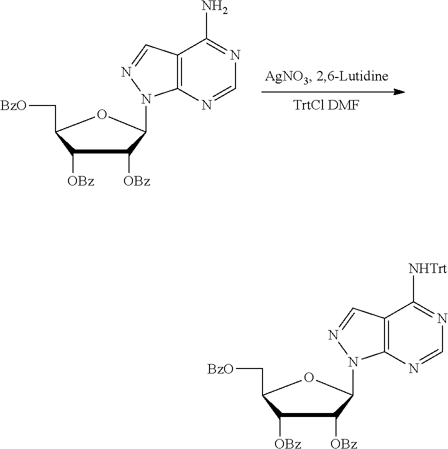

To a solution of compound obtained from step 1 above (16.68 g, 28.78 mmol) in DMF (100 mL) was added AgNO₃ (9.78 g, 57.56 mmol), 2,6-dimethylpyridine (6.17 g, 57.56 mmol, 6.70 mL) and TrtCl (16.05 g, 57.56 mmol). The mixture was stirred at 25° C. for 1 hr. The reaction mixture was diluted with EA (100 mL), filtered and the filter cake was washed with H₂O (60 mL), the filtrate was separated, the water layer was extracted with EA (60 mL×2), the combined organic layer was washed with HCl (1 N, 60 mL), brine (200 mL), dried over Na₂SO₄, filtered and concentrated to give a residue. The residue was purified by flash silica gel chromatography (PE:EA=1:0 to 1:1). The desired compound (18.40 g, yield: 77.79%) was obtained as a pale yellow oil.

MS (ESI) m/z (M+H)⁺: 822.2.

¹H NMR (400 MHz, CDCl3) δ 8.22 (br s, 1H), 7.94-8.06 (m, 7H), 7.89 (br d, J=7.78 Hz, 3H), 7.45-7.58 (m, 5H), 7.29-7.41 (m, 9H), 7.25 (s, 5H), 6.77 (s, 2H), 6.27 (br s, 3H), 4.46-4.81 (m, 4H).

Step 3. Preparation of compound (2R,3S,4R,5R)-2-(hydroxymethyl)-5-(4-(tritylamino)-1H-pyrazolo[3,4-d]pyrimidin-1-yl)tetrahydrofuran-3,4-diol.

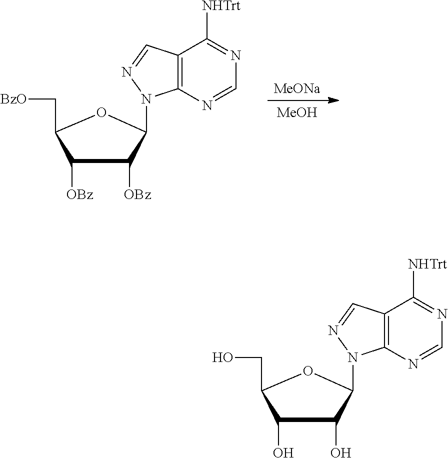

To a solution of compound obtained from step 2 above (18.40 g, 22.39 mmol) in MeOH (100 mL) was added NaOMe (3 g, 55.53 mmol) until the pH reached 11. The mixture was stirred at 60° C. for 1 hr. The reaction mixture was concentrated, then DCM (100 mL) was added and filtered, the filtrate was concentrated to give a residue. The residue was purified by flash silica gel chromatography (PE:EA=1:0 to 0:1, DCM:MeOH=1:0 to 5:1). The desired compound (9.85 g, yield: 85.96%) was obtained as a white solid.

MS (ESI) m/z (M+H)⁺: 510.1.

¹H NMR (400 MHz, DMSO-d6) δ 9.05 (br s, 1H), 8.56 (br s, 1H), 7.88 (s, 1H), 7.14-7.40 (m, 15H), 6.05 (d, J=4.77 Hz, 1H), 5.33 (d, J=5.77 Hz, 1H), 5.12 (d, J=5.52 Hz, 1H), 4.81 (t, J=5.77 Hz, 1H), 4.62 (q, J=5.27 Hz, 1H), 4.19 (q, J=4.94 Hz, 1H), 3.88 (q, J=4.68 Hz, 1H), 3.50-3.59 (m, 1H), 3.40 (dt, J=11.86, 5.99 Hz, 1H).

Step 4. Preparation of compound ((3aR,4R,6R,6aR)-2,2-dimethyl-6-(4-(tritylamino)-1H-pyrazolo[3,4-d]pyrimidin-1-yl)tetrahydrofuro[3,4-d][1,3]dioxol-4-yl)methanol.

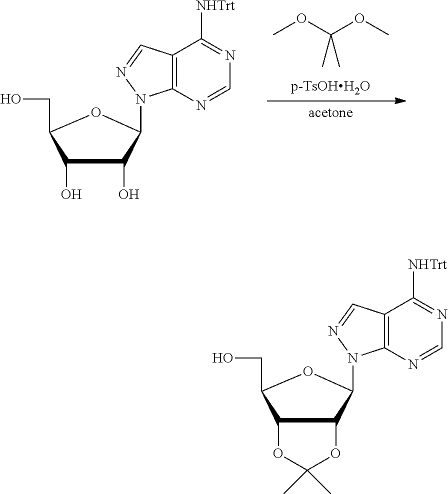

To a solution of compound obtained from step 3 above (5.85 g, 11.48 mmol) and 2,2-dimethoxypropane (11.96 g, 114.81 mmol, 14.07 mL) in ACETONE (100 mL) was added 4-methylbenzenesulfonic acid; hydrate (2.62 g, 13.78 mmol). The mixture was stirred at 25° C. for 2 hr. The reaction mixture was quenched with sat. NaHCO₃ (200 mL). The reaction mixture was extracted with EA (100 mL×3), the combined organic layers were washed with brine (200 mL), dried over Na₂SO₄, filtered and concentrated to give a residue. The residue was purified by flash silica gel chromatography (PE:EA=1:0 to 0:1, DCM:MeOH=1:0 to 10:1). Compound 5 (3.90 g, yield: 59.08%) was obtained as a white solid.

MS (ESI) m/z (M+H)⁺: 550.2.

¹H NMR (400 MHz, DMSO-d6) δ 9.11 (s, 1H), 8.60 (br s, 1H), 7.91 (s, 1H), 7.16-7.36 (m, 15H), 6.27 (s, 1H), 5.30 (dd, J=6.02, 1.51 Hz, 1H), 4.85-5.01 (m, 2H), 4.12 (q, J=5.27 Hz, 2H), 3.45-3.57 (m, 1H), 1.50 (s, 3H), 1.32 (s, 3H).

Step 5. Preparation of compound ((3aR,4R,6R,6aR)-2,2-dimethyl-6-(4-(tritylamino)-1H-pyrazolo[3,4-d]pyrimidin-1-yl)tetrahydrofuro[3,4-d][1,3]dioxol-4-yl)methyl phosphonate trimethylamine salt.

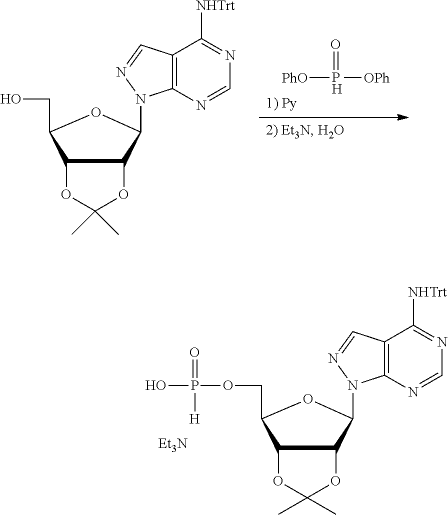

To a solution of compound obtained from step 4 above (3.90 g, 7.10 mmol) in Py (30 mL) was added phenoxyphosphonoyloxybenzene (4.99 g, 21.29 mmol, 4.09 mL), the mixture was stirred at 25° C. for 2 hr. Then Et₃N (3.59 g, 35.48 mmol, 4.94 mL) and H₂O (1.28 g, 70.96 mmol, 1.28 mL) was added, the mixture was stirred at 25° C. for 0.5 hr. The reaction mixture was concentrated to give a residue. The residue was purified by flash silica gel chromatography (DCM:MeOH=1:0 to 5:1, adding 0.5% Et₃N). The desired compound (5.91 g, crude, Et₃N salt) was obtained as a yellow syrup.

MS (ESI) m/z (M+H)⁺: 614.2.

Step 6. Preparation of compound O-(((3aR,4R,6R,6aR)-2,2-dimethyl-6-(4-(tritylamino)-1H-pyrazolo[3,4-d]pyrimidin-1-yl)tetrahydrofuro[3,4-d][1,3]dioxol-4-yl)methyl) phosphorothioate.

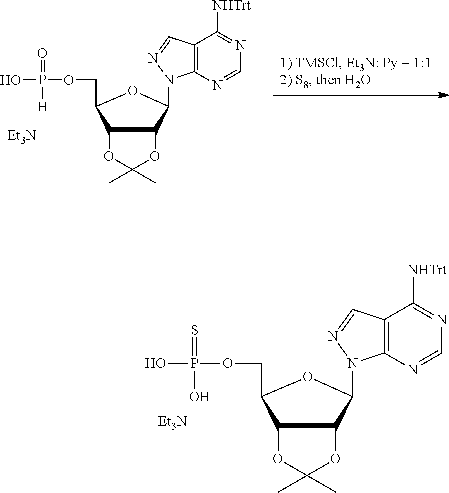

To a solution of compound obtained form step 5 above (5.91 g, 8.27 mmol, Et₃N) in Py (20 mL) and Et₃N (20 mL) was added TMSCl (6.16 g, 56.73 mmol, 7.20 mL) dropwise over 15 min under nitrogen atmosphere. The mixture was stirred at 0° C. for 1 hr, and then S (2.27 g, 70.79 mmol) was added. The mixture was stirred at 0° C. for another 45 min. H2O (25.00 g, 1.39 mol, 25.00 mL) was added and the mixture was stirred at 0° C. for 5 min. The reaction mixture was filtered and concentrated to give a residue. The residue was purified by flash silica gel chromatography (DCM:MeOH=1:0 to 1:1, adding 0.5% Et₃N). The desired compound (14.41 g, yield: 90.78%, Et₃N) was obtained as a white solid.

MS (ESI) m/z (M+H)⁺: 646.1.

¹H NMR (400 MHz, DMSO-d6) δ 9.30 (br s, 1H), 8.72-8.76 (m, 1H), 8.08 (s, 1H), 7.26-7.59 (m, 15H), 6.87-6.98 (m, 1H), 6.45 (s, 1H), 5.43-5.55 (m, 1H), 5.17 (dd, J=6.02, 1.76 Hz, 1H), 4.42 (br t, J=6.40 Hz, 1H), 3.75-3.86 (m, 1H), 3.15 (q, J=7.28 Hz, 60H), 1.66 (s, 3H), 1.49 (s, 3H).

Step 7. Preparation of compound (2S,3S,4S,5S,6S)-2-((S)-2-acetoxy-1-fluoroethyl)-6-(((((((3aR,4R,6R,6aR)-2,2-dimethyl-6-(4-(tritylamino)-1H-pyrazolo[3,4-d]pyrimidin-1-yl)tetrahydrofuro[3,4-d][1,3]dioxol-4-yl)methoxy)(hydroxy)phosphorothioyl)oxy)(hydroxy)phosphoryl)oxy)tetrahydro-2H-pyran-3,4,5-triyl triacetate.

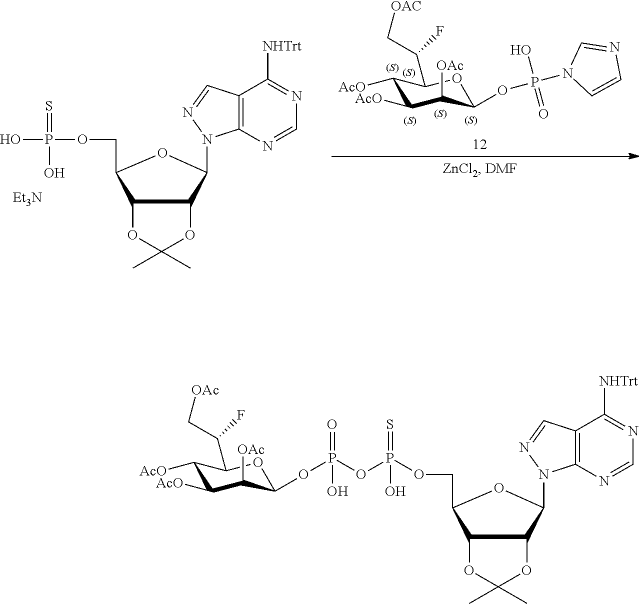

To a solution of imidazol-1-yl-[(2S,3R,5S)-3,4,5-triacetoxy-6-[(1S)-2-acetoxy-1-fluoro-ethyl]tetrahydropyran-2-yl]oxy-phosphinic acid (410 mg, 803.35 mol) and compound obtained from step 6 above (1.15 g, 803.35 mol, 45% purity) in DMF (5 mL) was added ZnCl₂ (1.31 g, 9.64 mmol). The mixture was stirred at 25° C. for 16 hr. The reaction was concentrated under reduced pressure to remove solvent. The residue was purified by silica gel chromatography (DCM/MeOH=20/1, 10/1, adding 0.5%). The desired (1.29 g, yield: 75.27%) was obtained as yellow syrups. MS (ESI) m/z (M+H)⁺: 1088.8.

Step 8. Preparation of (2S,3S,4S,5S,6S)-2-((S)-2-acetoxy-1-fluoroethyl)-6-(((((((2R,3S,4R,5R)-5-(4-amino-1H-pyrazolo[3,4-d]pyrimidin-1-yl)-3,4-dihydroxytetrahydrofuran-2-yl)methoxy)(hydroxy)phosphorothioyl)oxy)(hydroxy)phosphoryl)oxy)tetrahydro-2H-pyran-3,4,5-triyl triacetate (26A).

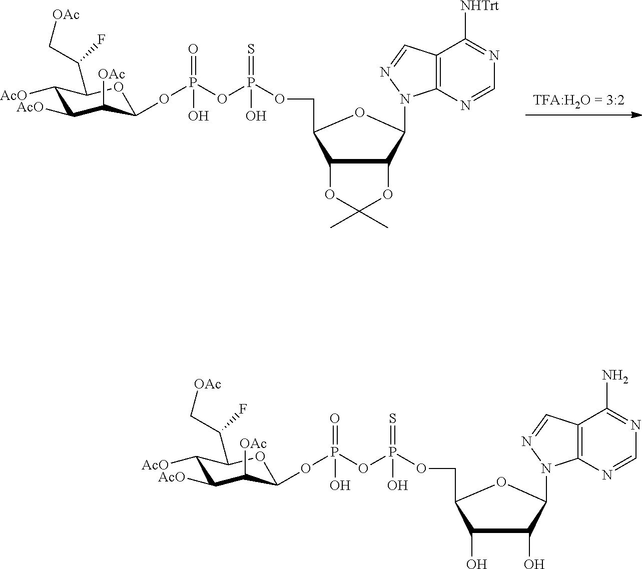

To a solution of compound obtained from step 7 above (120 mg, 110.30 μmol) in DCM (1.5 mL) was added TFA (770.00 mg, 6.75 mmol, 500 uL). Then the reaction was stirred at 25° C. for 2 h. The mixture was stirred at 25° C. for 4 hr. The reaction was quenched with TEA until pH=7. The crude product was purified by prep-HPLC (column: Phenomenex Gemini-NX 80*30 mm*3 um; mobile phase: [water (10 mM NH4HCO3)-ACN]; B %: 0%-30%, 9 min). The desire compound (10 mg, yield: 11.24%) was obtained as a white solid.

MS (ESI) (M+H)⁺: 806.0

¹H NMR (METHANOL-d4, 400 MHz): δ=8.22 (s, 1H), 8.17 (s, 1H), 6.31 (d, J=4.0 Hz, 1H), 5.71 (d, J=10.0 Hz, 1H), 5.62-5.68 (m, 1H), 5.35-5.42 (m, 1H), 5.24 (dd, J=10.3, 3.0 Hz, 1H), 4.75-4.85 (m, 1H), 4.61-4.70 (m, 2H), 4.07-4.57 (m, 5H), 3.86-3.98 (m, 1H), 2.17 (d, J=1.5 Hz, 3H), 2.03-2.10 (m, 6H), 1.96 ppm (s, 3H).

### Compound A27

Step 1. Preparation of compound (N-(9-((3aR,4R,6R,6aR)-6-(hydroxymethyl)-2,2-dimethyltetrahydrofuro[3,4-d][1,3]dioxol-4-yl)-9H-purin-6-yl)benzamide).

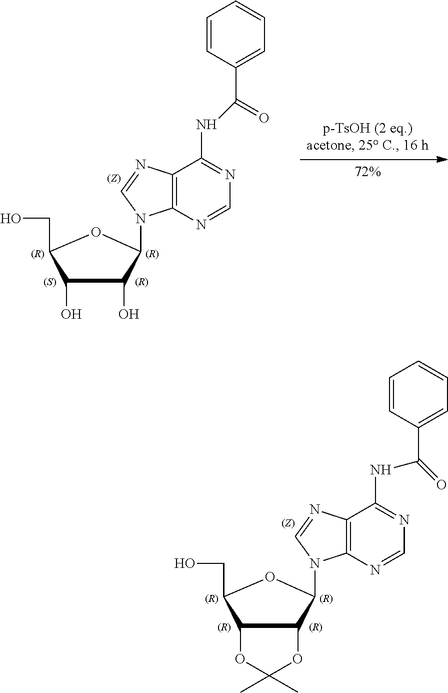

To a solution of compound N-(9-((2R,3R,4S,5R)-3,4-dihydroxy-5-(hydroxymethyl)tetrahydrofuran-2-yl)-9H-purin-6-yl)benzamide (5.00 g, 13.48 mmol) in acetone (150 mL) was added p-TsOH (4.64 g, 26.96 mmol). The mixture was stirred at 25° C. for 16 hr. After completion of the reaction, the mixture was cooled to 0° C. and quenched with sat. NaHCO₃(300 mL). The reaction mixture was extracted with EA (200 mL×3), the combined organic layers were washed with brine (200 mL), dried over Na₂SO₄, filtered and concentrated to give a residue, which was purified by silica gel column (DCM:MeOH=20:1 to 10:1) to give the desired compound (4.00 g, yield: 72%) as a white solid.

MS (ESI) m/z (M+H)⁺: 412.

Step 2. Preparation of compound (O-(((3aR,4R,6R,6aR)-6-(6-benzamido-9H-purin-9-yl)-2,2-dimethyltetrahydrofuro[3,4-d][1,3]dioxol-4-yl)methyl) 0,0-di-tert-butyl phosphorothioate).

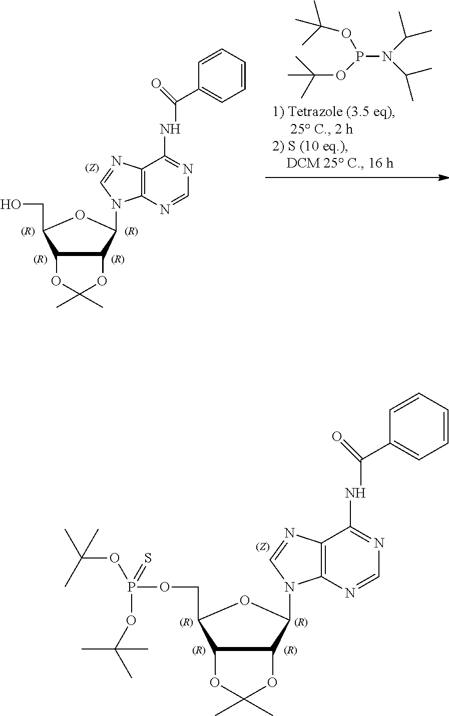

Tetrazole (1.51 g, 21.55 mmol) was added to a solution of compound obtained from step 1 above (2.53 g, 6.156 mmol) in DCM (50 mL). The resulting mixture was stirred at 25° C. for 5 min. Then compound di-tert-butyl diisopropylphosphoramidite (5.12 g, 18.47 mmol) was added. The resulting mixture was stirred at 25° C. for 1 hr. After completion of the reaction, sulfur (1.97 g, 61.56 mmol) was added, and the mixture was stirred at 25° C. overnight. After completion of the reaction, the mixture was directly concentrated under reduced pressure to give the crude product, which was purified by silica gel column (PE:EA=5:1 to 1:1) to give the desired compound (3.7 g, yield:97%).

MS (ESI) m/z (M+H)⁺: 620.

Step 3. Preparation of compound (O-(((2R,3S,4R,5R)-5-(6-benzamido-9H-purin-9-yl)-3,4-dihydroxytetrahydrofuran-2-yl)methyl) O,O-dihydrogen phosphorothioate).

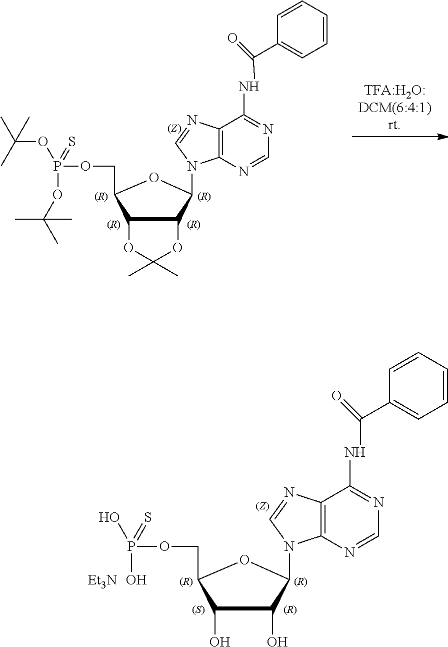

To a solution of compound obtained from step 2 above (1.00 g, 1.616 mmol) in TFA (6 mL), H₂O (4 mL) and DCM (1 mL) was stirred at rt. for 4 hr. After completion of the reaction, the mixture was concentrated under reduced pressure to give crude product, which was purified by silica gel column (DCM:MeOH=3:1 to 1:1, adding 0.5% Et₃N) to afford the desired compound (400 mg, yield 53%) as a pale yellow solid.

MS (ESI) m/z (M−H)⁺: 466.

Step 4. Preparation of compound ((2S,3S,4S,5S,6S)-2-((S)-2-acetoxy-1-fluoroethyl)-6-((hydroxy(1H-imidazol-1-yl)phosphoryl)oxy)tetrahydro-2H-pyran-3,4,5-triyl triacetate).

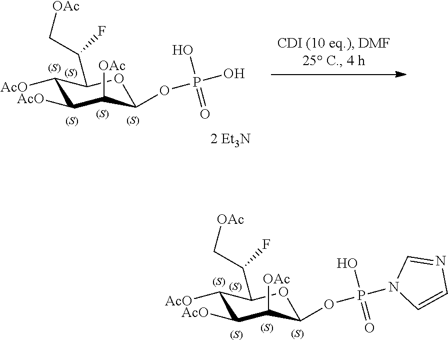

To the mixture of compound (2S,3S,4S,5S,6S)-2-((S)-2-acetoxy-1-fluoroethyl)-6-(phosphonooxy)tetrahydro-2H-pyran-3,4,5-triyl triacetate triethyl amine salt (100 mg, 0.217 mmol, Et₃N) in DCM (4 mL) was added CDI (88 mg, 0.542 mmol). The mixture was stirred at 25° C. for 5 hr. After completion of the reaction, MeOH (1 mL) was added to quench the reaction, and the mixture was concentrated under reduced pressure to give crude desired product (111 mg, crude), which was used directly in next step.

Step 5. Preparation of compound ((2S,3S,4S,5S,6S)-2-((S)-2-acetoxy-1-fluoroethyl)-6-(((((((2R,3S,4R,5R)-5-(6-benzamido-9H-purin-9-yl)-3,4-dihydroxytetrahydrofuran-2-yl)methoxy)(hydroxy)phosphorothioyl)oxy)(hydroxy)phosphoryl)oxy)tetrahydro-2H-pyran-3,4,5-triyl triacetate).

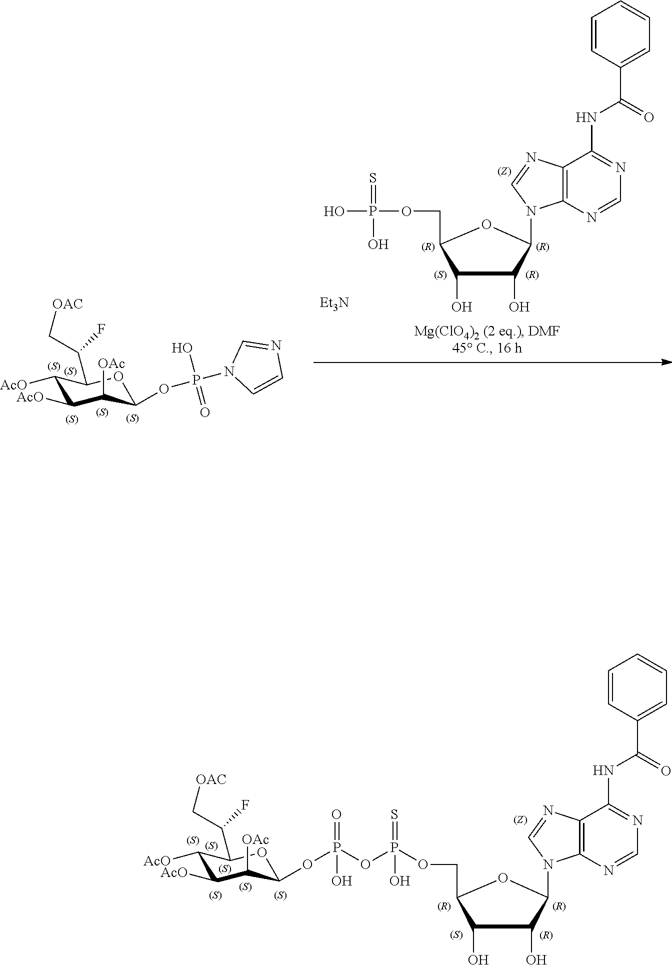

Mg(ClO4)₂ (97 mg, 0.434 mmol) was added to a solution of compound obtained from step 4 above (111 mg, crude) and compound obtained from step 3 above (101 mg, 0.217 mmol) in anhydrous DMF (4 mL) under N₂ atmosphere. The resulting mixture was stirred at 45° C. for 16 hr. After completion of the reaction, the mixture was added MTBE (20 Ml), then the solution was stirred at 25° C. for 0.5 hr. Turned off the stirring and let standing still for 10 min. Filtered out the filter residue which was purified by pre-HPLC (column: Waters Xbridge 150*25 5 u; mobile phase: [water (10 mM NH₄HCO₃)-ACN]; B %: 0%-30%, 10 min) to give the desired compound (5 mg, yield: 0.02%) as a white solid.

MS (ESI) m/z (M+H)⁺: 910.

¹H NMR (MeOD, 400 MHz): δ=9.09-9.14 d, J=21 Hz, 1H), 8.72 (s, 1H), 8.10 (d, J=7.6 Hz, 2H), 7.63-7.67 (m, 1H), 7.64-7.58 (m, 2H), 6.27 (t, J=2.4 Hz, 1H), 5.68-5.71 (m, 2H), 5.33-5.59 (m, 1H), 5.17-5.20 (m, 1H), 4.30-4.75 (m, 9H), 3.89 (m, 1H), 2.16-2.13 (m, 3H), 2.04-2.00 (m, 6H), 1.94 (s, 3H).

### Compound A28

Step 1. Preparation of compound (3aR,6R,6aR)-6-(hydroxymethyl)-2,2-dimethyltetrahydrofuro[3,4-d][1,3]dioxol-4-ol.

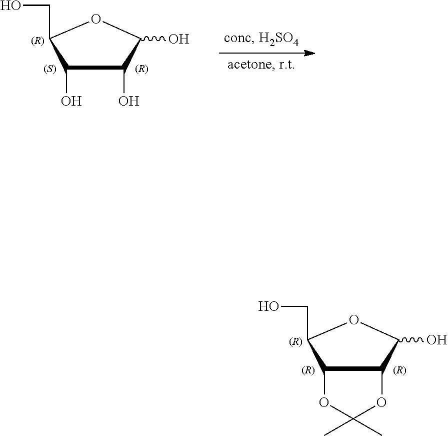

To a solution of (3R,4S,5R)-5-(hydroxymethyl)tetrahydrofuran-2,3,4-triol (10 g, 66.6 mmol) in acetone (100 mL) was added H₂SO₄ (3.55 mL, 66.6 mmol). Then the reaction was stirred at 25° C. for 2 h. The reaction was quenched with NaHCO₃ solid until pH=7. The mixture was filtered and the filtered liquid was concentrated under reduced pressure to remove solvent. The residue was purified by silica gel chromatography (Petroleum ether/Ethyl acetate=20/1, 1/1) to afford the desired compound (10 g, yield: 78.9%) as colorless oil.

¹H NMR (400 MHz, DMSO-d₆) δ 6.49 (d, J=5.0 Hz, 1H), 5.16 (d, J=4.8 Hz, 1H), 4.93 (t, J=5.6 Hz, 1H), 4.68 (d, J=5.8 Hz, 1H), 4.43 (d, J=6.0 Hz, 1H), 3.99 (dd, J=6.9, 5.1 Hz, 1H), 1.37 (s, 3H), 1.24 (s, 3H).

Step 2. Preparation of compound (3aR,6R,6aR)-6-(((tert-butyldiphenylsilyl)oxy)methyl)-2,2-dimethyltetrahydrofuro[3,4-d][1,3]dioxol-4-ol

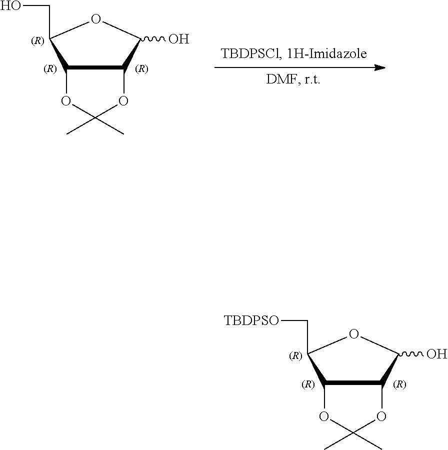

To a solution of compound obtained from step 1 above (10 g, 52.5 mmol) in DCM (100 mL) was added TEA (14.6 mL, 105.1 mmol), TBDPSCl (15.9 g, 57.84 mmol), DMAP (642.3 mg, 5.2 mmol). Then the reaction was stirred at 25° C. for 3 h. The reaction was quenched with H₂O (30 mL) and the organic layer was separated. The organic layer was washed with HCl (50 mL), brine (50 mL), dried over anhydrous Na₂SO₄, filtered and concentrated under reduced pressure. The residue was purified by silica gel chromatography (Petroleum ether/Ethyl acetate=20/1, 1/1) to afford the desired compound (20 g, yield: 88.75%) as colorless syrups.

¹H NMR (400 MHz, DMSO-d₆) δ 7.56-7.71 (m, 4H), 7.38-7.54 (m, 6H), 6.45 (d, J=4.0 Hz, 1H), 5.09-5.21 (m, 1H), 4.71 (d, J=6.0 Hz, 1H), 4.42 (d, J=6.0 Hz, 1H), 3.97-4.11 (m, 1H), 3.54-3.74 (m, 2H), 1.37 (s, 2H), 1.25 (s, 3H), 1.01 (s, 7H), 0.96-1.08 (m, 1H).

Step 3. Preparation of compound 2-((3aS,6R,6aR)-6-(((tert-butyldiphenylsilyl)oxy)methyl)-2,2-dimethyltetrahydrofuro[3,4-d][1,3]dioxol-4-yl)acetonitrile.

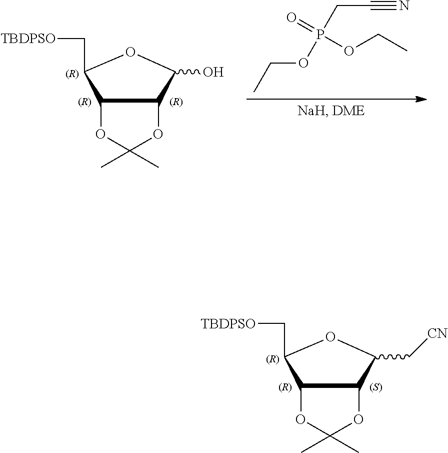

To a solution of 2-diethoxyphosphorylacetonitrile (4 g, 22.5 mmol) in DME (40 mL) was added NaH (903.1 mg, 22.5 mmol, 60% purity) at 0° C. and the reaction was stirred at 0° C. for 0.5 h. A solution of compound obtained from step 2 above (10.65 g, 24.8 mmol) in DME (100 mL) was added to the reaction and the reaction was stirred at 25° C. for 2 h. H₂O (80 mL) was added to the reaction and the mixture was extracted with EA (100 mL). The combined organic layer was washed with brine (100 mL), dried over anhydrous Na₂SO₄, filtered and concentrated under reduced pressure. The residue was purified by silica gel chromatography (Petroleum ether/Ethyl acetate=20/1, 10/1) to afford the desired compound (9.35 g, yield: 91.68%) as a white solid.

¹H NMR (400 MHz, DMSO-d₆) δ 7.59-7.73 (m, 4H), 7.41-7.53 (m, 6H), 4.81 (d, J=6.0 Hz, 1H), 4.65-4.75 (m, 1H), 4.23-4.54 (m, 1H), 4.06-4.15 (m, 1H), 3.62-3.81 (m, 2H), 2.64-2.99 (m, 2H), 1.39-1.48 (m, 3H), 1.29 (s, 3H), 1.01 (d, J=2.3 Hz, 9H).

Step 4. Preparation of compound 2-((3aS,6R,6aR)-6-(((tert-butyldiphenylsilyl)oxy)methyl)-2,2-dimethyltetrahydrofuro[3,4-d][1,3]dioxol-4-yl)-3-(dimethylamino)acrylonitrile.

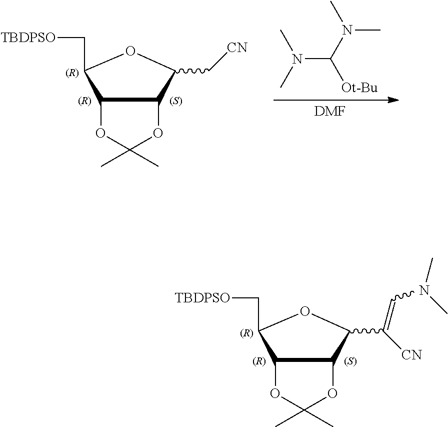

To a solution of compound obtained from step 3 above (10 g, 22.1 mmol) in DMF (100 mL) was added compound 1-tert-butoxy-N,N,N′,N′-tetramethylmethanediamine (30.87 g, 177.1 mmol). Then the reaction was stirred at 25° C. for 16 h. The reaction was diluted with H₂O (100 mL) and extracted with EA (100 mL). The organic layer was washed with H₂O (100 mL×2), brine (100 mL), dried over anhydrous Na₂SO₄, filtered and concentrated under reduced pressure to afford the desired compound (11.2 g, yield: 99.83%) as yellow syrups, which was used directly in next step.

Step 5. Preparation of compound 2-((3aS,6R,6aR)-6-(((tert-butyldiphenylsilyl)oxy)methyl)-2,2-dimethyltetrahydrofuro[3,4-d][1,3]dioxol-4-yl)-3-hydroxyacrylonitrile.

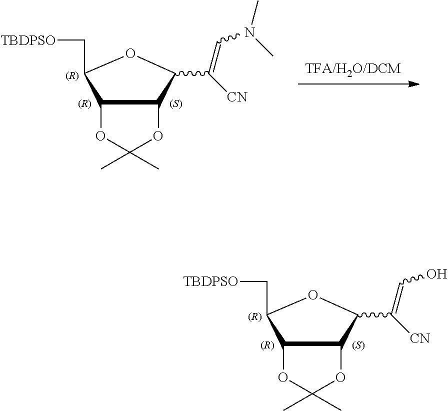

To a solution of compound obtained from step 4 above (11 g, 21.7 mmol) in CHCl₃ (200 mL) was added a solution of TFA (5.63 mL, 75.98 mmol) in H₂O (250 mL). The reaction was stirred at 25° C. for 16 h. The reaction was diluted with EA (200 mL) and H₂O (200 mL) was added to the mixture. The organic layer was separated and the organic layer was washed with H2O (200 mL×2), brine (200 mL), dried over anhydrous Na₂SO₄, filtered and concentrated under reduced pressure to afford the desired compound (10.41 g, crude) as yellow syrups, which was used directly in next step.

Step 6. Preparation of compound 2-((3aS,6R,6aR)-6-(((tert-butyldiphenylsilyl)oxy)methyl)-2,2-dimethyltetrahydrofuro[3,4-d][1,3]dioxol-4-yl)-3-(cyanomethoxy)acrylonitrile.

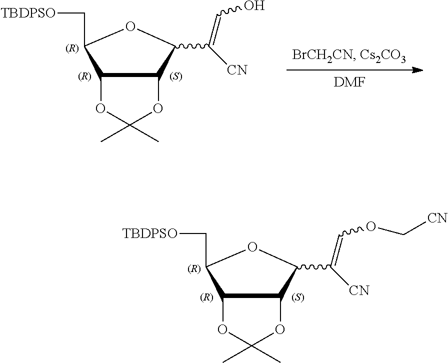

To a solution of compound obtained from step 5 above (10.4 g, 21.7 mmol,) in DMF (100 mL) was added Cs₂CO₃ (8.49 g, 26.0 mmol) and 2-bromoacetonitrile (3.12 g, 26.0 mmol). Then the reaction was stirred at 25° C. for 2 h. The reaction was diluted with H₂O (50 mL) and the mixture was extracted with EA (100 mL×2). The combined organic layer was washed with brine (50 mL), dried over anhydrous Na₂SO₄, filtered and concentrated under reduced pressure. The residue was purified by silica gel chromatography (Petroleum ether/Ethyl acetate=20/1, 5/1) to afford the desired compound (9.5 g, yield: 76.0%) as yellow syrups.

Step 7. Preparation of compound 3-amino-4-((3aS,4S,6R,6aR)-6-(((tert-butyldiphenylsilyl)oxy)methyl)-2,2-dimethyltetrahydrofuro[3,4-d][1,3]dioxol-4-yl)furan-2-carbonitrile.

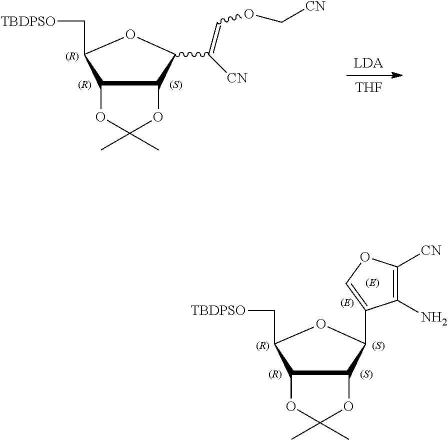

To a solution of compound obtained from step 6 above (10 g, 19.28 mmol) in THF (150 mL) was added LDA (2 M, 19.28 mL) at −70° C. and the reaction was stirred at −70° C. for 1 h. The reaction was quenched with H2O (30 mL) and the mixture was concentrated under reduced pressure. The aqueous layer was extracted with EA (30 mL×3), the combined organic layer was washed with brine (30 ml), dried over anhydrous Na₂SO₄, filtered and concentrated under reduced pressure. The residue was purified by silica gel chromatography (Petroleum ether/Ethyl acetate=20/1, 10/1) to afford the desired compound (2.6 g, yield: 26.0%) as yellow syrups.

MS (ESI) (M+Na⁺): 541.1.

¹H NMR (400 MHz, DMSO-d₆) δ 7.66 (s, 1H), 7.61 (br d, J=6.6 Hz, 3H), 7.36-7.50 (m, 6H), 5.74 (br s, 2H), 4.71-4.86 (m, 3H), 4.07 (br d, J=3.1 Hz, 1H), 3.72 (br d, J=4.4 Hz, 2H), 3.55-3.65 (m, 1H), 1.50 (s, 3H), 1.30 (s, 3H), 0.98 (s, 9H).

Step 8. Preparation of compound 7-((3aS,4S,6R,6aR)-6-(((tert-butyldiphenylsilyl)oxy)methyl)-2,2-dimethyltetrahydrofuro[3,4-d][1,3]dioxol-4-yl)furo[3,2-d]pyrimidin-4-amine.

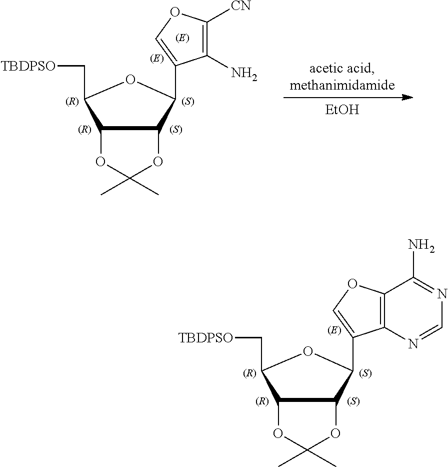

To a solution of compound obtained from step 7 above (4.00 g, 7.7 mmol) in EtOH (300 mL) was added acetic acid; methanimidamide (16.0 g, 154.2 mmol) and the reaction was stirred at 100° C. for 48 h. The reaction was concentrated under reduced pressure to remove solvent. The residue was purified by silica gel chromatography (Petroleum ether/Ethyl acetate=20/1, 1/1) to afford the desired compound (2 g, yield: 47.6%) as yellow syrups.

¹H NMR (400 MHz, DMSO-d₆) δ 8.19 (d, J=4.5 Hz, 2H), 7.58 (t, J=6.8 Hz, 4H), 7.40-7.47 (m, 2H), 7.31-7.39 (m, 6H), 5.14 (dd, J=6.4, 3.6 Hz, 1H), 5.06 (d, J=3.8 Hz, 1H), 4.84 (dd, J=6.3, 3.5 Hz, 1H), 4.08-4.16 (m, 1H), 3.74 (qd, J=10.7, 5.5 Hz, 2H), 1.90 (s, 1H), 1.51 (s, 3H), 1.30 (s, 3H), 0.95 (s, 9H).

Step 9. Preparation of compound 7-((3aS,4S,6R,6aR)-6-(((tert-butyldiphenylsilyl)oxy)methyl)-2,2-dimethyltetrahydrofuro[3,4-d][1,3]dioxol-4-yl)-N-tritylfuro[3,2-d]pyrimidin-4-amine.

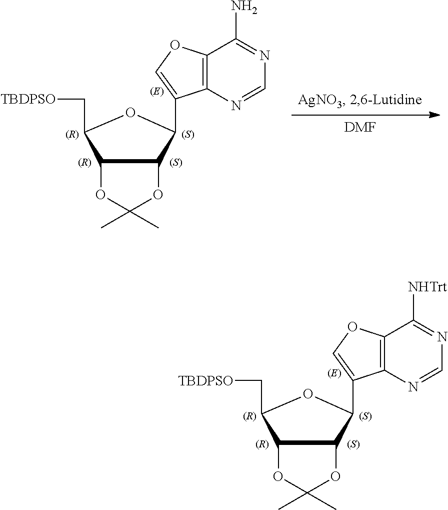

To a solution of compound obtained from step 8 above (1.2 g, 2.2 mmol) in DMF (10 mL) was added 2,6-dimethylpyridine (512.2 uL, 4.4 mmol) and TrtCl (1.23 g, 4.4 mmol), AgNO₃ (747.1 mg, 4.4 mmol). Then the reaction was stirred at 25° C. for 2 h. The reaction was diluted with EA and the mixture was filtered. The filtered liquid was concentrated under reduced pressure. The residue was purified by silica gel chromatography (Petroleum ether/Ethyl acetate=20/1, 1/1) to afford the desired compound (1.7 g, yield: 98.1%) as a white solid.

¹H NMR (400 MHz, DMSO-d₆) δ 8.32 (s, 1H), 8.22 (s, 1H), 7.92 (s, 1H), 7.51-7.62 (m, 4H), 7.29-7.43 (m, 12H), 7.26 (t, J=7.5 Hz, 5H), 7.15-7.21 (m, 3H), 5.76 (s, 1H), 5.07-5.12 (m, 1H), 5.03-5.06 (m, 1H), 4.79 (dd, J=6.3, 3.8 Hz, 1H), 4.06-4.13 (m, 1H), 3.66-3.80 (m, 2H), 1.49 (s, 3H), 1.27 (s, 3H), 0.93 (s, 9H).

Step 10. Preparation of compound ((3aR,4R,6S,6aS)-2,2-dimethyl-6-(4-(tritylamino)furo[3,2-d]pyrimidin-7-yl)tetrahydrofuro[3,4-d][1,3]dioxol-4-yl)methanol.

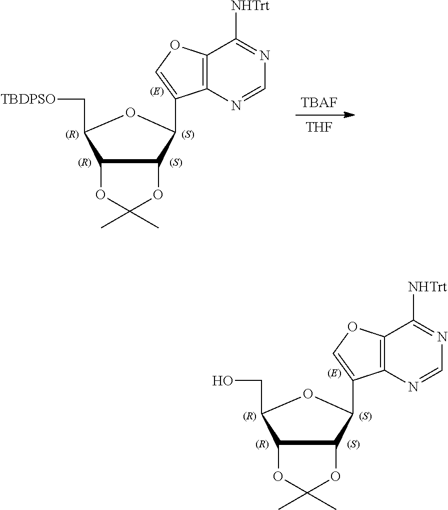

To a solution of compound obtained from step 9 above (1.7 g, 2.16 mmol) in THF (20 mL) was added TBAF (1 M, 3.24 mL). Then the reaction was stirred at 25° C. for 1 h. The reaction was diluted with H₂O (20 mL) and the mixture was extracted with EA (20 mL). The organic layer was washed with brine (20 mL×2), dried over anhydrous Na₂SO₄, filtered and concentrated under reduced pressure. The residue was purified by silica gel chromatography (Petroleum ether/Ethyl acetate=20/1, 2/1) to afford the desired compound (1.1 g, yield: 92.77%) as a white solid.

¹H NMR (400 MHz, DMSO-d₆) δ 8.37 (s, 1H), 8.31 (s, 1H), 7.96 (s, 1H), 7.32-7.39 (m, 6H), 7.26 (t, J=7.7 Hz, 6H), 7.16-7.22 (m, 3H), 5.26 (dd, J=6.8, 5.3 Hz, 1H), 4.94-5.00 (m, 2H), 4.70-4.79 (m, 1H), 4.04-4.08 (m, 1H), 3.44-3.55 (m, 2H), 1.50 (s, 3H), 1.27 (s, 3H).

Step 11. Preparation of compound ((3aR,4R,6S,6aS)-2,2-dimethyl-6-(4-(tritylamino)furo[3,2-d]pyrimidin-7-yl)tetrahydrofuro[3,4-d][1,3]dioxol-4-yl)methyl hydrogen phosphonate.

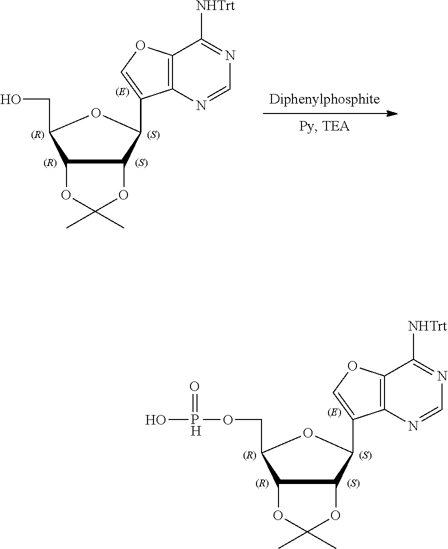

To a solution of compound obtained from step 10 above (1 g, 1.82 mmol) in Py (5 mL) was added diphenylphosphite (1.28 g, 5.46 mmol) at 25° C. for 2 h. TEA (1.27 mL, 9.10 mmol) and H₂O (327.78 uL, 18.19 mmol) was added to the reaction and the reaction was stirred at 25° C. for 0.5 h. The reaction was concentrated under reduced pressure to remove solvent. The residue was purified by silica gel chromatography (Petroleum ether/Ethyl acetate=20/1, 10/1) to afford the desired compound (1.1 g, yield 98.5%) as a white solid.

Step 12 Preparation of compound O-(((3aR,4R,6S,6aS)-2,2-dimethyl-6-(4-(tritylamino)furo[3,2-d]pyrimidin-7-yl)tetrahydrofuro[3,4-d][1,3]dioxol-4-yl)methyl) 0,0-dihydrogen phosphorothioate

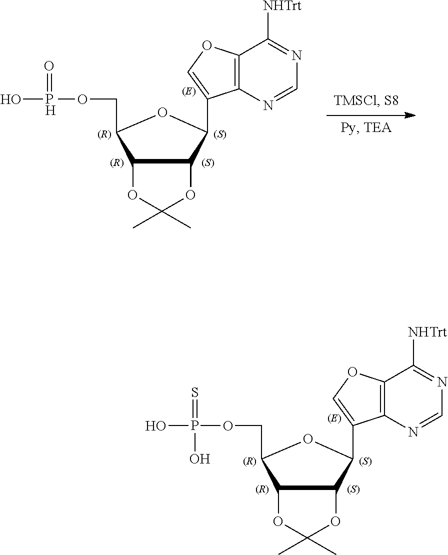

To a solution of compound obtained from step 11 above (1.1 g, 1.79 mmol,) in Py (10 mL) and TEA (10 mL) was added TMSCl (1.82 mL 14.3 mmol,) at 0° C. for 15 min. The reaction was stirred at 0° C. for 1 h and then S (574.83 mg, 17.93 mmol) was added to the reaction. The reaction was stirred at 0° C. for 45 min. The reaction was quenched with H₂O (1 mL) and the reaction was filtered. The filtered cake was washed with MeOH (5 mL×3). The filtered liquid was concentrated under reduced pressure to remove solvent. The residue was purified by silica gel chromatography (DCM/MeOH=20/1, 10/1) to afford the desired compound (2.5 g, yield: 93.36% 50% purity, Et₃N) as a white solid.

¹H NMR (400 MHz, DMSO-d₆) δ 8.33 (d, J=7.0 Hz, 2H), 7.99 (s, 1H), 7.34-7.40 (m, 6H), 7.27 (t, J=7.5 Hz, 6H), 7.15-7.23 (m, 4H), 5.09 (dd, J=6.3, 4.0 Hz, 1H), 5.02 (d, J=3.8 Hz, 1H), 4.79 (dd, J=6.3, 3.3 Hz, 1H), 4.09-4.17 (m, 2H), 3.79 (br t, J=6.7 Hz, 2H), 2.93 (q, J=7.1 Hz, 33H), 1.49 (s, 3H), 1.23-1.33 (m, 3H), 1.14 (t, J=7.2 Hz, 50H).

Step 13. Preparation of compound (2S,3S,4S,5S,6S)-2-((S)-2-acetoxy-1-fluoroethyl)-6-(((((((3aR,4R,6S,6aS)-2,2-dimethyl-6-(4-(tritylamino)furo[3,2-d]pyrimidin-7-yl)tetrahydrofuro[3,4-d][1,3]dioxol-4-yl)methoxy)(hydroxy)phosphorothioyl)oxy)(hydroxy)phosphoryl)oxy)tetrahydro-2H-pyran-3,4,5-triyl triacetate.

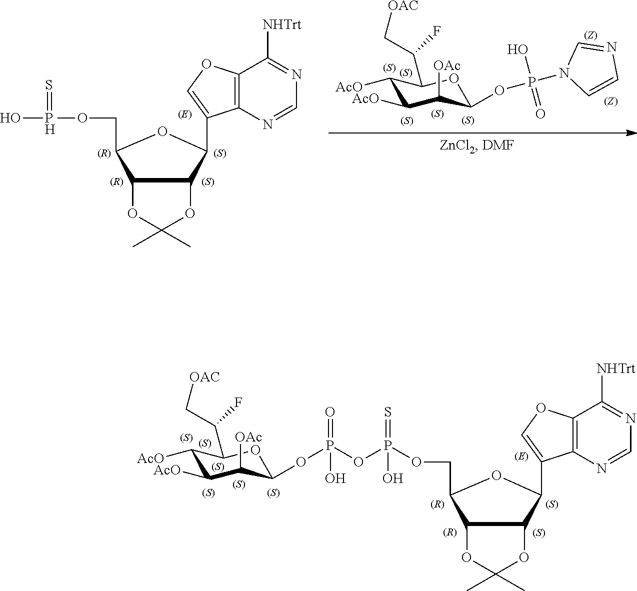

To a solution of compound obtained from step 12 above (443 mg, 868. umol) in DMF (10 mL) was added compound (2S,3S,4S,5S,6S)-2-((S)-2-acetoxy-1-fluoroethyl)-6-((hydroxy(1H-imidazol-1-yl)phosphoryl)oxy)tetrahydro-2H-pyran-3,4,5-triyl triacetate (1.12 g, 868.01 umol, 50% purity) and ZnCl₂ (1.42 g, 10.42 mmol). Then the reaction was stirred at 25° C. for 16 h. The reaction was concentrated under reduced pressure to remove solvent. The residue was purified by silica gel chromatography (DCM/MeOH=20/1, 10/1) to afford the desired compound (2 g, crude) as a white solid.

Step 14. Preparation of compound (2S,3S,4S,5S,6S)-2-((S)-2-acetoxy-1-fluoroethyl)-6-(((((((2R,3S,4R,5S)-5-(4-aminofuro[3,2-d]pyrimidin-7-yl)-3,4-dihydroxytetrahydrofuran-2-yl)methoxy)(hydroxy)phosphorothioyl)oxy)(hydroxy)phosphoryl)oxy)tetrahydro-2H-pyran-3,4,5-triyl triacetate

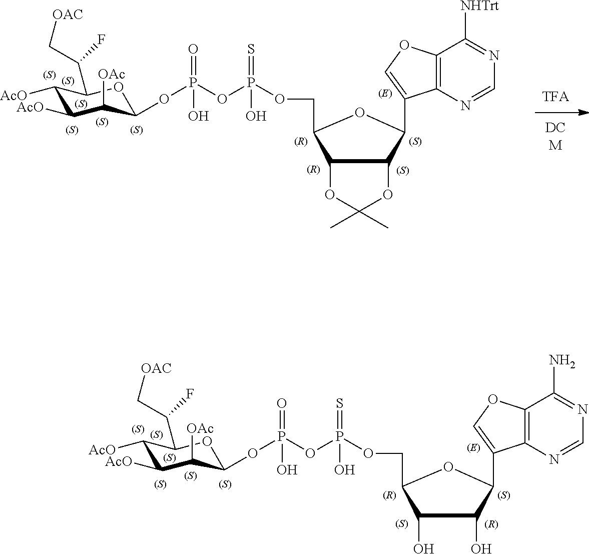

Compound obtained from step 13 above (200 mg, 183.83 umol) was added to the solution of TFA (1.5 mL 20.26 mmol,) in DCM (5 mL) and the reaction was stirred at 25° C. for 7 h. The reaction was treated with TEA until pH=6. The mixture was concentrated under reduced pressure. The crude product was purified by Prep-HPLC (water (10 mM NH4HCO3)-ACN]; B %: 1%-25%, 10 min) to afford the final compound (30.0 mg, yield: 20.2%) as a white solid.

MS (ESI) (M+H)⁺: 806.0.

¹H NMR (MeOD, 400 MHz): δ=8.20-8.34 (m, 2H), 7.71 (s, 1H), 7.06 (s, 1H), 5.69-5.78 (m, 1H), 5.58-5.68 (m, 1H), 5.32-5.45 (m, 1H), 5.22 (dd, J=10.0, 2.8 Hz, 1H), 5.11 (d, J=3.5 Hz, 1H), 4.60-4.79 (m, 1H), 4.34-4.58 (m, 2H), 4.13-4.34 (m, 5H), 3.80-3.97 (m, 1H), 3.12 (q, J=7.3 Hz, 1H), 2.13 (s, 3H), 2.01-2.08 (m, 6H), 1.94 (s, 3H), 1.10 ppm (t, J=7.2 Hz, 1H).

### Compound A30

Step 1. Preparation of compound 2-((3aS,6R,6aR)-6-(((tert-butyldiphenylsilyl)oxy)methyl)-2,2-dimethyltetrahydrofuro[3,4-d][1,3]dioxol-4-yl)-2-cyanovinyl methanesulfonate.

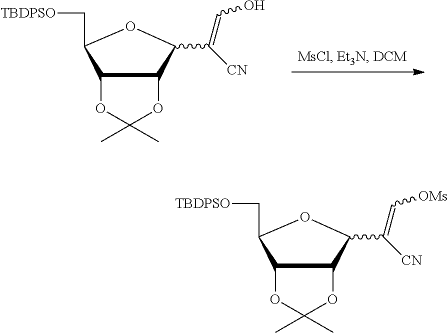

To a solution of 2-((3aS,6R,6aR)-6-(((tert-butyldiphenylsilyl)oxy)methyl)-2,2-dimethyltetrahydrofuro[3,4-d][1,3]dioxol-4-yl)-3-hydroxyacrylonitrile (1 g, 2.08 mmol) in DCM (16 mL) was added Et₃N (316.45 mg, 3.13 mmol, 435.29 μL) at 0° C., and then MsCl (358.24 mg, 3.13 mmol, 242.06 μL) in DCM (8 mL) was dropwise added over a period of 10 min. The mixture was stirred at 0° C. for 1 hr. The reaction mixture was diluted with DCM (10 mL) and then washed with water (20 mL×3). The combined organic layer was dried over Na₂SO₄, filtered and concentrated to give a residue. The desired compound (1.34 g, crude) was obtained as a brown oil which was used into the next step without further purification.

Step 2. Preparation of compound 3-amino-4-((3aS,4S,6R,6aR)-6-(((tert-butyldiphenylsilyl)oxy)methyl)-2,2-dimethyltetrahydrofuro[3,4-d][1,3]dioxol-4-yl)thiophene-2-carboxamide.

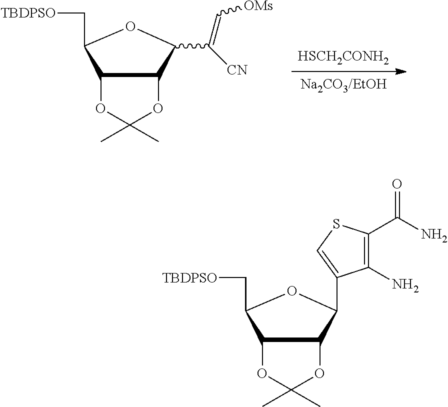

To a solution of compound obtained from step 1 above (1.34 g, 2.40 mmol) in EtOH (20 mL) was added 2-sulfanylacetamide (437.91 mg, 4.81 mmol) and Na₂CO₃ (509.30 mg, 4.81 mmol). The mixture was stirred at 85° C. for 6 hr. The reaction mixture was filtered and concentrated to give a residue. The residue was purified by flash silica gel chromatography (PE:EA=1:0 to 0:1). The desired compound (118 mg, yield: 8.18%) was obtained as a pale yellow oil.

MS (ESI) m/z (M+H)⁺: 553.2.

¹H NMR (400 MHz, CDCl3) δ 7.66 (br d, J=7.78 Hz, 5H), 7.36-7.51 (m, 7H), 7.10 (s, 1H), 5.24 (br s, 2H), 4.86 (dd, J=6.65, 3.64 Hz, 1H), 4.77 (d, J=6.02 Hz, 1H), 4.66-4.72 (m, 1H), 4.17 (q, J=3.26 Hz, 1H), 3.92-3.98 (m, 1H), 3.83-3.89 (m, 1H), 1.61 (s, 3H), 1.37 (s, 3H), 1.07 (s, 9H).

Step 3. Preparation of compound 3-amino-4-((3aS,4S,6R,6aR)-6-(hydroxymethyl)-2,2-dimethyltetrahydrofuro[3,4-d][1,3]dioxol-4-yl)thiophene-2-carboxamide.

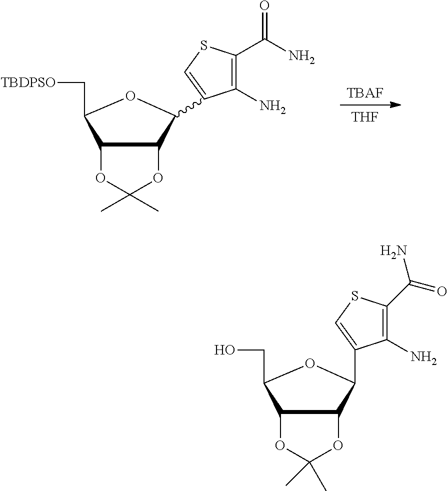

TBAF (1 M, 1.87 mL, 1.5 eq) was added to the solution of compound obtained from step 2 above (690 mg, 1.25 mmol) in THF (7 mL) and then stirred at 25° C. for 1 h. The reaction was diluted with EtOAc (50 mL), extracted with H₂O (20 mL×3) and brine (20 mL). The organic phase was combined and concentrated to give the residue. The residue was purified by silica gel chromatography (PE:EA=1:0 to 1:2). The desired compound (340 mg, yield: 86.64%) was obtained as yellow oil.

MS (ESI) m/z (M+Na⁺): 337.0.

¹H NMR (400 MHz, CDCl₃) δ 7.11 (s, 1H), 6.16 (br, s, 2H), 5.21 (s, 2H), 4.83-4.73 (m, 2H), 4.72-4.65 (m, 1H), 4.22-4.15 (m, 1H), 3.95-3.86 (m, 1H), 3.83-3.74 (m, 1H), 1.60 (s, 3H), 1.35 (s, 3H).

Step 4. Preparation of compound ((3aR,4R,6S,6aS)-6-(4-amino-5-carbamoylthiophen-3-yl)-2,2-dimethyltetrahydrofuro[3,4-d][1,3]dioxol-4-yl)methyl hydrogen phosphonate.

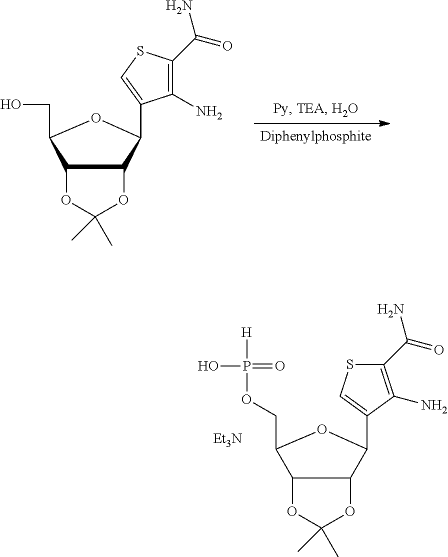

Diphenylphosphite (949.84 mg, 4.06 mmol, 778.56 μL, 3 eq) was added to the solution of compound obtained from step 3 above (425 mg, 1.35 mmol in pyridine (5 mL) and stirred at 20° C. for 4 h. Then TEA (684.02 mg, 6.76 mmol, 940.88 μL, 5 eq) and H₂O (243.56 mg, 13.52 mmol, 243.56 μL, 10 eq) was added and stirred at 25° C. for 0.5 h. The solvent was removed under reduced pressure to give the residue. The residue was purified by silica gel chromatography column (DCM:MeOH=1:0 to 1:1, adding 0.5% Et₃N). The desired compound (700 mg, yield: 93.80%, 1.7Et3N) was obtained as a yellow solid.

MS (ESI) m/z (M+H)⁺: 378.9.

Step 5. Preparation of compound O-(((3aR,4R,6S,6aS)-6-(4-amino-5-carbamoylthiophen-3-yl)-2,2-dimethyltetrahydrofuro[3,4-d][1,3]dioxol-4-yl)methyl) O,O-dihydrogen phosphorothioate.

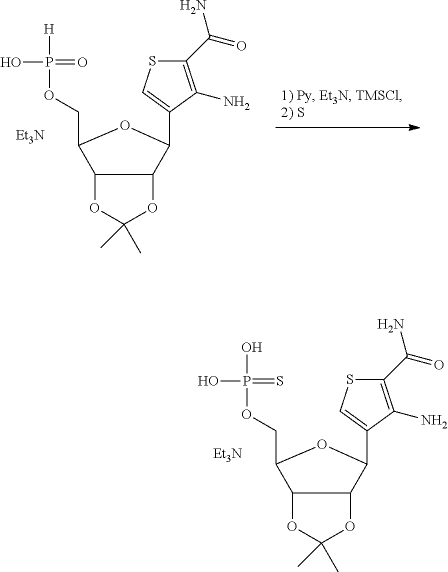

TMSCl (1.11 g, 10.18 mmol, 1.29 mL) was added dropwise to the solution of compound obtained from step 4 above (700 mg, 1.27 mmol, 1.7Et₃N) in pyridine (5 mL) and Et₃N (5 mL) during 10 min, then the reaction was stirred at 0° C. for 1 h. Sulfur (407.83 mg, 12.72 mmol, 10 eq) was added and stirred at 0° C. for 50 min. H₂O (160.40 mg, 8.90 mmol, 160.40 μL, 7 eq) was added to quench the reaction. The reaction was filtered and washed with MeOH (10 mL×3) to remove the solid, and the filtrated was concentrated under reduced pressure to give the residue. The residue was purified by silica gel chromatography (DCM:MeOH=1:0 to 1:1, adding 0.5% Et₃N). The desired compound (660 mg, yield: 63.22%, 50% purity) was obtained as a brown solid.

MS (ESI) m/z (M+H)⁺: 411.0.

Step 6. Preparation of compound (2S,3S,4S,5S,6S)-2-((S)-2-acetoxy-1-fluoroethyl)-6-(((((((3aR,4R,6S,6aS)-6-(4-amino-5-carbamoylthiophen-3-yl)-2,2-dimethyltetrahydrofuro[3,4-d][1,3]dioxol-4-yl)methoxy)(hydroxy)phosphorothioyl)oxy)(hydroxy)phosphoryl)oxy)tetrahydro-2H-pyran-3,4,5-triyl triacetate.

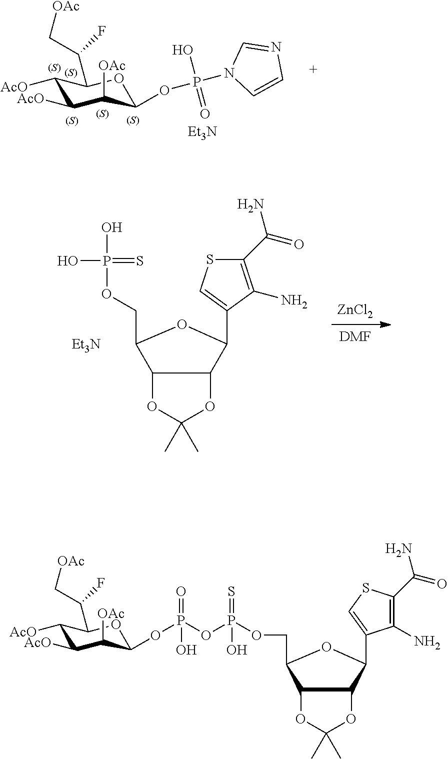

To the solution of imidazol-1-yl-[(2S,3R,5S)-3,4,5-triacetoxy-6-[(1S)-2-acetoxy-1-fluoro-ethyl]tetrahydropyran-2-yl]oxy-phosphinic acid (491.74 mg, 804.09 μmol, Et₃N) in DMF (5 mL) was added compound obtained from step 5 above (660 mg, 804.09 μmol, 50% purity) and then ZnCl₂ (1.64 g, 12.06 mmol, 564.92 μL) was added and stirred at 25° C. for 16 under N₂ atmosphere. The reaction was diluted with DCM (20 mL) and mixed with silica gel and then purified by silica gel chromatography (DCM:MeOH=1:0 to 1:1, adding 0.5% Et₃N). The desired compound (2.15 g, crude) was obtained as a brown solid.

MS (ESI) m/z (M+H)⁺: 853.1.

Step 7. Preparation of compound (2S,3S,4S,5S,6S)-2-((S)-2-acetoxy-1-fluoroethyl)-6-(((((((2R,3S,4R,5S)-5-(4-amino-5-carbamoylthiophen-3-yl)-3,4-dihydroxytetrahydrofuran-2-yl)methoxy)(hydroxy)phosphorothioyl)oxy)(hydroxy)phosphoryl)oxy)tetrahydro-2H-pyran-3,4,5-triyl triacetate.

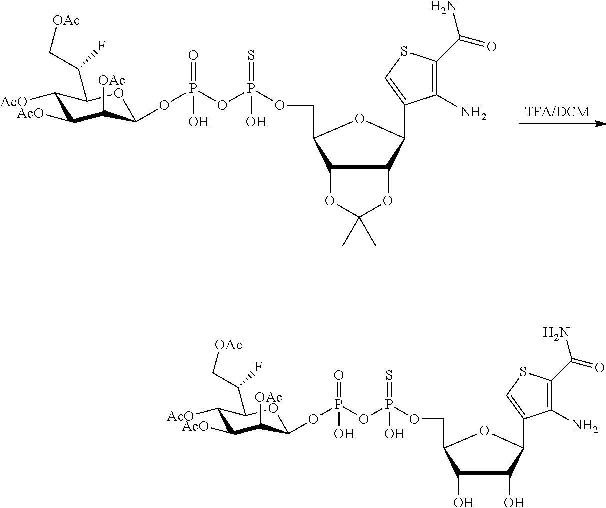

TFA (2 mL) was added to the solution of compound obtained from step 6 above (1 g, 1.17 mmol) in DCM (10 mL) was stirred at 25° C. for 1 h. The reaction was adjusted to pH=6 with Et₃N, then the solvent was removed under reduced pressure to give a residue. The residue was purified by Pre-HPLC (column: Phenomenex Genimi NX C18 150*40 mm*5 um; mobile phase: [water (0.05% NH3H2O+10 mM NH4HCO3)-ACN]; B %: 1%-30%, 8 min). The desired compound (30 mg, yield: 3.05%) was obtained as a white solid.

MS (ESI) m/z (M+H)⁺: 813.1.

¹H NMR (400 MHz, CD30D) δ 7.45 (s, 1H), 5.76-5.6 (m, 2H), 5.50-5.30 (m, 1H), 5.25-5.15 (m, 1H), 4.78-4.68 (m, 2H), 4.65-4.35 (m, 3H), 4.32-4.06 (m, 4H), 3.94-3.76 (m, 1H), 3.02 (q, J=8.0 Hz, 14H), 2.19-1.90 (m, 12H), 1.24 (t, J=8.0 Hz, 20H).

### Compound A31

Step 1. Preparation of compound (2R,3R,4R,5R)-2-(4-amino-1H-pyrazolo[3,4-d]pyrimidin-1-yl)-5-((benzoyloxy)methyl)tetrahydrofuran-3,4-diyl dibenzoate.

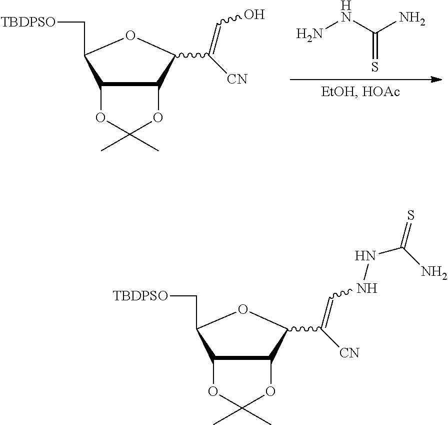

The solution of (E)-2-[(4S,6R,6aS)-6-[[tert-butyl(diphenyl)silyl]oxymethyl]-2,2-dimethyl-3a,4,6,6a-tetrahydrofuro[3,4-d][1,3]dioxol-4-yl]-3-hydroxy-prop-2-enenitrile (6.5 g, 13.55 mmol, 1 eq), aminothiourea (1.85 g, 20.33 mmol, 1.5 eq), AcOH (2.71 g, 45.20 mmol, 2.59 mL, 3.34 eq), H₂O (15 mL) in EtOH (60 mL) was stirred at 80° C. for 2 h. The reaction was concentrated under reduced pressure to remove solvent. The residue was purified by silica gel chromatography (Petroleum ether/Ethyl acetate=20/1, 5/1). The desired (6 g, yield: 80.10%) was obtained as yellow syrups.

Step 2. Preparation of compound 4-[(4S,6R,6aS)-6-[[tert-butyl(diphenyl)silyl]oxymethyl]-2,2-dimethyl-3a,4,6,6a-tetrahydrofuro[3,4-d][1,3]dioxol-4-yl]-5-amino-pyrazole-1-carbothioamide.

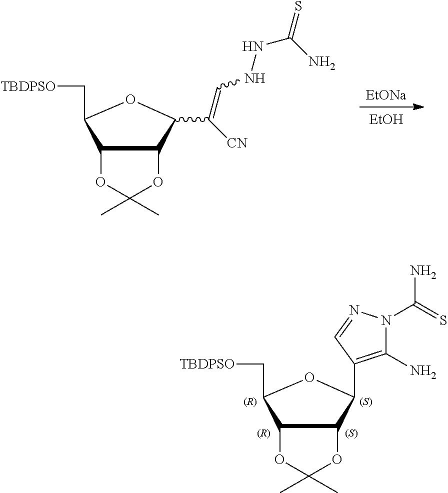

To a solution of compound obtained from step 1 above (4 g, 7.24 mmol) in EtOH (5 mL) was added EtONa (1 M, 7.24 mL) and the reaction was stirred at 25° C. for 0.5 h. The reaction was quenched with AcOH (500 mg) and the mixture was concentrated under reduced. The residue was purified by silica gel chromatography (petroleum ether/Ethyl acetate=20/1, 2/1). The desired (3.5 g, yield: 87.50%) was obtained as yellow syrups.

MS (ESI) m/z (M+H)⁺: 553.2.

Step 3. Preparation of compound 5-amino-4-((3aS,6R,6aR)-6-(hydroxymethyl)-2,2-dimethyltetrahydrofuro[3,4-d][1,3]dioxol-4-yl)-1H-pyrazole-1-carbothioamide.

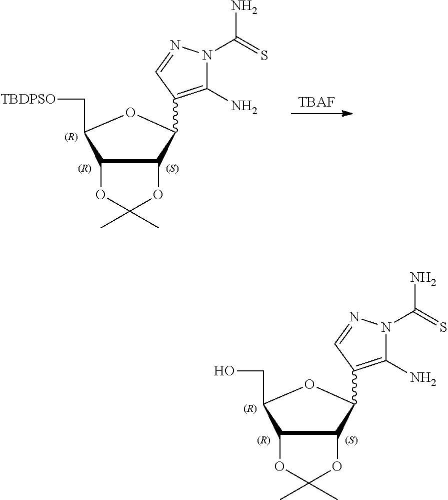

TBAF (1 M, 10.58 mL, 1.5 eq) was added to the solution of compound obtained from step 2 above (3.9 g, 7.06 mmol, 1 eq) in THF (40 mL) and then stirred at 25° C. for 1 h. The reaction was diluted with EtOAc (60 mL), extracted with H₂O (30 mL×3) and brine (30 mL). The organic phase was concentrated to give the residue. The residue was purified by silica gel chromatography (PE: E A=1:0 to 1:2). The desired compound (840 mg, yield: 37.87%) was obtained as yellow oil.

MS (ESI) m/z (M+Na⁺): 336.9.

¹H NMR (400 MHz, CDCl₃) δ 8.57 (s, 1H), 7.31 (d, J=6.0 Hz, 1H), 6.71 (s, 1H), 4.95-4.73 (m, 2H), 4.72-4.53 (m, 1H), 4.29-4.05 (m, 2H), 4.02-3.89 (m, 1H), 3.86-3.75 (m, 1H), 1.65-1.50 (m, 3H), 1.45-1.31 (m, 3H).

Step 4. Preparation of compound ((3aR,4R,6aS)-6-(5-amino-1-carbamothioyl-1H-pyrazol-4-yl)-2,2-dimethyltetrahydrofuro[3,4-d][1,3]dioxol-4-yl)methyl hydrogen phosphonate.

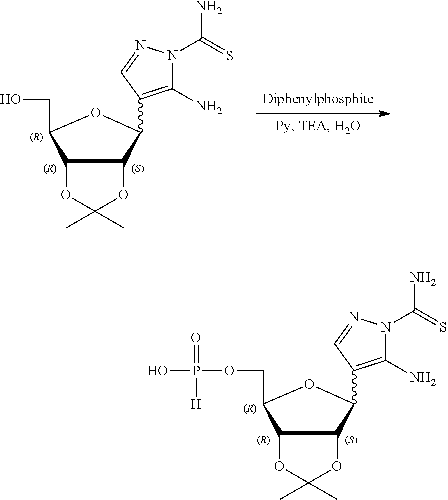

Diphenylphosphite (949.84 mg, 4.06 mmol) was added to the solution of compound obtained from step 3 above (840 mg, 2.67 mmol) in pyridine (8 mL) and stirred at 20° C. for 4 h. Then TEA (1.35 g, 13.36 mmol, 1.86 mL) and H₂O (481.39 mg, 26.72 mmol, 481.39 μL) was added and stirred at 20° C. for 0.5 h. The solvent was removed under reduced pressure to give the crude product. The reaction was diluted with DCM (20 mL) and mixed with silica gel and then purified on silica gel chromatography column (DCM:MeOH=1:0 to 1:1, adding 0.5% Et₃N). The desired product (1.76 g, crude, 2.7Et₃N) was obtained as a brown solid.

MS (ESI) m/z (M+Na⁺): 378.9.

Step 5. Preparation of compound O-(((3aR,4R,6aS)-6-(5-amino-1-carbamothioyl-1H-pyrazol-4-yl)-2,2-dimethyltetrahydrofuro[3,4-d][1,3]dioxol-4-yl)methyl) 0,0-dihydrogen phosphorothioate.

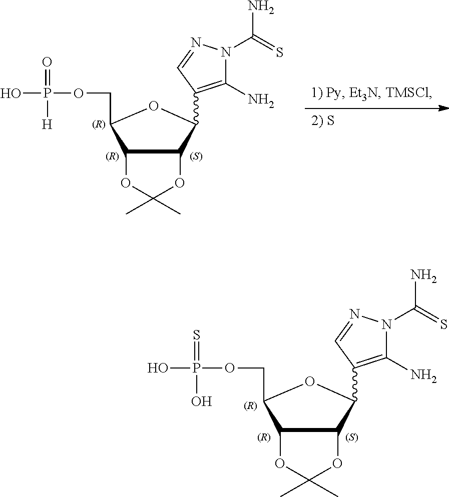

TMSCl (2.35 g, 21.61 mmol, 2.74 mL) was added dropwise to the solution of compound obtained from step 4 above (1.76 g, 2.70 mmol, 2.7Et₃N) in pyridine (8 mL) and Et₃N (8 mL) at 0° C. during 1 h. The reaction was stirred at 0° C. for 1 h, then sulfur (866.16 mg, 27.01 mmol) was added and stirred at 0° C. for 60 min. H₂O (340.65 mg, 18.91 mmol, 340.65 uL, 7 eq) was added to quench the reaction. The reaction was filtered and washed with MeOH (10 mL×3) to remove the solid and the filtrated was concentrated under reduced pressure to give the residue. The residue was purified by silica gel chromatography (DCM: MeOH=1:0 to 1:1, adding 0.5% Et₃N). The desired compound (840 mg, yield: 50.75%, 2Et₃N) was obtained as a yellow solid.

MS (ESI) m/z (M-60+H)⁺: 351.8. (Fragment)

Step 6. Preparation of compound (2S,3S,4S,5S,6S)-2-((S)-2-acetoxy-1-fluoroethyl)-6-(((((((3aR,4R,6aS)-6-(5-amino-1-carbamoyl-1H-pyrazol-4-yl)-2,2-dimethyltetrahydrofuro[3,4-d][1,3]dioxol-4-yl)methoxy)(hydroxy)phosphorothioyl)oxy)(hydroxy)phosphoryl)oxy)tetrahydro-2H-pyran-3,4,5-triyl triacetate.

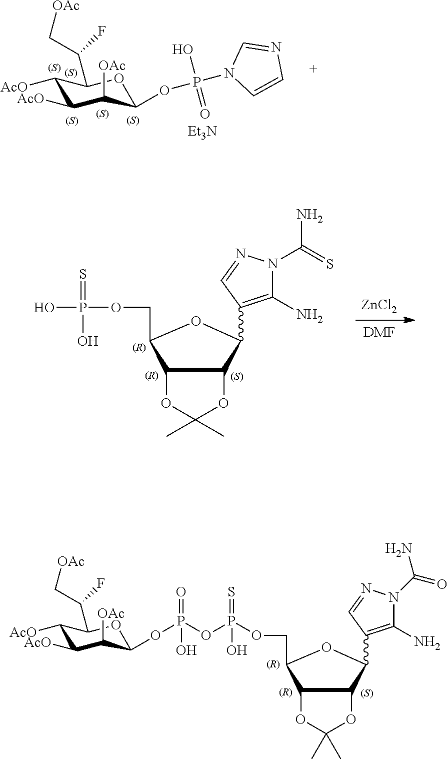

The compound obtained from step 5 above (428.31 mg, 773.56 umol, 2Et₃N) was added to the solution of imidazol-1-yl-[(2S,3R,5S)-3,4,5-triacetoxy-6-[(1S)-2-acetoxy-1-fluoro-ethyl]tetrahydropyran-2-yl]oxy-phosphinic acid (473.07 mg, 773.56 μmol, 1 eq, Et₃N) in DMF (5 mL) under N₂ atmosphere. Then ZnCl₂ (1.58 g, 11.60 mmol, 543.47 μL, 15 eq) was added and the reaction was stirred at 25° C. for 16 h under N₂ atmosphere. The reaction was diluted with DCM (20 mL) and mixed with silica gel and then purified by silica gel chromatography (DCM:MeOH=1:0 to 1:1, adding 0.5% Et₃N). The desired compound (1.5 g, crude) was obtained as a brown solid.

MS (ESI) m/z (M+H)⁺: 852.2.

Step 7. Preparation of compound (2S,3S,4S,5S,6S)-2-((S)-2-acetoxy-1-fluoroethyl)-6-(((((((2R,3S,4R)-5-(5-amino-1-carbamoyl-1H-pyrazol-4-yl)-3,4-dihydroxytetrahydrofuran-2-yl)methoxy)(hydroxy)phosphorothioyl)oxy)(hydroxy)phosphoryl)oxy)tetrahydro-2H-pyran-3,4,5-triyl triacetate

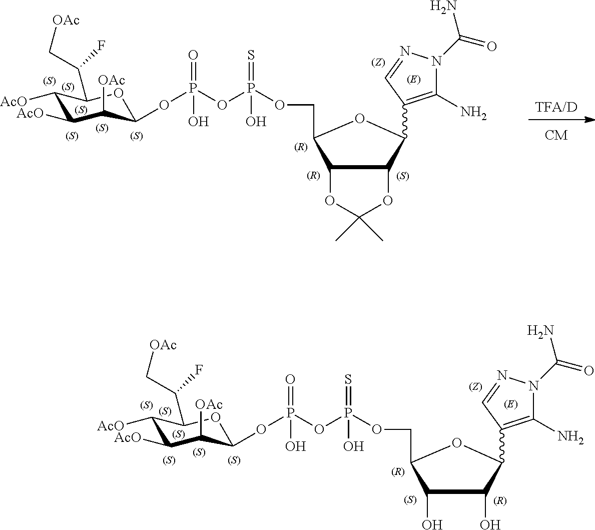

TFA (3.08 g, 27.01 mmol, 2 mL) was added to the mixture of compound obtained from step 6 above (1.3 g, 1.52 mmol) in DCM (5 mL) and stirred at 25° C. for 1.5 h. The reaction was adjusted to pH=6 with Et₃N, then the solvent was removed under reduced pressure to give a residue. The residue was purified by Pre-HPLC

MS (ESI) m/z (M+H)⁺: 812.1.

The invention is further described and exemplified by the following non-limiting examples.

### EXAMPLES

### Example 1. Compound 28 Suppresses HBV Gene Expression and Anti-Sera Production

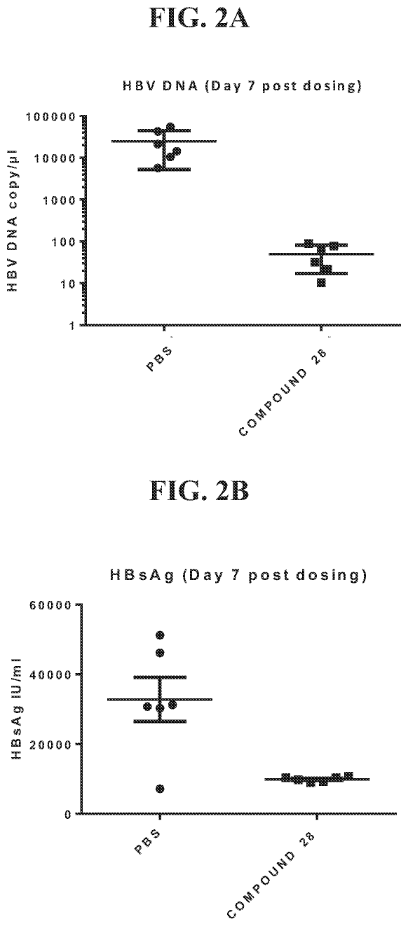

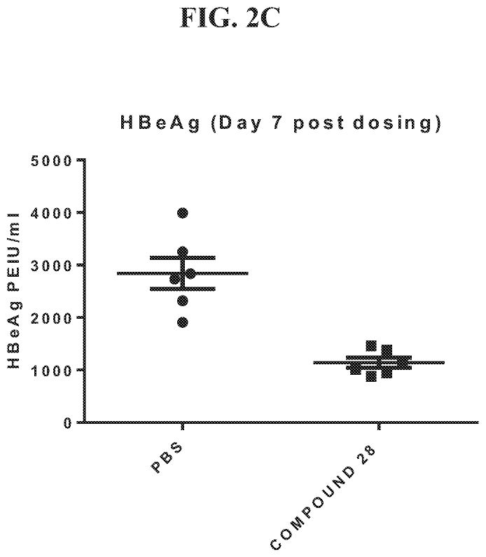

Administration of Compound 28 (0.1 mg/kg, twice weekly “BIW”) led to downregulation of HBV DNA expression and a decrease in serum levels of HBsAg, and HBeAg in the HBV-AAV mouse model. At day 1, male C57BL/6 mice were intravenously injected with AAV8-1.3HBV (1×10¹¹ v/g). After days 28 and 31, each mouse was then intraperitoneally injected with 0.1 mg/kg compound 28 or PBS control. At day 35, mouse serum was collected for HBV DNA qPCR analysis, HBsAg and HBeAg serum analysis by ELISA. Seven days after the first dosing, compound 28-treated mice showed a marked decrease in both HBV DNA expression (FIG. 2A) and serum levels of HBsAg (FIG. 2B) and HBeAg (FIG. 2C).

The structure of Compound 28 is shown below.

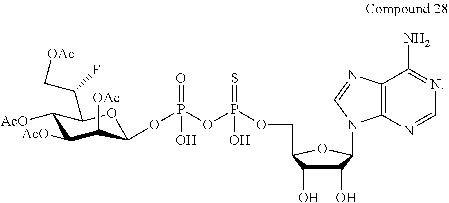

### Example 2. Compound 28 Significantly Increases Production of IgG in Serum Against HBsAg, adw

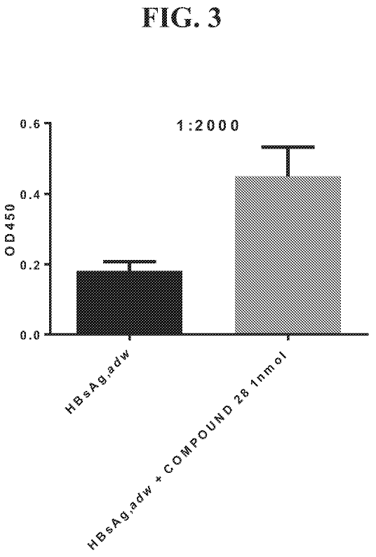

Eight-week-old C57BL/6J mice (females) were intradermally (I.D.) immunized at day 0 with HBsAg, adw (1 mg/dose), or HBsAg, adw at 1 mg/dose combined with compound 28 (1 nmol/dose). Mice were boosted with a second immunization at day 14 and the sera collected at day 35. HBsAg, adw-specific total IgG serum titers (OD₄₅₀) were determined by ELISA. Anti-HBsAg, adw IgG titers were more than 2× higher when HBsAg, adw was combined with 1 nmol/dose of compound 28 than when HBsAg, adw was injected alone (FIG. 3).

### Example 3. Compound 28 Causes a Decrease in the Production of Immune Cells that Contribute to Asthma and COPD

Eight-week-old male C57BL/6 mice were grouped (N=8/group) and were intraperitoneally (I.P.) injected with compound 28 at 0, 4, or 20 nmol/dose on days −1, 0, and 1. In addition, mice were intratracheally (I.Tr.) administrated with 0 or 30 μg/dose of porcine pancreas elastase (PPE) dissolved in 50 μL saline on day 0. Mice were sacrificed on day 2 and the immune cells were collected by bronchoalveolar lavage (BAL). Cells were then subjected to fluorophore-conjugated antibody staining and analyzed by flow cytometry. Data were processed with a FlowJo flow cytometer (Treestar). Immune cells were gated as singlets>live cells>CD11c^{high}SiglecF^{low}MHCII^{high} (dendritic cells), CD11c^{low}CD11b^{high}Ly-6G^{high} (neutrophils), and CD11c^{low}CD11b^{high}Ly-6G^{low}SiglecF^{high} (eosinophils).

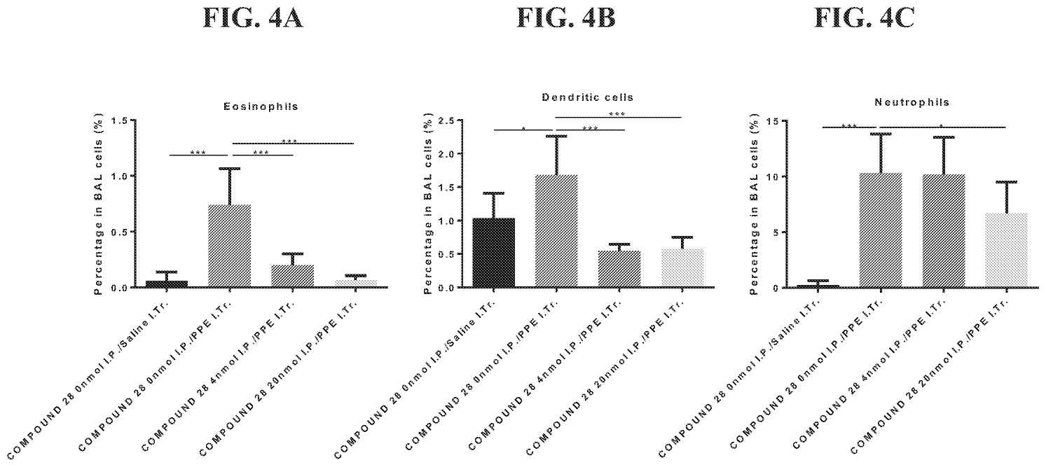

A significant increase in the production of all three types of immune cells, eosinophils (FIG. 4A), dendritic cells (FIG. 4B), and neutrophils (FIG. 4C) was observed at day 2 after PPE administration. The rapid increase in neutrophil production is an early indication of acute elastase-induced emphysema. As shown in FIG. 36, compound 28 at both 4 nmol/dose and 20 nmol/dose significantly suppressed the increase in the production of eosinophils and dendritic cells after PPE administration. The increased production of neutrophils after PPE administration was also suppressed but only at the 20 nmol/dose level. These results suggest that compound 28 may be a potential therapeutic candidate against asthma and COPD, which are characterized by eosinophilic infiltration and neutrophil recruitment in the lung, respectively.

### Example 4. Compound 28 has a Longer Immune Activating Effect Compared to Compound 15

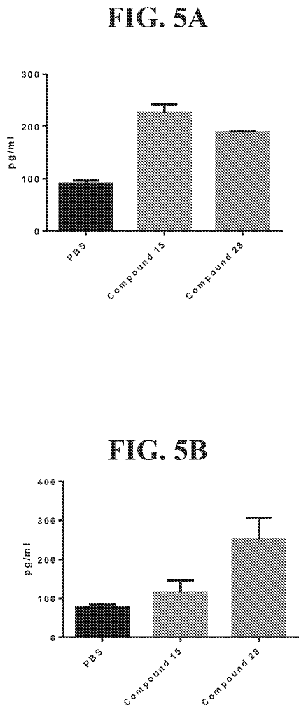

The ability of Compounds 15 and 28 to stimulate ALPK1 activation in vivo was tested by monitoring serum levels of the chemokine RANTES (CCL5), which is elevated upon activation of ALPK1. Briefly, C57/B6 mice (n=3 mice/group) were injected subcutaneously with PBS (negative control) or 10 nmole of Compound 15 or Compound 28. Serum was collected at 24 hours or 48 hours post injection. As shown in FIG. 5A-B, in this assay, Compound 28 showed a longer duration of immune activation, demonstrating an unexpected effect of the fluoro substitution. These results suggest that the fluoro modification may provide the compound with an increased resistance to metabolism, thereby prolonging its in vivo activity.

### Example 5. Compound 28 Derivatives have Increased Cellular Potency

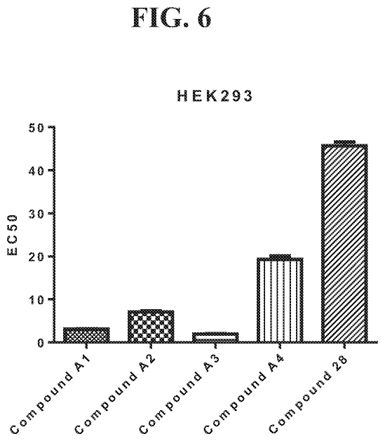

HEK293 cells were treated with H1b-ADP derivatives (A1, A2, A3, and A4) or with Compound 28 for 4 hours followed by analysis of IL-8 secretion using an IL-8 enzyme linked immunoassay (ELISA). IL-8 secretion is an indication of ALPK1 activation. The results are shown in FIG. 6 and Table 2A below. The EC50 of each compound was determined using GraphPad Prism.

TABLE 2A
Activity of Compound 28 and derivatives in FIG. 6.

| Compound | EC50 (nM) |
|---|---|
| 28 | 45.0 |
| A1 | 3.1 |
| A2 | 7.1 |
| A3 | 2.0 |
| A4 | 19.2 |

The results show much lower EC50 values for the derivatives, compared to Compound 28, indicating that the long chain fatty acid of the derivative compounds enhances biological activity, perhaps by facilitating entry of the compound into the cells.

### Example 6. Agonist Activity of Compound 28 Derivatives Using NF-kB Reporter System Also Shows Increased Potency of Derivatives

In the previous example, Example 5, IL-8 secretion was used as an indicator of activated ALPK1 signaling. IL-8 secretion is downstream of NF-kB activation by ALPK1/TIFA/TRAF6, but IL-8 secretion can also be promoted by TIFA/TRAF6 in an ALPK1 independent manner. In this example, NF-kB promoter driven gene expression is used as a more direct indicator of ALPK1 activation. In our studies the EC50 values obtained with the two assays were similar.

NF-kB activation was assayed using HEK293 cells stably expressing an NF-kB reporter construct comprising an NF-kB promoter driving expression of alkaline phosphatase (AP). In this system, activation of NF-kB promoter activity drives the expression of AP which is detected using a chromogenic substrate, para-nitrophenyl phosphate (pNPP).

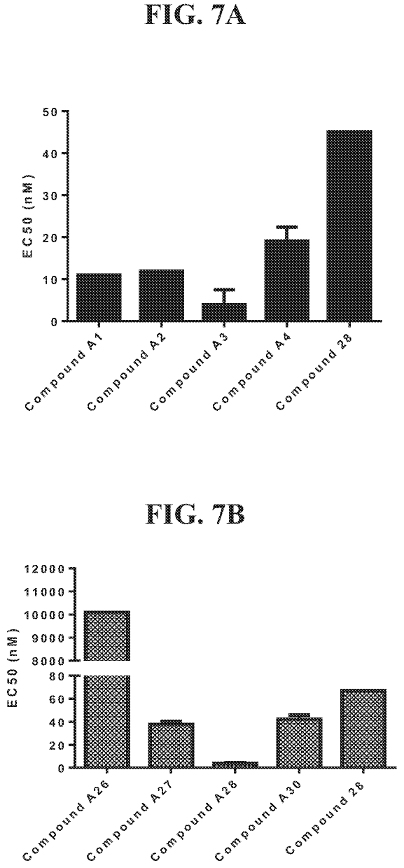

To assess the ALPK1 agonist activity of test compounds, the HEK293 cells were seeded in 96-well plates and allowed to attach overnight. Cells were then exposed to graded concentrations of compounds A1, A2, A3, A4, A18, A26, A27, A28, and A30. Compound 28 was included as a reference. Following treatment, cell culture supernatants were collected and analyzed for alkaline phosphatase activity using the pNPP assay. The results are shown in FIGS. 7A-7B and Table 2B-C below. The EC50 for each compound was determined using GraphPad Prism. Compound A18 did not have activity (data not shown).

TABLE 2B
Activity of Compound 28 and derivatives in FIG. 7A.

| Compound | EC50 (nM) |
|---|---|
| 28 | 45.0 |
| A1 | 11.0 |
| A2 | 11.9 |
| A3 | 3.9 |
| A4 | 19.1 |

TABLE 2C
Activity of Compound 28 and derivatives in FIG. 7B.

| Compound | EC50 (nM) |
|---|---|
| 28 | 67.0 |
| A26 | 10,096 |
| A27 | 37.8 |
| A28 | 3.9 |
| A30 | 42.3 |

The data indicate that the carboside derivatives are more active than the nitroside derivatives. This may be due to increased hydrogen bonding in the case of the carboside derivatives, which may serve to increase ALPK1 agonist activity.

Those skilled in the art will recognize or be able to ascertain using no more than routine experimentation, many equivalents to the specific embodiments of the invention as described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A compound represented by formula (I): 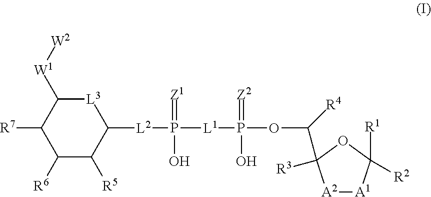
and/or a stereoisomer, tautomer, stable isotope, prodrug or pharmaceutically acceptable salt thereof, wherein:
A¹ and A² are independently selected from O, S and —C(R⁸R⁹)—, wherein
R⁸ and R⁹ are independently selected from H, D, —OH, N₃, —CN, halogen and an optional substituted group selected C1-C4 alkyl, C1-C4 alkoxy, C1-C4 haloalkyl, C1-C4 haloalkoxy, C1-C4 alkanoyloxy, C1-C4 alkenyloxy and aralkyloxy, wherein the optional substituents are 1-3 substituents independently selected from D, halogen, —OH, ═O, C1-C4 alkyl and C1-C4 alkoxy; at least one of A¹ or A² is —C(R⁸R⁹);
wherein R⁸ or R⁹ in A¹ can cyclize with R⁸ or R⁹ in A² to form C3-C6 cycloalkyl and heterocyclyl containing 3 to 9 ring members and having 1-3 heteroatoms selected from N, O and S as ring members, each optionally substituted by 1-3 substituents independently selected from D, halogen, —OH, ═O, C1-C4 alkyl and C1-C4 alkoxy;
L¹ and L² are independently selected from O, CH₂, CHF and CF₂;
L³ is O, S or CH₂;
Z¹ and Z² are independently selected from O and S;
W¹ is —C(R¹⁰R¹¹)—, wherein
R¹⁰ and R¹¹ are independently selected from H, D, —OH, halogen, and optionally substituted groups selected from C1-C4 alkyl, C1-C4 alkoxy, C1-C4 haloalkyl, C1-C4-haloalkoxy, C1-C4 alkenyloxy, aralkyloxy, 1-6 membered oligopeptidyl linked via C-terminal C(O)O— and R¹²CO₂—, wherein
R¹² is selected from C1-C20 alkyl, C1-C20 alkenyl, C1-C20 alkoxy, C1-C20 alkenyloxy, C1-C20 alkylamino, C3-C6 cycloalkyl, heterocyclyl containing 4 to 6 ring members and having 1-3 heteroatoms selected from N, O and S as ring members, C6-C10 aryl, and heteroaryl containing 5 to 10 ring atoms and having 1-3 heteroatoms selected from N, O and S as ring members and 1-6 membered oligopeptidyl linked via N-terminal N;
wherein the optional substituents for R¹⁰ and R¹¹ are 1-3 substituents independently selected from D, halogen, —OH, ═O, C1-C4 alkyl and C1-C4 alkoxy;
wherein when W¹ is —C(R¹⁰R¹¹)— and R¹⁰ is F, and the others are defined as above, W² is H or C1-C3 alkyl optionally substituted with 1-3 substituents independently selected from D, halogen, —OH, ═O, C1-C3 alkoxyl, C1-C3 haloalkyl, C1-C3 haloalkoxyl, C1-C3 alkenyloxyl and R¹²CO₂—, wherein R¹² is C1-C4 alkyl, C1-C4 alkoxy, C1-C4 alkylamino, C3-C6 cycloalkyl, cycloheteroalkyl containing 3 to 6 ring members and having 1-3 heteroatoms selected from N, O and S as ring members, C6-C10 aryl, and heteroaryl containing 5 to 10 ring atoms and having 1-3 heteroatoms selected from N, O and S as ring members;
W² is R¹³-Q¹-W³—, wherein
Q¹ is selected from —O— or —NH—,
W³ is selected from a bond or C1-C3 alkylene groups optionally substituted with 1-3 substituents independently selected from halogen, —OH, C1-C3 alkoxy, C1-C3 haloalkyl, C1-C3 haloalkoxy, C1-C3 alkenyloxy,
R¹³ is 1-6 membered oligopeptidyl linked via C-terminal carbonyl group or
R¹⁴Q²C(O)—, wherein
Q² is a bond, —O— or —NH—,
R¹⁴ is 1-6 membered oligopeptidyl linked via N-terminal N or an optionally substituted group selected from C5-C20 alkyl, C5-C20 alkylenyl and R¹⁵-Q³-Q⁴-Q⁵-, wherein
Q³,Q⁴ and Q⁵ are independently selected from a bond, aryl, heteroaryl containing 5 to 6 ring atoms, C3-C6 cycloalkyl and heterocyclyl containing 4 to 6 ring members and having 1-3 heteroatoms selected from N, O and S as ring members, and at least one of Q³, Q⁴ and Q⁵ is not a bond,
R¹⁵ is an optionally substituted group selected from C1-C18 alky and C1-C18 alkoxy;
wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy;
R¹ is aryl or heteroaryl containing 5 to 10 ring atoms and having 1-4 heteroatoms selected from N, O and S as ring members, wherein R¹ is optionally substituted with 1-3 substituents selected from D, halogen, —OH, ═O, CN, NH₂ and an optionally substituted group selected from C1-C4 alkyl, C1-C4 alkoxy, (R¹⁶R¹⁷)N— and (R¹⁶R¹⁷)NCO—, wherein
R¹⁶ and R¹⁷ are independently selected from H and an optionally substituted group selected from C1-C4 alkyl, C3-C6 cycloalkyl, heterocyclyl containing 4 to 6 ring members and having 1-3 heteroatoms selected from N, O and S as ring members, aryl, arylalkyl, heteroaryl containing 5 to 10 ring atoms and having 1-3 heteroatoms selected from N, O and S as ring members; and heteroarylalkyl containing 5 to 10 ring atoms and having 1-3 heteroatoms selected from N, O and S as ring members; wherein the optional substituents are 1-3 substituents independently selected from from D, halogen, —OH, ═O, CN, NH₂ and an C1-C4 alkyl, C1-C4 alkoxy, C1-C3 haloalkyl, C1-C3 haloalkoxy and C3-C6 cycloalkyl;
R², R³ and R⁴ are independently selected from H, D, halogen, C1-C4 alkyl and C1-C4 haloalkyl;
R⁵, R⁶ and R⁷ are independently selected from H, —OH, halogen, and R¹²CO₂—, and at least two of R⁵, R⁶ and R⁷ are OH or R¹²CO₂—, wherein R¹² is selected from C1-C4 alkyl, C1-C4 alkoxy, C1-C4 alkenyloxy, C1-C4 alkylamino, C3-C6 cycloalkyl, heterocyclyl containing 4 to 6 ring members and having 1-3 heteroatoms selected from N, O and S as ring members, C6-C10 aryl, and heteroaryl containing 5 to 10 ring atoms and having 1-3 heteroatoms selected from N, O and S as ring members;
wherein any two of the adjacent groups of R⁵, R⁶ and R⁷ can cyclize to form heterocyclyl containing 5 to 9 ring members and having 1-3 heteroatoms selected from N, O and S as ring members, each optionally substituted by 1-3 substituents independently selected from D, halogen, —OH, ═O, C1-C4 alkyl and C1-C4 alkoxy.

2. The compound according to claim 1, which is a compound of Formula IA, and/or a stereoisomer, a stable isotope, prodrug or a pharmaceutically acceptable salt thereof 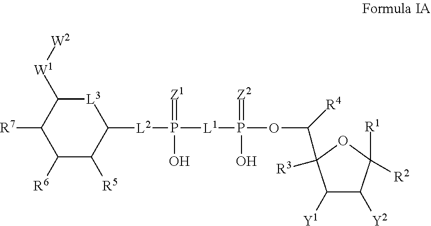
wherein:
Y¹ and Y² are independently selected from H, D, —OH, N₃, —CN, halogen and optionally substituted groups selected from C1-C4 alkyl, C1-C4 alkoxy, C1-C4 haloalkyl, C1-C4 haloalkoxy, C1-C4 alkanoyloxy, C1-C4 alkenyloxy and aralkyloxy;
wherein the optional substituents are 1-3 substituents independently selected from D, halogen, —OH, —O, C1-C4 alkyl and C1-C4 alkoxy; and
R¹-R⁷, L¹-L³, Z¹, Z², W¹ and W² are defined as in claim 1.

3. The compound according to claim 2, wherein
Y¹ and Y² are independently selected from H, D, —OH, halogen, C1-C4 alkyl, C1-C4 alkoxy, C1-C4 haloalkyl, C1-C4 haloalkoxy, C1-C4 alkanoyloxy and C1-C4 alkenyloxy;
R¹-R⁷, L¹-L³, Z¹, Z², W¹ and W² are defined as in claim 1.

4. The compound according to claim 2, wherein
Y¹ and Y² are independently selected from —OH, halogen, C1-C4 alkyl and C1-C4 alkanoyloxy;
R¹-R⁷, L¹-L³, Z¹, Z², W¹ and W² are defined as in claim 1.

5. The compound according to claim 1, which is a compound of Formula IB, and/or a stereoisomer, a stable isotope, prodrug or a pharmaceutically acceptable salt thereof: 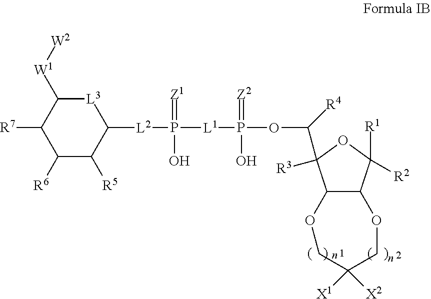
wherein:
n¹ and n² are each an integer independently selected from the group consisting of 0-2;
X¹ and X² are independently selected from H, D, —OH, N₃, —CN, halogen and optionally substituted groups selected from C1-C4 alkyl, C1-C4 alkoxy, C1-C4 haloalkyl, C1-C4 haloalkoxy, C1-C4 alkanoyloxy, C1-C4 alkenyloxy and aralkyloxy;
wherein the optional substituents are 1-3 substituents independently selected from D, halogen, —OH, ═O, C1-C4 alkyl and C1-C4 alkoxy;
R¹-R⁷, L¹-L³, Z¹, Z², W¹ and W² are defined as in claim 1.

6. The compound according to claim 5, wherein n¹ and n² are each 0.

7. The compound according to claim 5, wherein
X¹ and X² are independently selected from H, D, and C1-C4 alkyl;
R¹-R⁷, L¹-L³, Z¹, Z², W¹ and W² are defined as in claim 1.

8. The compound according to claim 1, which is a compound of Formula IC, and/or a stereoisomer, a stable isotope, prodrug or a pharmaceutically acceptable salt thereof: 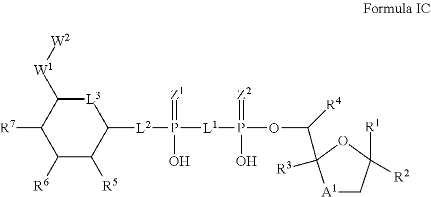
wherein:
A¹ is —C(R¹⁰R¹¹)—, O or S;
R¹-R¹¹, L¹-L³, Z¹, Z², W¹ and W² are defined as in Formula I.

9. The compound according to claim 1, wherein R², R³, and R⁴ are each H.

10. The compound according to claim 1, wherein R⁵, R⁶, and R⁷ are each independently selected from the group consisting of —OH, and C1-C4 alkanoyloxy.

11. The compound according to claim 1, wherein L³ is O.

12. The compound according to claim 1, wherein L² is O.

13. The compound according to claim 1, wherein L¹ is O or S.

14. The compound according to claim 1, wherein W¹ is —C(R¹⁰R¹¹)—, wherein R¹⁰ and R¹¹ are independently selected from H, D, —OH, halogen, C1-C4 alkyl, C1-C4 alkoxy, C1-C4 haloalkyl, C1-C4-haloalkoxy, C1-C4 alkanoyloxy, C1-C4 alkenyloxy and R¹²CO₂—, wherein R¹² is selected from C1-C20 alkyl, C1-C20 alkoxy, C1-C20 alkanoyloxy and C1-C20 alkenyloxy.

15. The compound according to claim 1, wherein W¹ is —C(R¹⁰R¹¹)—, wherein R¹⁰ and R¹¹ are independently selected from H, D, —OH, halogen and C1-C20 alkanoyloxy.

16. The compound according to claim 1, wherein W² in formulas I, IA, IB, and IC is R¹³-Q¹-W³—, wherein Q¹ is —O—; W³ is a C1 alkylene group and R¹³ wherein R¹³ is 1-6 membered oligopeptidyl linked via C-terminal carbonyl group;

17. The compound according to claim 1, wherein W² in formulas I, IA, IB, and IC is R¹³-Q¹-W³—, wherein Q¹ is —O—; W³ a C1 alkylene group and R¹³ is R¹⁴Q²C(O)—; wherein R¹⁴ is 1-6 membered oligopeptidyl linked via N-terminal group, Q² is a bond;

18. The compound according to claim 1, wherein W² in formulas I, IA, IB, and IC is R¹³-Q¹-W³—, wherein Q¹ is —O—; W³ is a C1 alkylene group and R¹³ is R¹⁴Q²C(O)—; wherein R¹⁴ is an optionally substituted group selected from C5-C20 alkyl and C5-C20 alkylenyl;

19. The compound according to claim 1, wherein W² in formulas I, IA, IB, and IC is R¹³-Q¹-W³—, wherein
Q¹ is —O—,
W³ is a C1 alkylene group, and
R¹³ is R¹⁴Q²C(O)—, wherein R¹⁴ is an optionally substituted group selected from R¹⁵-Q³-Q⁴-Q⁵-; Q³,Q⁴ and Q⁵ are independently selected from a bond, aryl, heteroaryl containing 5 to 6 ring atoms, C3-C6 cycloalkyl and heterocyclyl containing 4 to 6 ring members and having 1-3 heteroatoms selected from N, O and S as ring members, and at least one of Q³, Q⁴ and Q⁵ is not a bond; and R¹⁵ is an optionally substituted group selected from C1-C18 alky and C1-C18 alkoxy;
wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy.

20. The compound according to claim 1, wherein W² in formulas I, IA, IB, and IC is R¹³-Q^{L}-W³—, wherein
Q^{L} is —O—,
W³ is a C1 alkylene group and
R¹³ is R¹⁴Q²C(O)—, wherein
R¹⁴ is an optionally substituted group selected from R¹⁵-Q³-Q⁴-Q⁵-; Q³,Q⁴ and Q⁵ are independently selected from a bond, aryl, and at least one of Q³, Q⁴ and Q⁵ is not a bond; R¹⁵ is an optionally substituted group selected from C1-C18 alky and C1-C18 alkoxy,
wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy.

21. The compound according to claim 1, wherein W² in formulas I, IA, IB, and IC is R¹³-Q^{L}-W³—, wherein Q¹ is —O—; W³ is a C1 alkylene group and R¹³ is R¹⁴Q²C(O)—; wherein R¹⁴ is is an optionally substituted group selected from C5-C20 alkyl group and Q² is a bond.

22. The compound according to claim 1, wherein R¹ is selected from

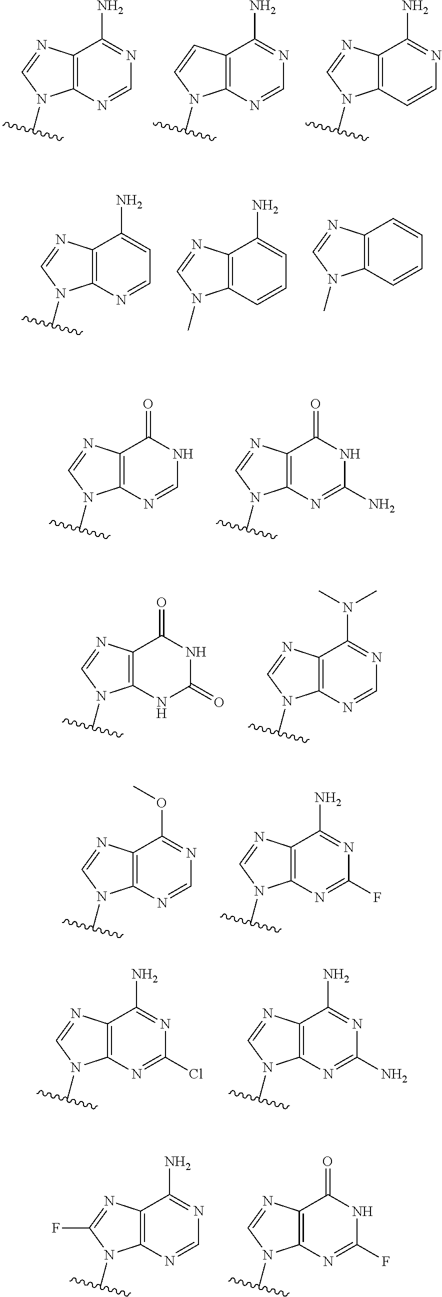

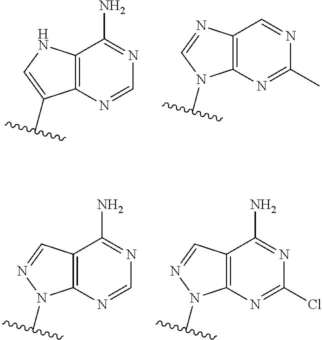 [SMILES: *c1c[nH]c2c(N)ncnc12 ; *n1cnc2cnc(C)nc21 ; *n1ncc2c(N)ncnc21 ; *n1ncc2c(N)nc(Cl)nc21]

23. The compound according to claim 1, wherein R¹ is selected from

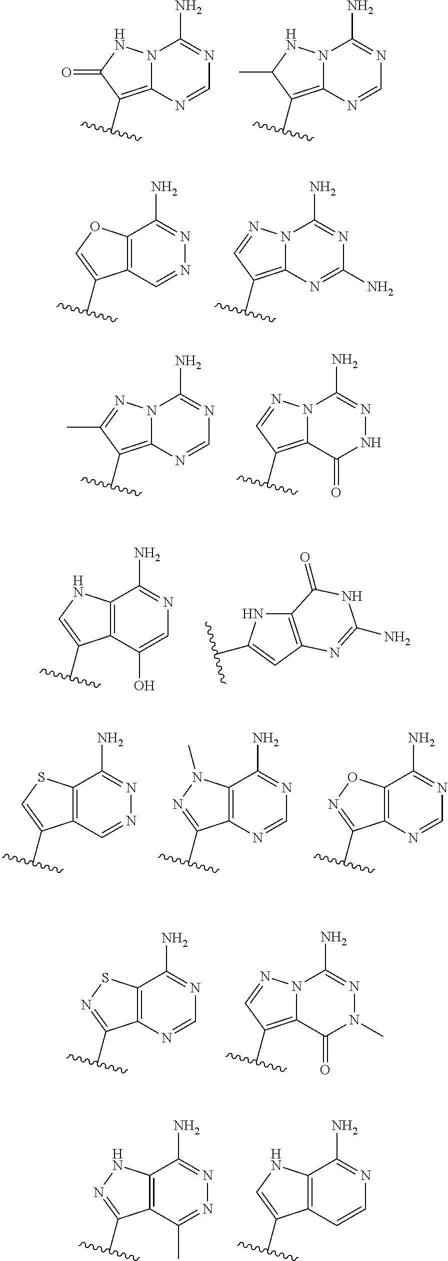

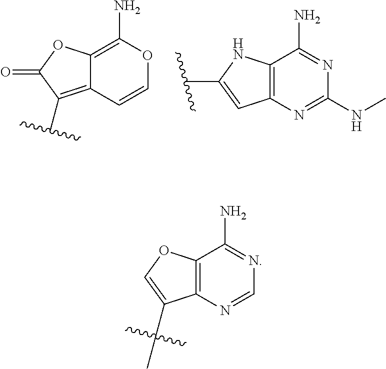

24. The compound according to claim 1, wherein R¹ is selected from

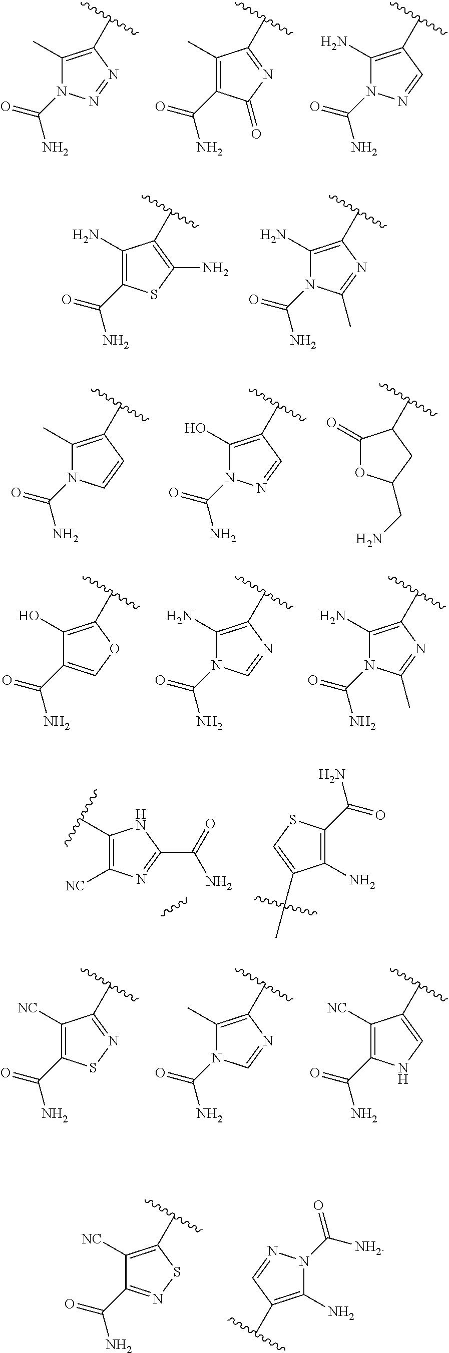

25. The compound according to claim 1, wherein R¹ is selected from 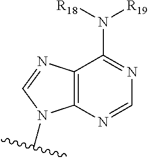
R¹⁸ and R¹⁹ are independently selected from H and an optionally substituted group selected from heterocyclyl C6-C10 arylalkyl, heteroarylalkyl containing 5 to 10 ring atoms and having 1-3 heteroatoms selected from N, O and S as ring members, R²⁰CO— and R²¹S(O)₂—; wherein the substitutents for 1-3 substituents independently selected from halogen, OH, ═O, CN, NH₂, C1-C3 alkyl, C1-C3 haloalkyl, C1-C3 alkyloxy, C1-C3 haloalkyloxy, C3-C6 cycloalkyl, C3-C6 cycloalkyloxy, heterocyclyl containing 4 to 6 ring members and having 1-3 heteroatoms selected from N, O and S as ring members, C6-C10 aryl, C6-C10 haloaryl, heteroaryl containing 5 to 10 ring atoms and having 1-3 heteroatoms selected from N, O and S as ring members, and haloheteroaryl containing 5 to 10 ring atoms and having 1-3 heteroatoms selected from N, O and S as ring members; R²⁰ and R²¹ are C1-C20 alkyl, C1-C20 alkenyl, heterocyclyl containing 4 to 6 ring members and having 1-3 heteroatoms selected from N, O and S as ring members, C6-C10 aryl, C6-C10 haloaryl, heteroaryl containing 5 to 10 ring atoms and having 1-3 heteroatoms selected from N, O and S as ring members, and haloheteroaryl containing 5 to 10 ring atoms and having 1-3 heteroatoms selected from N, O and S as ring members.

26. The compound according to claim 1, wherein R¹ is selected from 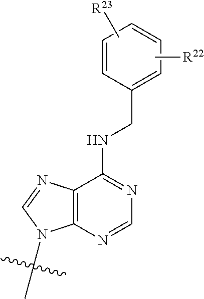
wherein R²² and R²³ are independently selected from H, D halogen, C1-C4 alkyl, C1-C4 alkyloxy, C1-C4 haloalkyl, C1-C4 haloalkyloxy, C1-C4-cycloalkyl and C1-C4-cycloalkyloxy.

27. The compound according to claim 1, which is a compound of Formula ID, and/or a stereoisomer, a stable isotope, prodrug or a pharmaceutically acceptable salt thereof: 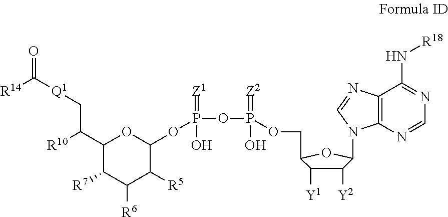
wherein R⁵-R⁷, R¹⁰, R¹⁴, R¹⁸, Q¹, Y1, Y², Z¹ and Z² are as defined above.

28. The compound according to claim 1, which is a compound of Formula IE, and/or a stereoisomer, a stable isotope, prodrug or a pharmaceutically acceptable salt thereof: 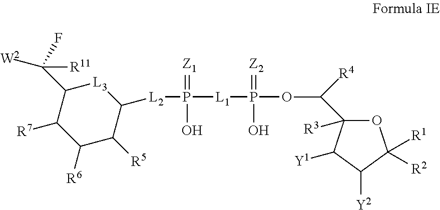
wherein:
Z¹ and Z² are independently selected from O and S wherein at least one of Z¹ and Z² is S;
W² is H or C1-C3 alkyl optionally substituted with 1-3 substituents independently selected from D, halogen, —OH, ═O, C1-C3 alkoxyl, C1-C3 haloalkyl, C1-C3 haloalkoxyl, C1-C3 alkenyloxyl and R¹²CO₂—, wherein R¹² is C1-C4 alkyl, C1-C4 alkoxy, C1-C4 alkylamino, C5-C20 alkyl, C5-C20 alkenyl, C5-C20 alkoxy, C5-C20 alkenyloxy, C5-C20 alkylamino, C3-C6 cycloalkyl, cycloheteroalkyl containing 3 to 6 ring members and having 1-3 heteroatoms selected from N, O and S as ring members, C6-C10 aryl, and heteroaryl containing 5 to 10 ring atoms and having 1-3 heteroatoms selected from N, O and S as ring members;
R¹-R⁷, R¹¹, L¹-L³, Y¹Y2, and W² are defined above.

29. The compound according to claim 28, Z² is S and Z¹ is O.

30. The compound according to claim 28, Z² is S and Z¹ is S.

31. The compound according to claim 28, R², R³, and R⁴ are each H.

32. The compound according to claim 28, R⁵, R⁶, and R⁷ are each independently selected from the group consisting of —OH, and C1-C4 alkanoyloxyl.

33. The compound according to claim 28, L³ is O.

34. The compound according to claim 28, L² is O.

35. The compound according to claim 28, L¹ is O.

36. The compound according to claim 28, R¹¹ is selected from H, D, —OH, halogen, C1-C4 alkyl, C1-C4 alkoxyl, C1-C4 haloalkyl, C1-C4-haloalkoxyl, C1-C4 alkanoyloxyl, C1-C4 alkenyloxyl and R¹²CO₂—, wherein R¹² is selected from C1-C4 alkyl, C1-C4 alkoxyl, C1-C4 alkanoyloxyl and C1-C4 alkenyloxyl.

37. The compound according to claim 28, R¹¹ is selected from H, D, —OH, and halogen.

38. The compound according to claim 28, R¹¹ is H.

39. The compound according to claim 28, W² is C1-C3 alkyl optionally substituted with 1-3 substituents independently selected from D, halogen, —OH, ═O and C1-C3 alkoxyl, C1-C3 haloalkyl, C1-C3 haloalkoxyl, C1-C3 alkenyloxyl and R¹²CO₂—, wherein R¹² is C1-C alkyl, C1-C4 alkoxy and C1-C4 alkylamino.

40. The compound according to claim 28, W² is C1-C3 alkyl optionally substituted with 1-3 substituents independently selected from D, halogen, —OH and R¹²CO₂—, wherein R¹² is C1-C3 alkyl.

41. The compound according to claim 28, W² is C1 alkyl optionally substituted with 1 substituent selected from —OH and R¹²CO₂—, wherein R¹² is C1-C3 alkyl.

42. The compound according to claim 28, R¹ in formulas IE is selected from

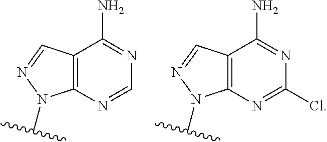 [SMILES: *n1ncc2c(N)ncnc21 ; *n1ncc2c(N)nc(Cl)nc21]

43. The compound according to claim 28, when W² is R¹CO₂—, wherein R¹² is selected from C5-C20 alkyl, C5-C20 alkenyl, C5-C20 alkoxy, C5-C20 alkenyloxy, C5-C20 alkylamino, and R¹ in formulas IE is selected from

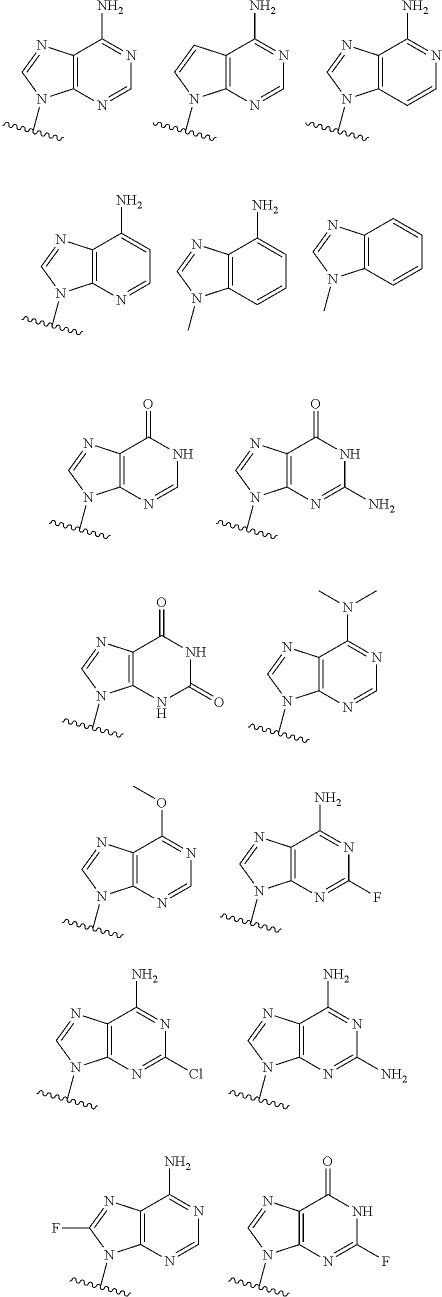

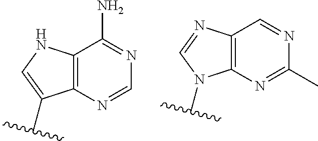 [SMILES: *c1c[nH]c2c(N)ncnc12 ; *n1cnc2cnc(C)nc21]

44. The compound according to claim 28, R¹ in formulas IE is selected from

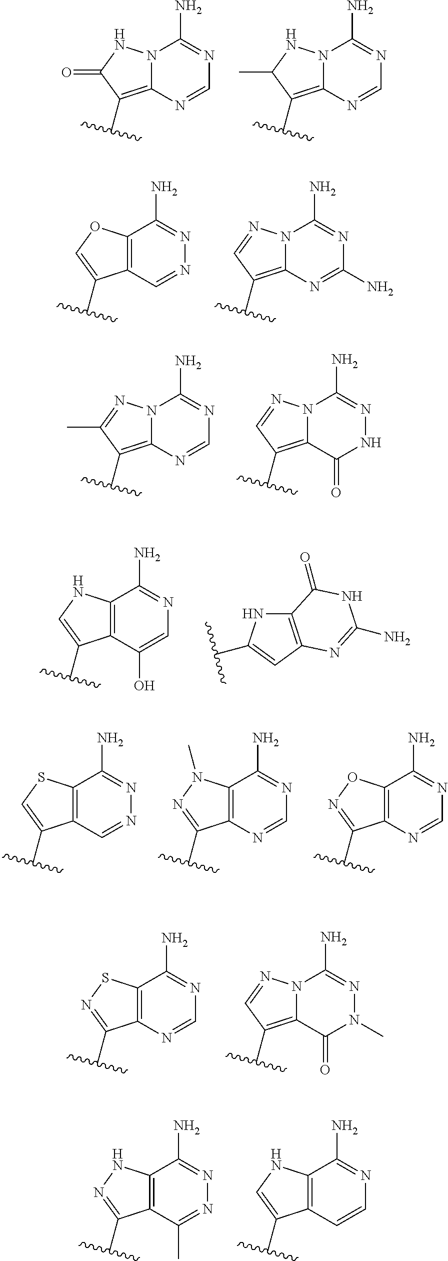

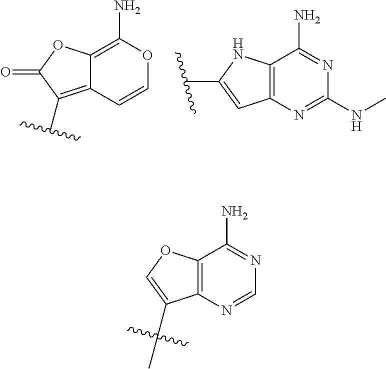

45. The compound according to claim 28, R¹ in formulas IE is selected from

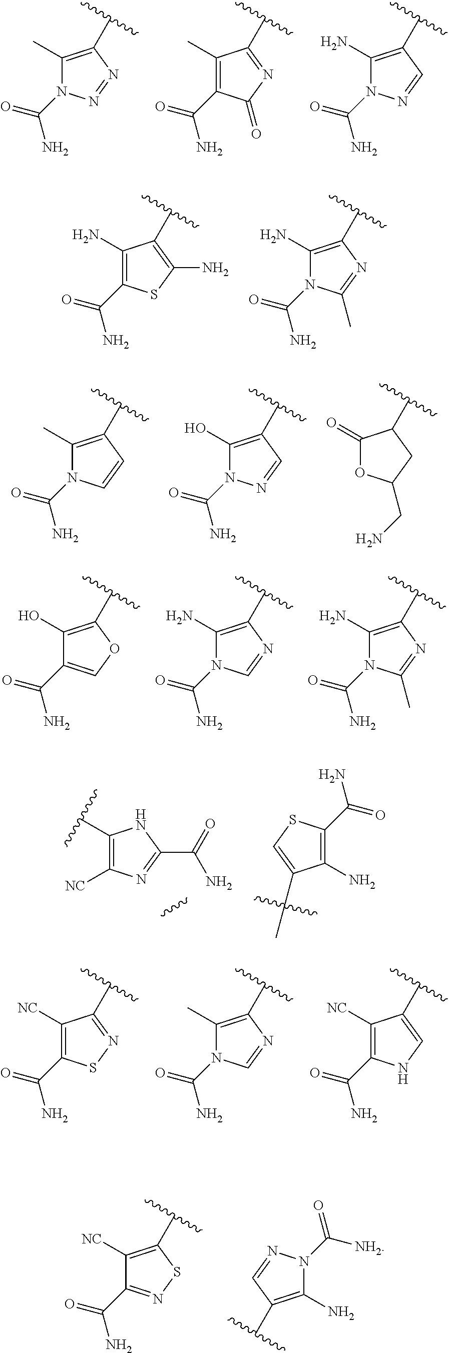

46. The compound according to claim 28, R¹ in formulas IE is selected from 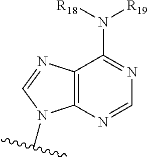
R¹⁸ and R¹⁹ are independently selected from H and an optionally substituted group (“OSG”) selected from heterocyclyl C6-C10 arylalkyl, heteroarylalkyl containing 5 to 10 ring atoms and having 1-3 heteroatoms selected from N, O and S as ring members, R²⁰CO— and R²¹S(O)₂—;
wherein the substituents for the optionally substituted group (“OSG”) are 1-3 substituents independently selected from halogen, OH, ═O, CN, NH₂, C1-C3 alkyl, C1-C3 haloalkyl, C1-C3 alkyloxy, C1-C3 haloalkyloxy, C3-C6 cycloalkyl, C3-C6 cycloalkyloxy, heterocyclyl containing 4 to 6 ring members and having 1-3 heteroatoms selected from N, O and S as ring members, C6-C10 aryl, C6-C10 haloaryl, heteroaryl containing 5 to 10 ring atoms and having 1-3 heteroatoms selected from N, O and S as ring members, and haloheteroaryl containing 5 to 10 ring atoms and having 1-3 heteroatoms selected from N, O and S as ring members; and
R²⁰ and R²¹ are C1-C20 alkyl, C1-C20 alkenyl, heterocyclyl containing 4 to 6 ring members and having 1-3 heteroatoms selected from N, O and S as ring members, C6-C10 aryl, C6-C10 haloaryl, heteroaryl containing 5 to 10 ring atoms and having 1-3 heteroatoms selected from N, O and S as ring members, and haloheteroaryl containing 5 to 10 ring atoms and having 1-3 heteroatoms selected from N, O and S as ring members.

47. The compound according to any one of claim 28, wherein R¹ in formulas IE is selected from 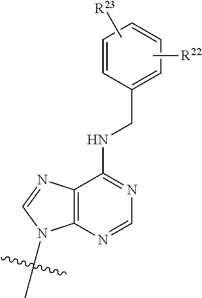
wherein R²² and R²³ are independently selected from H, D halogen, C1-C4 alkyl, C1-C4 alkyloxy, C1-C4 haloalkyl, C1-C4 haloalkyloxy, C1-C4-cycloalkyl and C1-C4-cycloalkyloxy.

48. The compound according to any one of claim 28, when W² is R¹²CO₂—, wherein R¹² is selected from C5-C20 alkyl, C5-C20 alkenyl, C5-C20 alkoxy, C5-C20 alkenyloxy, C5-C20 alkylamino, wherein R¹ in formulas IE is

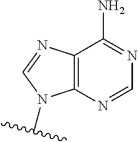 [SMILES: *n1cnc2c(N)ncnc21]

49. The compound according to claim 28, wherein Y¹ and Y² are independently selected from H, D, —OH, halogen, C1-C4 alkyl, C1-C4 alkoxyl, C1-C4 haloalkyl, C1-C4 haloalkoxyl, C1-C4 alkanoyloxyl and C1-C4 alkenyloxyl.

50. The compound according to claim 28, wherein Y¹ and Y² are independently selected from —OH, halogen, C1-C4 alkyl and C1-C4 alkanoyloxyl.

51. The compound according to claim 28, wherein Y¹ and Y² are each —OH.

52. The compound according to claim 28, wherein the compound of formula IE is selected from

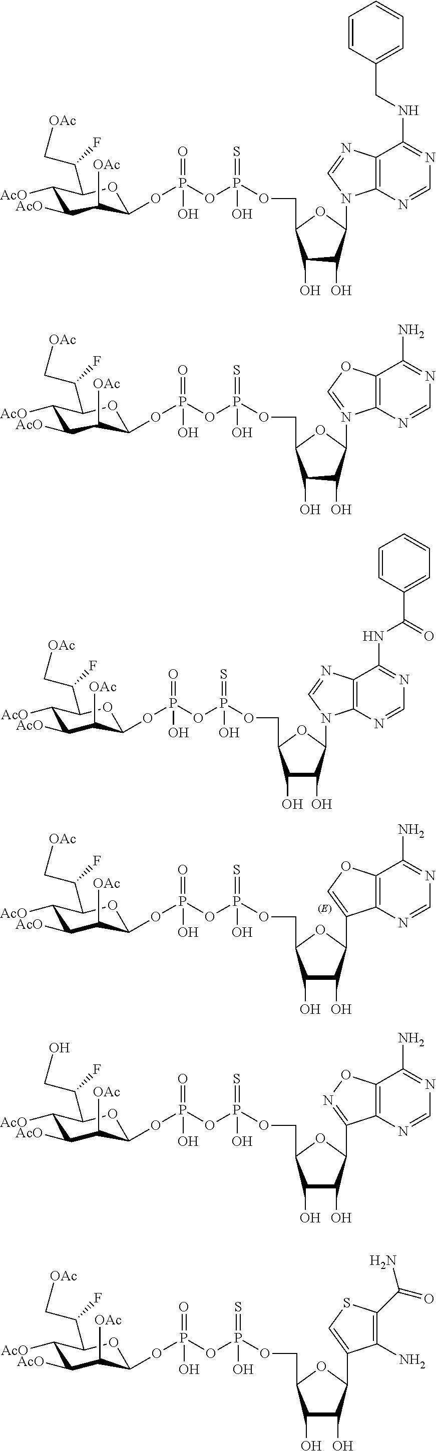

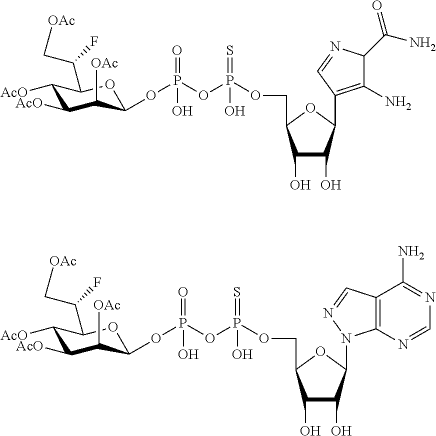

53. The compound according to claim 1, wherein the compound of Formula I is selected from 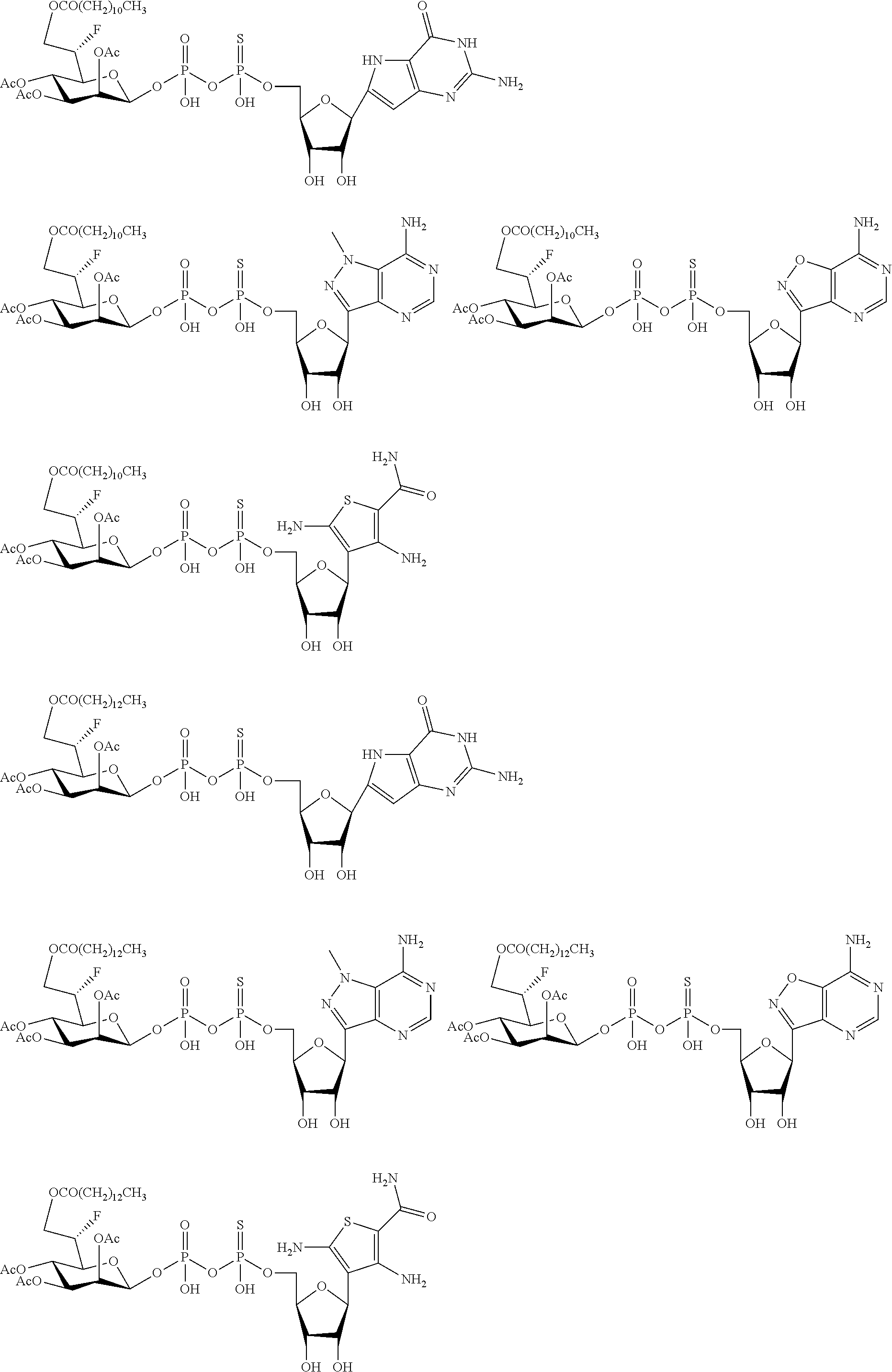 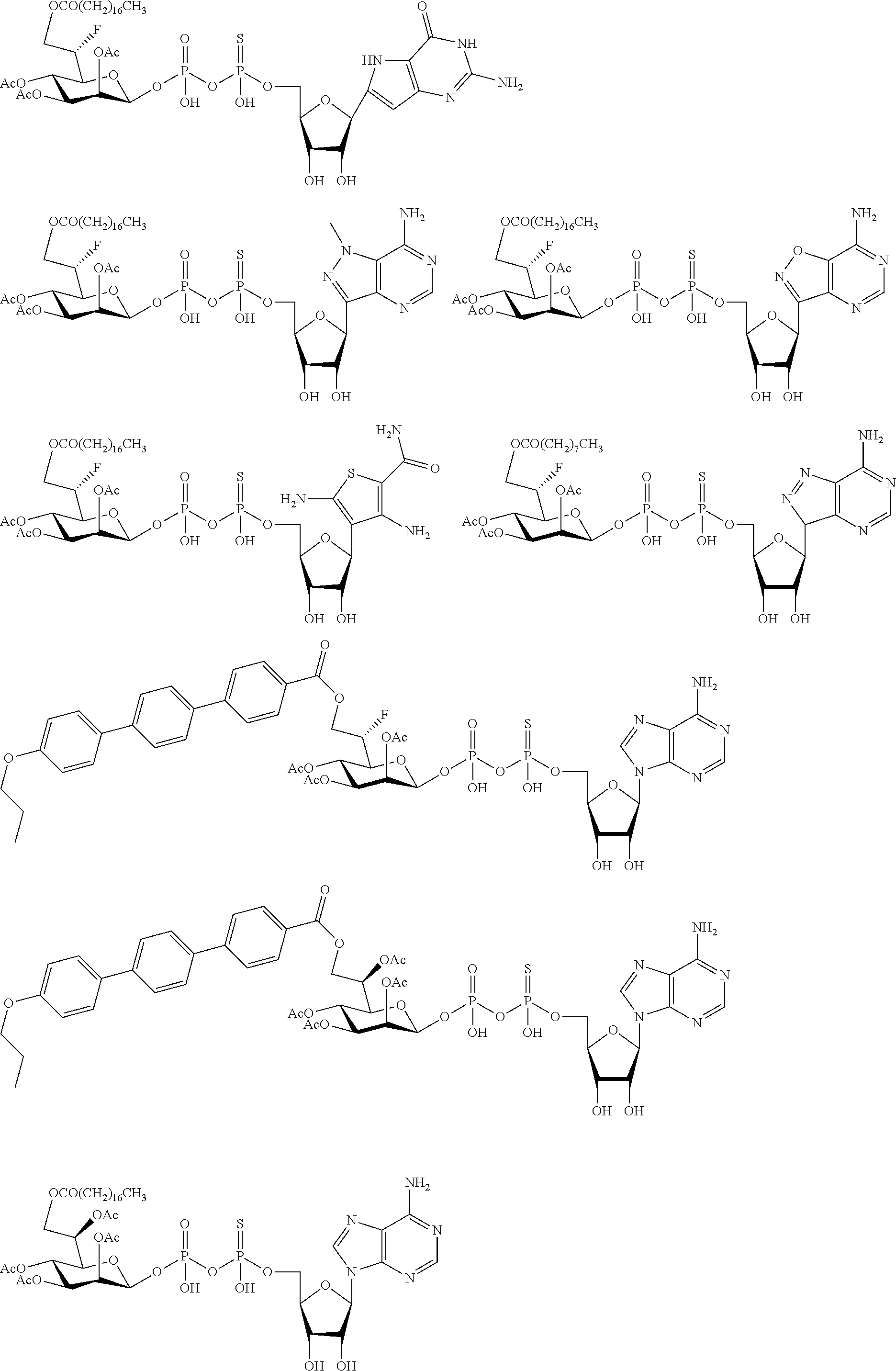 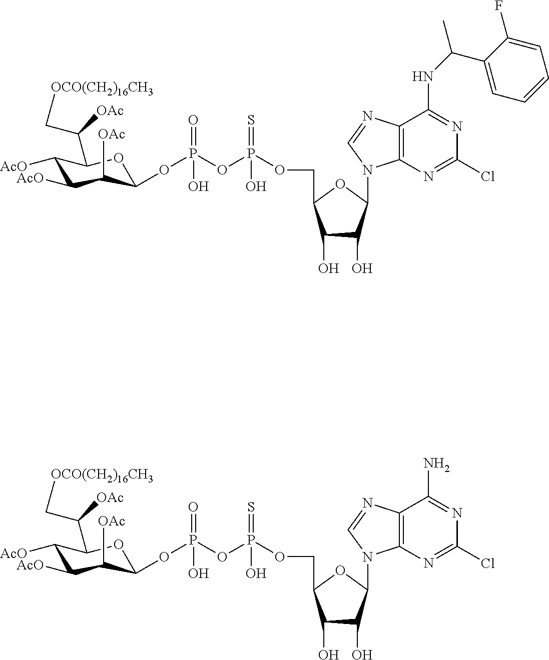
and/or a stereoisomer, a stable isotope, prodrug or a pharmaceutically acceptable salt thereof.

54. A pharmaceutical composition comprising a compound of claim 1 and a pharmaceutically acceptable carrier.

55. A method for activating ALPK1, the method comprising administering an effective amount of a compound or a pharmaceutically acceptable salt of claim 1.

56. A method for modulating an immune response in a subject in need of such treatment, the method comprising administering to the subject an effective amount of a compound or a pharmaceutically acceptable salt of claim 1.

57. A method for treating cancer in a subject in need of such treatment, the method comprising administering to the subject an effective amount of a compound or a pharmaceutically acceptable salt of claim 1.

58. A method for potentiating an immune response to a target antigen in a subject, the method comprising administering to the subject an effective amount of a compound or a pharmaceutically acceptable salt of claim 1.

59. A method for treating a disease or disorder amendable to treatment by activation of NFkB, p38, and JNK cell signaling pathways in cells of a subject, the method comprising administering to the subject an effective amount of a compound or a pharmaceutically acceptable salt of claim 1.

60. A method for treating or preventing a disease or disorder caused by an infectious agent selected from a bacteria, virus, or parasite in a subject in need thereof, the comprising administering to the subject an effective amount of a compound or a pharmaceutically acceptable salt of claim 1.

61. The method of claim 56, wherein modulating an immune response is selected from activation of innate immunity and activation of adaptive immunity.

62. The method of claim 57, wherein the cancer is selected from soft tissue sarcoma, breast cancer, head and neck cancer, melanoma, cervical cancer, bladder cancer, hematologic malignancy, glioblastoma, pancreatic cancer, prostate cancer, colon cancer, breast cancer, renal cancer, lung cancer, merkel cell carcinoma, small intestine cancer, thyroid cancer, acute myelogenous leukemia (AML), acute lymphocytic leukemia (ALL), chronic lymphocytic leukemia (CLL), chronic myelogenous leukemia (CML), gastric cancer, gastrointestinal stromal tumors, non-Hodgkins lymphoma, Hodgkins lymphoma, liver cancer, leukemia, lymphoma, T-cell lymphoma, brain cancer, and multiple myeloma.

63. The method of claim 58, wherein the target antigen is an antigen of an infectious agent selected from the group consisting of adenovirus, Coxsackie B virus, cytomegalovirus, eastern equine encephalitis virus, ebola virus, enterovirus 71, Epstein-Barr virus, Haemophilus influenzae type b (Hib), hepatitis C virus (HCV), herpes virus, human immunodeficiency virus (HIV), human papillomavirus (HPV), hookworm, Marburg virus, norovirus, respiratory syncytial virus (RSV), rotavirus, Salmonella typhi, Staphylococcus aureus, Streptococcus pyogenes, varicella, West Nile virus, Yersinia pestis, and Zika virus.

64. The method of claim 58, wherein the compound acts as a vaccine adjuvant for a vaccine in the treatment or prevention of anthrax, caries, Chagas disease, dengue, diphtheria, ehrlichiosis, hepatitis A or B, herpes, seasonal influenza, Japanese encephalitis, leprosy, lyme disease, malaria, measles, mumps, meningococcal disease, including meningitis and septicemia, Onchocerciasis river blindness, pertussis (whooping cough), pneumococcal disease, polio, rabies, rubella, schistosomiasis, severe acute respiratory syndrome (SARS), shingles, smallpox, syphilis, tetanus, tuberculosis, tularemia, tick-borne encephalitis virus, typhoid fever, trypanosomiasis, yellow fever, or visceral leishmaniasis.

65. The method of claim 59, wherein the disease or disorder is selected from tuberculosis, meningitis, pneumonia, ulcer, sepsis, rhinitis, asthma, allergy, COPD, inflammatory bowel disease, arthritis, obesity, radiation-induced inflammation, psoriasis, atopic dermatitis, non-alcoholic steatohepatitis (NASH), Alzheimer's disease, systemic lupus, erythematosus (SLE), autoimmune thyroiditis (Grave's disease), multiple sclerosis, ankylosing spondylitis bullous diseases, actinic keratoses, ulcerative colitis, Crohn's disease, alopecia areata, and diseases and disorders caused by the hepatitis C virus (HCV), the hepatitis B virus (HBV), or the human immunodeficiency virus (HIV).

66. The method of claim 60, wherein the infectious agent is a bacteria.

67. The method of claim 60, wherein the infectious agent is a virus.

68. The method of claim 60, wherein the infectious agent is a parasite.

69. The method of claim 66, wherein the bacteria is a Gram-negative or a Gram-positive bacteria.

70. The method of claim 69, wherein the Gram-negative bacteria is selected from the group consisting of Acinetobacter baumanii, Aggregatobacter actinomycetemcomitans, Bartonella bacilliformis, Bartonella, henselae, Bartonella quintana, Bifidobacterium Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trahomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida Pseudomonas fluorescens, Pseudomonas acidovorans, Rickettsiae, Salmonella enterica, Salmonella typhi, Salmonella paratyphi types A, B typhus, Salmonella, dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus and Yersinia pestitis.

71. The method of claim 69, wherein the Gram-positive bacteria selected from the group consisting of Actinomycetes, Bacillus anthracis, Bacillus subtilis, Clostridium tetani, Clostridium, perfingens, Clostridium botulinum, Clostridium tetani, Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, and Streptococcus mutants.

72. The method of claim 67, wherein the virus is selected from the group consisting of ebolavirus, hepatitis B virus, hepatitis C virus, herpes simplex virus, human immunodeficiency virus (HIV), human papillomavirus (HPV-6, HPV-11), human SARS coronavirus, influenza A virus, influenza B virus, influenza C virus, measles virus, rabies virus, poliovirus, SARS corona virus, and yellow fever virus.

73. The method of claim 68, wherein the parasite is selected from the group consisting of Acanthamoeba spp, American trypanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

74. The method of claim 56, further comprising administering to the subject one or more additional therapeutic agents or immune modulators, and combinations thereof.

75. The method of claim 74, wherein the one or more additional therapeutic agents is selected from an anti-microbial agent, such as an anti-bacterial agent, an anti-viral agent, or an anti-parasitic agent, an anti-cancer agent, or a therapeutic agent for the treatment of tuberculosis, meningitis, pneumonia, ulcer, sepsis, rhinitis, asthma, allergy, COPD, inflammatory bowel disease, arthritis, obesity, radiation-induced inflammation, psoriasis, atopic dermatitis, non-alcoholic steatohepatitis (NASH), Alzheimer's disease, systemic lupus, erythematosus (SLE), autoimmune thyroiditis (Grave's disease), multiple sclerosis, and ankylosing spondylitis bullous diseases.

76. The method of claim 74, wherein the one or more additional immune modulators is selected from the group consisting of an inhibitor or antagonist of an immune checkpoint regulator, a vaccine, preferably a vaccine against an immune checkpoint regulator, an immune stimulatory molecule, an agonist of an immune co-stimulatory molecule, a recombinant protein, and a T cell, preferably a chimeric antigen receptor T (CAR-T) cell.

77. The method of claim 76, wherein the immune checkpoint regulator is selected from the programed cell death 1 (PD-1) receptor (CD279), a ligand of PD-1 (e.g., PD-L1), cytotoxic T-lymphocyte associated protein 4 (CTLA4), tumor necrosis factor receptor superfamily member 9 (alternatively TNFRSF9, 4-1BB) and 4-1BB ligands, tumor necrosis factor receptor superfamily member 4 (alternatively TNFRSF4, OX40) and OX40 ligands, glucocorticoid-induced TNFR-related protein (GITR), Tumor Necrosis Factor Receptor Superfamily Member 7 (alternatively TNFRSF7, cluster of differentiation 27, CD27), TNFRSF25 and TNF-like ligand 1A (TL1A), TNF Receptor Superfamily Member 5 (alternatively TNFRSF5, CD40) and CD40 ligand, Herpesvirus entry mediator (HVEM)-tumor necrosis factor ligand superfamily member 14 (alternatively TNFSF14, LIGHT)-lymphotoxin alpha (LTA), herpesvirus entry mediator-(HVEM)-B- and T-lymphocyte attenuator (BTLA)-CD160 (alternatively TNFSF14), lymphocyte activating gene 3 (LAG3), T-cell immunoglobulin and mucin-domain containing-3 (TIM3), sialic-acid-binding immunoglobulin-like lectins (SIGLECs), inducible T-cell costimulator (ICOS) and ICOS ligand, B7-H3 (B7 family, alternatively CD276), V-set domain-containing T-cell activation inhibitor 1 (VTCN1, alternatively B7-H4), V-Type immunoglobulin domain-containing suppressor of T-cell activation (VISTA), human endogenous retrovirus-H long terminal repeat-associating protein 2 (HHLA2)-transmembrane and Immunoglobulin domain containing 2 (TMIGD2), butyrophilins, natural killer cell receptor 2B4 (alternatively NKR2B4, CD244) and B-Cell Membrane Protein (CD48), T-Cell Immunoreceptor with Immunoglobulin (Ig) and immunoreceptor tyrosine-based inhibition motif domains (TIGIT) and Poliovirus receptor (PVR) family members, killer-cell immunoglobulin-like receptors (KIRs), Immunoglobulin-like transcripts (ILTs) and leukocyte immunoglobulin-like receptor (LIRs), natural killer group protein 2 member D (NKG2D) and natural killer group protein 2 member A (NKG2A), major histocompatibility complex (MHC) class I polypeptide-related sequence A (MICA) and MHC class I polypeptide-related sequence B (MICB), natural killer cell receptor 2B4 (CD244), colony stimulating factor 1 receptor (CSF1R), indoleamine 2,3-dioxygenase (IDO), transforming growth factor beta (TGFβ), Adenosine-ecto-nucleotidase triphosphate diphosphohydrolase 1 (CD39)-5′-nucleotidase (CD73), C-X-C motif chemokine receptor 4 (CXCR4) and C-X-C motif chemokine ligand 12 (CXCL12), phosphatidylserine, signal regulatory protein alpha (SIRPA) and integrin associated protein (CD47), vascular endothelial growth factor (VEGF), and neuropilin.

78. The method of claim 76, wherein the one or more additional immune modulators is a vaccine.

79. The method of claim 76, in a method for treating cancer, wherein the vaccine is a vaccine against a tumor antigen.

80. The method of claim 79, wherein the tumor antigen is selected from glycoprotein 100 (gp100), mucin 1 (MUC1), and melanoma-associated antigen 3 (MAGEA3).

81. The method of claim 76, wherein the one or more additional immune modulators is a T cell, preferably a chimeric antigen receptor T cell.

82. The method of claim 76, wherein the one or more additional immune modulators is a recombinant protein, preferably selected from granulocyte-macrophage colony-stimulating factor (GM-CSF), interleukin 7 (IL-7), IL-12, IL-15, IL-18, and IL-21.

83. The method of claim 56, wherein the composition comprises a compound or a pharmaceutically acceptable salt of any one of claims 1 to 51.

84. The method of claim 76, wherein the one or more additional immune modulators is an inhibitor or antagonist of an immune checkpoint regulator, preferably a PD-1/PD-L1 inhibitor.

85. The method of claim 84, wherein the PD-1/PD-L1 inhibitor is selected from the group consisting of nivolumab, pembrolizumab, pidilizumab, BMS-936559, atezolizumab, durvalumab, and avelumab.

86. The method of any one of claim 57, wherein the cancer is selected from advanced melanoma, non-small cell lung cancer, renal cell carcinoma, bladder cancer, Hodgkin's lymphoma, liver cancer, gastric cancer, colon cancer, breast cancer, non-Hodgkin's lymphoma, prostate cancer, head and neck cancer, thyroid cancer, brain cancer, acute myeloid leukemia (AML), merkel cell carcinoma, multiple myeloma, cervical cancer, and sarcoma.

87. A method for treating cancer in a subject in need of such treatment, the method comprising administering to the subject a composition comprising a compound of claim 1, and an immune modulator selected from one or more of an inhibitor or antagonist of an immune checkpoint regulator, an immune stimulatory molecule, and an agonist of an immune co-stimulatory molecule.

88. The method of claim 87, wherein the inhibitor or antagonist of an immune checkpoint regulator is a PD-1/PD-L1 inhibitor.

89. The method of claim 88, wherein the PD-1/PD-L1 inhibitor is selected from the group consisting of nivolumab, pembrolizumab, pidilizumab, BMS-936559, atezolizumab, durvalumab, and avelumab.

90. The method of claim 87, wherein the immune modulator is selected from interferon alpha (INFα), a stimulator of interferon genes (“STING”) agonist, a TLR agonist (e.g., resquimod), and an anti-OX40 (CD134) agonist antibody.

91. The method of claim 87, wherein the immune modulator is an agonist of an immune co-stimulatory molecule.

92. The method of claim 91, wherein the agonist of an immune co-stimulatory molecule is an anti-OX40 (CD134) agonist antibody.

93. A method for treating a liver disease or disorder in a subject in need of such treatment, the method comprising administering a compound or a pharmaceutically acceptable salt of claim 1 to the subject.

94. The method of claim 93, wherein the liver disease or disorder is selected from liver cancer, non-alcoholic steatohepatitis (NASH), and a disease or disorder caused by infection with the hepatitis C virus (HCV) or the hepatitis B virus (HBV).

95. The method of claim 56, wherein the subject is a vertebrate.

96. The method of claim 56, wherein the subject is a human.

97. A vaccine composition or vaccine adjuvant composition comprising a compound or a pharmaceutically acceptable salt of claim 1.

98. A pharmaceutical composition, a vaccine composition, or a vaccine adjuvant composition comprising a compound or a pharmaceutically acceptable salt of claim 1.
